(19) United States
(12) Patent Application Publication
(10) Pub. No.: US 2022/0008519 A1
(43) Pub. Date: Jan. 13, 2022

(54) TREATMENT OF SEVERE ACUTE RESPIRATORY SYNDROME-RELATED CORONAVIRUS INFECTION WITH KLOTHO

(71) Applicant: Costa Rican Social Security Fund / Caja Costarricense de Seguro Social (CCSS), San José (CR)

(72) Inventor: Román Federico Macaya Hayes, Escazu (CR)

(21) Appl. No.: 17/073,685

(22) Filed: Oct. 19, 2020

Related U.S. Application Data
(60) Provisional application No. 63/050,008, filed on Jul. 9, 2020.

(51) Int. Cl. A61K 38/47; A61K 9/00; A61K 31/155; A61P 31/14

(52) U.S. Cl. CPC A61K 38/47; A61P 31/14; A61K 31/155; A61K 9/0019

(57) ABSTRACT

Methods and compositions for treating a severe acute respiratory syndrome-related coronavirus (SARS-CoV) infection in a subject in need are provided. In some aspects, a therapeutically effective amount of a Klotho polypeptide and/or a Klotho polynucleotide encoding a Klotho polypeptide is administered to the subject. In some other aspects, the subject is treated with a first therapy when the subject has diminished Klotho activity, and with a second therapy when the subject does not have diminished Klotho activity. Diminished Klotho activity is determined by comparing the amount of Klotho protein in a blood sample from the subject to a predetermined threshold. In particular, methods and compositions for treating SARS-CoV-2 infection are provided.

### CROSS REFERENCE TO RELATED APPLICATIONS

This application claims priority to U.S. Provisional Patent Application No. 63/050,008, filed Jul. 9, 2020, the content of which is hereby incorporated by reference, in its entirety, for all purposes.

### REFERENCE TO SEQUENCE LISTING

The Sequence Listing text copy submitted herewith via EFS-Web was created on Nov. 6, 2020, is entitled seglisting1276565001US_ST25.txt, is 31,691 bytes in size and is herein incorporated by reference in its entirety.

### BACKGROUND OF THE INVENTION

COVID-19 is characterized by diverse manifestations, ranging from asymptomatic infections that resolve without complications to severe cases and sudden death. Throughout the course of infection, the virus can present with any number of symptoms, including cough, fever, loss of smell, loss of taste, and shortness of breath, with the potential to develop into more extreme complications such as respiratory failure, hypoxemia, hypoxia, renal failure, multi-organ failure, micro-coagulation and thrombosis, stroke, gastrointestinal problems, and cytokine storm. While the mechanism of action of COVID-19 remains elusive, several risk factors have been identified, including hypertension, diabetes, obesity, smoking history, cancer, AIDS, asthma, and chronic obstructive pulmonary disease (COPD).

Amidst these diverse characteristics, one common factor is the well-documented correlation between COVID-19 susceptibility and age. For example, aging plays a role in contributing to the onset of risk factors for COVID-19. In addition, mortality from COVID-19 is higher in men than in women, in part because men age biologically faster than women. Another predictor of mortality from COVID-19 is the presence of age-related diseases. For example, a younger individual with age-related diseases such as diabetes and hypertension may be at higher risk for mortality than an older individual with no age-related diseases. In such cases, aging can be thought of as a hardwired biological process, culminating in cellular decay and/or functional decline that eventually develop into clinical complications. Accelerated or decelerated progression through the aging process, in some such instances, results in a biological age that either exceeds or falls short of the chronological age. Thus, the risk of developing age-related diseases, while statistically higher in chronologically older individuals, is ultimately linked to the underlying processes of biological aging. See, Blagosklonny, 2020, “From causes of aging to death from COVID-19,” Aging, 12 (11), 10004-10021.

Recent studies have focused on the use of anti-aging drugs, such as rapamycin, for the treatment of COVID-19. Rapamycin inhibits the mammalian/mechanistic target of rapamycin (mTOR) by binding to the mTORC1 subunit of the mTOR complex. See, Sargiacomo et al., 2020, “COVID-19 and chronological aging: senolytics and other anti-aging drugs for the treatment or prevention of coronavirus infection?” Aging 12(8). Nevertheless, these studies fail to identify the underlying mechanism for severe clinical complications. Alternative methods facilitating a more direct approach to diagnosis, monitoring and treatment can provide more efficient, targeted intervention of the clinical and health complications caused by novel coronavirus.

### BRIEF SUMMARY OF INVENTION

The present disclosure provides solutions to these and other problems by providing methods and compositions for the treatment of diseases caused by coronaviruses, including severe acute respiratory syndrome-related coronavirus 2 (SARS-CoV-2) infection, the agent known to cause COVID-19. For example, while no unifying agent or signaling pathway has been identified to date that can explain the diversity of clinical manifestations of this virus, the present disclosure provides methods and compositions comprising Klotho as a central agent to treat COVID-19 patients. Klotho is an anti-aging protein that has been shown to be involved in numerous biological processes that are consistent with the known mechanisms of SARS-CoV-2 infection and evolution of COVID-19 disease.

These findings place the Klotho signaling pathway at the center of a unified mechanism to explain the risk factors, symptoms, complications and evolution of COVID-19 disease, and suggest a direct or indirect down regulation of Klotho expression by SARS-CoV-2. This premise also suggests that Klotho-replacement therapy, as well as agents that upregulate Klotho expression, such as mTOR inhibitors, may find use for the treatment of COVID-19 patients, particularly those with risk factors. Finally, given that the medium and long-term health consequences of a SARS-CoV-2 infection are still unknown, public health programs should monitor recovered patients for the frequency of diseases that are linked to Klotho deficiency, especially given Klotho's role in Kawasaki disease in children, in age-correlated illnesses, such as Alzheimer's disease, and as a tumor suppressor. This disclosure provides methods for treating, or protecting against, the acute onset of clinical or health complications caused by acute respiratory syndrome-related coronavirus (SARS-CoV) infection in a subject in need thereof, as well as the medium and long-term clinical and health complications that can manifest themselves after a patient recovers from the acute complications from such an infection and tests negative for the presence of the virus.

Accordingly, in one aspect, the disclosure provides methods for treating, or protecting against, the acute, midterm or long-term onset of clinical or health complications caused by a severe acute respiratory syndrome-related coronavirus (SARS-CoV) infection in a subject in need thereof, by administering a therapeutically effective amount of a Klotho polypeptide to the subject. In some embodiments, the SARS-CoV infection is a severe acute respiratory syndrome-related coronavirus 2 (SARS-CoV-2) infection. In some embodiments, the Klotho polypeptide is an α-Klotho polypeptide, e.g., a human α-Klotho polypeptide. In some embodiments, the Klotho polypeptide is a β-Klotho polypeptide, e.g., a human β-Klotho polypeptide. In some embodiments, the Klotho polypeptide is a γ-Klotho polypeptide, e.g., a human γ-Klotho polypeptide.

In another aspect, the disclosure provides methods for treating, or protecting against, the acute, midterm or long-term onset of clinical or health complications caused by a severe acute respiratory syndrome-related coronavirus (SARS-CoV) infection in a subject in need thereof, by administering a Klotho polynucleotide encoding a Klotho polypeptide to the subject. In some embodiments, the SARS-CoV infection is a severe acute respiratory syndrome-related coronavirus 2 (SARS-CoV-2) infection. In some embodiments, the Klotho polypeptide is an α-Klotho polypeptide, e.g., a human α-Klotho polypeptide. In some embodiments, the Klotho polypeptide is a β-Klotho polypeptide, e.g., a human β-Klotho polypeptide. In some embodiments, the Klotho polypeptide is a γ-Klotho polypeptide, e.g., a human γ-Klotho polypeptide.

In another aspect, the disclosure provides methods for differentially treating, or protecting against, the acute, midterm or long-term onset of clinical or health complications caused by a severe acute respiratory syndrome-related coronavirus (SARS-CoV) infection in a subject in need thereof, based on the subject's Klotho protein levels and/or Klotho activity. In some embodiments, the methods include treating the subject with a first therapeutic regimen when the subject has diminished Klotho protein levels and/or Klotho activity, and with a second therapeutic regimen when the subject does not have diminished Klotho protein levels and/or Klotho activity. In some embodiments, the first therapeutic regimen includes administration of a Klotho polypeptide or a Klotho polynucleotide, as described herein. In some embodiments, the first therapeutic regimen includes more aggressive treatment than the second therapeutic regimen.

In another aspect, the present disclosure provides methods for treating, or protecting against, the acute, midterm or long-term onset of clinical or health complications caused by a coronavirus infection (e.g., SARS-CoV) in a subject in need thereof, by administering a treatment based on an underlying etiology of risk factors or complications associated with a severe coronavirus-mediated disease (e.g., SARS-CoV-2, SARS-CoV-1, and/or MERS). In some embodiments, the underlying risk factor is dyslipidemia and/or hyperlipidemia. In some embodiments, the underlying risk factor is inflammation. In some embodiments, the underlying risk factor is activation of the mTOR pathway. Accordingly, in one aspect, the present disclosure provides methods for treating a severe acute respiratory syndrome-related coronavirus (SARS-CoV) infection in a subject with hyperlipidemia and in need thereof, by administering a therapeutically effective amount of a lipid-reducing compound. In another aspect, the present disclosure provides methods for treating a severe acute respiratory syndrome-related coronavirus (SARS-CoV) infection in a subject in need thereof, by administering a therapeutically effective amount of an inhibitor of the NF-κB pathway. In another aspect, the present disclosure provides methods for treating a severe acute respiratory syndrome-related coronavirus (SARS-CoV) infection in a subject in need thereof, by administering a therapeutically effective amount of an inhibitor of the mTOR pathway.

### BRIEF DESCRIPTION OF DRAWINGS

FIG. 1A shows the amino acid sequence for isoform 1 of the human α-Klotho protein (SEQ ID NO:1).

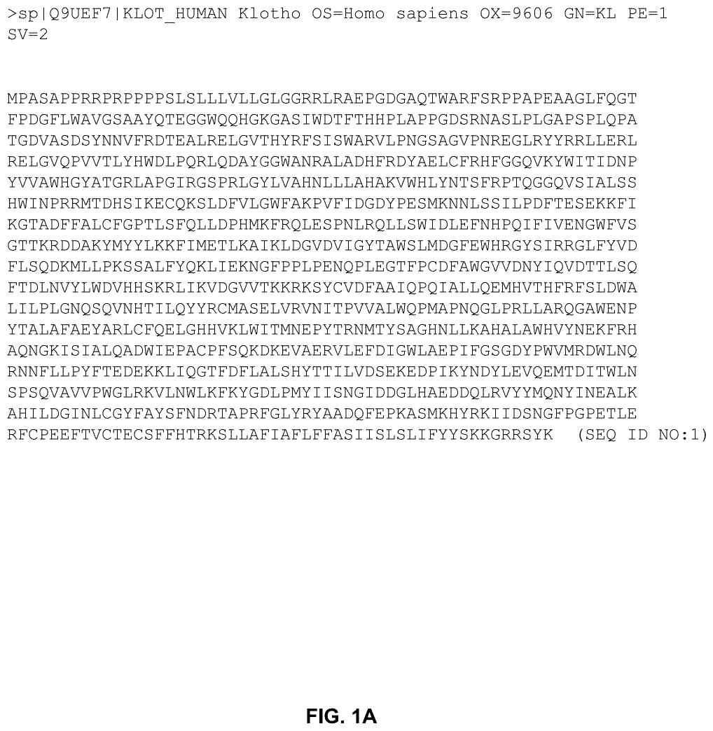

FIG. 1B shows the amino acid sequence for isoform 2 of the human α-Klotho protein (SEQ ID NO:4).

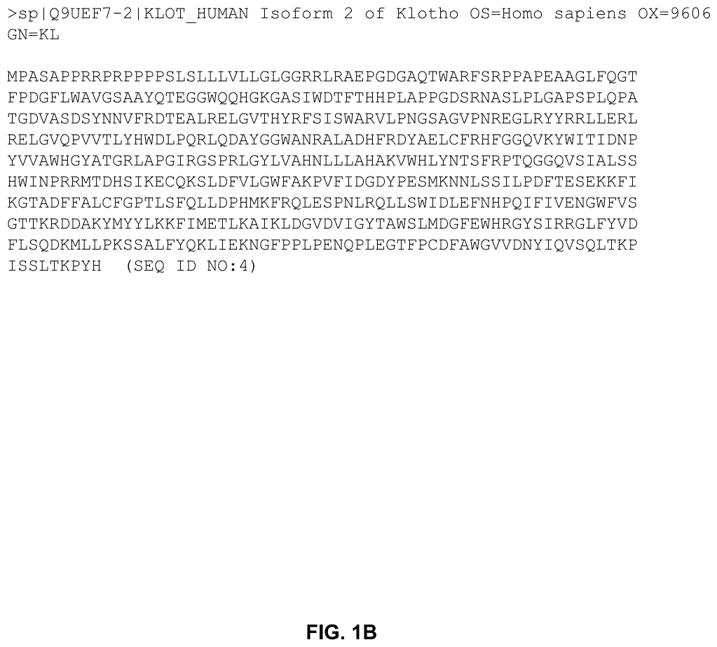

FIG. 2 shows the amino acid sequence for the human β-Klotho protein (SEQ ID NO:2).

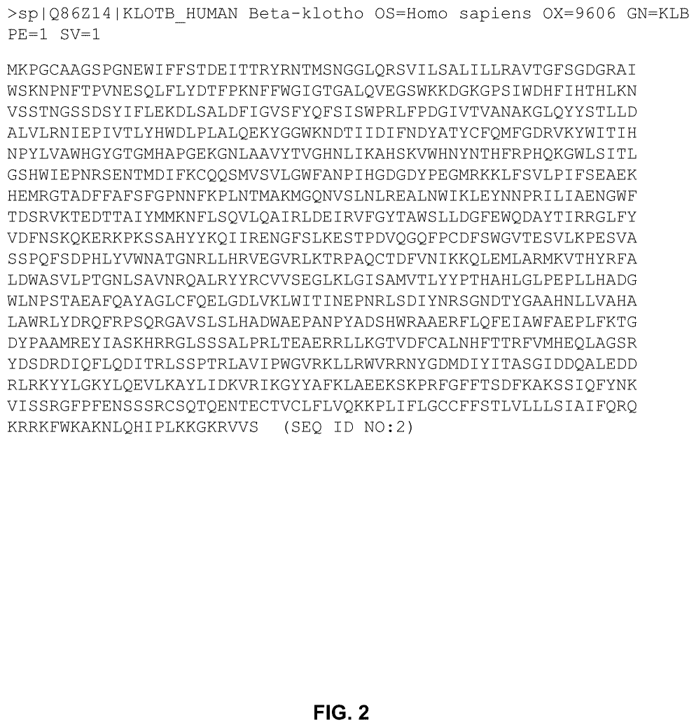

FIG. 3A shows the amino acid sequence for isoform 1 of the human γ-Klotho protein (SEQ ID NO:3).

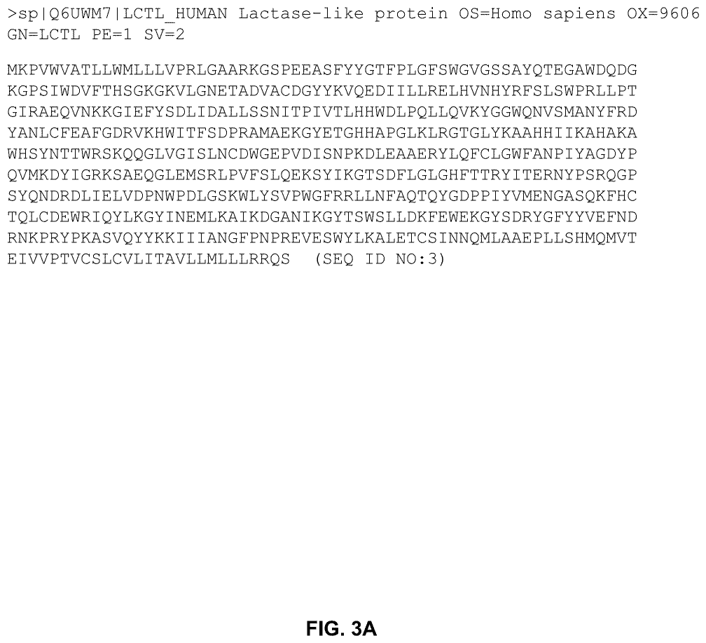

FIG. 3B shows the amino acid sequence for isoform 2 of the human γ-Klotho protein (SEQ ID NO:5).

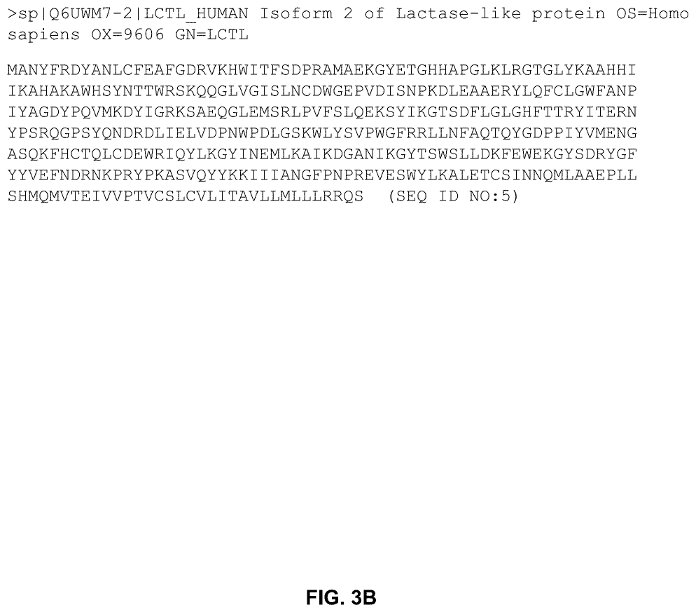

FIG. 4 illustrates a deleterious cascade generated by SARS-CoV-2-induced acute kidney injury, in accordance with some embodiments of the present disclosure. The figure illustrates the Klotho-FGF23 axis and the pathological mechanism of SARS-CoV-2 mediated depletion of ACE2 in the context of acute kidney injury (AKI). AKI exerts a pivotal role as it induces both an exponential increase in FGF23 levels and exponential decrease in Klotho, with adverse consequences such as ACE2 depletion, worsening of kidney function, inhibition of the canonical Klotho-FGF23 signaling and subsequent activation of off-target effects. ACE2 depletion induced by this coronavirus further aggravates not only the kidney injury but also acute respiratory distress syndrome.

### DETAILED DESCRIPTION OF INVENTION

### Introduction

As described above, there is a need in the art for improved methods of diagnosing, treating, monitoring, and preventing diseases caused by coronavirus infection, e.g., COVID-19, SARS, MERS, and the like. In particular, the occurrence of several coronavirus-mediated epidemics over the past twenty years, e.g., the SARS, MERS, and COVID-19 epidemics, underscores the need for better management of such diseases. The present disclosure provides such methods, based on the identification of the Klotho protein as a key mediator that protects against severe effects of such diseases.

Accordingly, in some aspects, methods are described for preventing or treating a coronavirus-mediated disease, e.g., COVID-19, SARS, MERS, and the like, by administering to a subject in need thereof a therapeutically effective amount of a Klotho polypeptide or a Klotho polynucleotide. Similarly, methods are described for providing a prognosis for the severity of a coronavirus-mediated disease, and/or monitoring the progression and/or treatment of such disease, by determining the level of a Klotho polypeptide and/or the level of a Klotho activity in a subject.

Similarly, in some aspects, methods are described for preventing or treating a coronavirus-mediated disease, e.g., COVID-19, SARS, MERS, and the like, by treating an underlying risk factor, associated with a severe form of the disease, that has been linked to Klotho function. For instance, as described further below, cytokine storms-known to downregulate Klotho expression—have been associated with severe COVID-19 disease. Accordingly, in some embodiments, methods for preventing or treating a coronavirus-mediated disease, e.g., COVID-19, include administration of an inhibitor of a cytokine or an inhibitor of a signaling pathway triggered by a cytokine that participates in a cytokine storm. In some embodiments, the inhibitor is an inhibitor of the NF-κB signaling pathway. Similarly, in some embodiments, the inhibitor is an inhibitor of the mTOR signaling pathway. As another non-limiting example, hyperlipidemia—also known to downregulate Klotho expression—has been associated with severe COVID-19 disease. Accordingly, in some embodiments, methods for preventing or treating a coronavirus-mediated disease, e.g., COVID-19, include administration of a lipid-lowering agent (e.g., a statin, bile acid binding resin, cholesterol absorption inhibitor, fibrate, niacin, or omega-3 fatty acid) particularly in subjects with hyperlipidemia. In some embodiments, the subject was not previously taking a lipid-lowering agent and/or was not previously diagnosed with hyperlipidemia.

SARS-CoV-2 is a novel coronavirus that has caused a global pandemic in which the total number of confirmed COVID-19 cases surpasses ten million, with a related death toll of over half a million. A surprising aspect of this coronavirus is the diversity of risk factors for complications, symptoms and health outcomes this virus can exhibit and cause in infected patients. Risk factors for complications include advanced age and health conditions that tend to be more prevalent in the elderly, such as hypertension, diabetes, obesity, COPD, cancer, chronic kidney disease, and smoking, among others. COVID-19 patients can show a wide array of symptoms, including loss of smell and taste, cough, fever, gastro-intestinal manifestations and fatigue. The evolution of a patient's experience with this disease can range from asymptomatic or mild symptoms to severe complications, including hypoxemia and hypoxia, acute respiratory distress syndrome (ARDS), renal failure, microcoagulation and thrombosis, Kawasaki disease in children, pulmonary embolism, stroke, multi-organ failure and cytokine release syndrome, requiring critical care, mechanical ventilation and possible death.

While the pace of advancement in the scientific understanding and knowledge of this virus and the evolution of COVID-19 disease has been remarkable, no unifying agent or signaling pathway has been identified to date that can explain the diversity of clinical manifestations of this virus. However, recent studies have highlighted the importance of aging and age-related diseases as risk factors for the onset and progression of severe clinical complications, including mortality, from COVID-19 infections.

Among other aspects, the present disclosure provides methods and compositions for diagnosing and treating coronavirus-mediated disease that are based on the discovery that Klotho may serve as a central agent in coronavirus-mediated disease, explaining the wide range of COVID-19 risk factors and clinical outcomes. Klotho is an anti-aging protein that has been shown to be involved in numerous biological processes that are consistent with the known mechanisms of SARS-CoV-2 infection and evolution of COVID-19 disease.

Early reports revealed that disruption of the gene that encodes the Klotho protein resulted in accelerated aging and decreased lifespan in mice, while overexpression of the gene extended lifespans by 30%. See, Kuro-o et al., 1997, “Mutation of the mouse Klotho gene leads to a syndrome resembling ageing,” Nature 390:45-51. The protein is highly evolutionarily conserved, and is found to be correlated with a number of age-related complications in humans. Decreased levels of serum Klotho aggravate aging-related processes and correlate strongly with the severe conditions COVID-19 can cause. For example, Klotho is downregulated in patients presenting known risk factors for severe clinical complications from COVID-19 disease, such as hypertension, diabetes, obesity, smoking history, chronic obstructive pulmonary disease (COPD), asthma, dyslipidemia and/or hyperlipidemia, and cancer, among other risk factors. For further examples detailing the role of Klotho in risk factors for COVID-19 complications, see Wolf et al., “Klotho as a tumor suppressor,” Oncogene 27 (2008); Zhou et al., “Klotho: a novel biomarker for cancer,” J Cancer Res Clin Oncol 141 (2015); Coelho et al., “Chronic nicotine exposure reduces klotho expression and triggers different renal and hemodynamic responses in klotho-haploinsufficient mice,” Am J Physiol Renal Physiol 314 (2018); Wang et al., “Klotho Gene Delivery Prevents the Progression of Spontaneous Hypertension and Renal Damage,” “Hypertension 54(4) (2009); Zhou et al., Klotho Depletion Contributes to Increased Inflammation in Kidney of the db db Mouse Model of Diabetes via RelA (Serine)⁵³⁶ Phosphorylation,” Diabetes 60(7) (2011); Sang et al., “Decreased plasma α-Klotho predict progression of nephropathy with type 2 diabetic patients,” J Diab Comp 30(5) (2016); Amitani et al., “Plasma klotho levels decrease in both anorexia nervosa and obesity,” Nutrition 29(9) (2013); Giannubilo et al., “Placental klotho protein in preeclampsia: A possible link to long term outcomes,” Preg Hypertens 2(3) (2012); Milovanov et al., “Impact of Anemia Correction on the Production of the Circulating Morphogenetic Protein α-Klotho in Patients With Stages 3B-4 Chronic Kidney Disease: A New Direction of Cardionephroprotection,” Ter Arkh 88(6) (2016); Hariyanto and Kurniawan, “Dyslipidemia is associated with severe coronavirus disease 2019 (COVID-19) infection,” Diabetes Metab Syndr 14(5) (2020); and Sastre et al., “Hyperlipidemia-Associated Renal Damage Decreases Klotho Expression in Kidneys from ApoE Knockout Mice,” PLoS One 8(12) (2013), each of which is hereby incorporated by reference herein in its entirety. Notably, higher α-Klotho levels have been observed in women compared to lower α-Klotho levels in men, which correlates with the higher mortality from COVID-19 observed in men. See, Behringer et al., “Aging and sex affect soluble alpha klotho levels in bonobos and chimpanzees,” Front Zool 15(35) (2018), which is hereby incorporated by reference herein in its entirety.

Klotho downregulation is also correlated with high phosphate levels in the bloodstream, respiratory failure, anosmia, hypoxia and hypoxemia, kidney failure, diabetic shock, hypertension, abnormal blood ferritin levels, Kawasaki disease in children, coagulation abnormalities, ischemic stroke, gastrointestinal abnormalities, multi-organ failure, and cytokine storm. These have been identified as complications related to both aging and severe COVID-19 infections.

For example, increased Klotho levels have a nephron-protective role, whereas decreased Klotho levels are associated with acute and chronic kidney diseases. See, Vahed et al., “Klotho and Renal Fibrosis,” Nephrourol Mon 5(5) (2013); Hu et al., “Klotho and kidney disease,” J Nephrol 23(Suppl 16) (2010); and Milovanova et al., “Significance of the Morphogenetic Proteins FGF-23 and Klotho as Predictors of Prognosis of Chronic Kidney Disease,” Ter Arkh 86(4) (2014), each of which is hereby incorporated by reference herein in its entirety. Klotho deficiency was also linked to abnormalities observed in COVID-19 complications including atherosclerosis, hyperphosphatemia, emphysema, chronic obstructive pulmonary disease, hypertension, and stroke caused by cardioembolism. See, Levi et al., “Coagulation abnormalities and thrombosis in patients with COVID-19,” Lancet Haematol 7(6) (2020); Talotta et al., “Measurement of Serum Alpha-Klotho in Systemic Sclerosis Patients: Results from A Pivotal Study,” Annals Rheum Dis 75(Suppl 2) (2016); Gao et al., “Klotho expression is reduced in COPD airway epithelial cells: effects on inflammation and oxidant injury,” Clin Sci Lond 129(12) (2015); Xie et al., “COVID-19 Complicated by Acute Pulmonary Embolism,” Radiology Card Im 2(2) (2020); Pako et al., “Decreased Levels of Anti-Aging Klotho in Obstructive Sleep Apnea,” Rejuv Res 23(3) (2019); Kim et al., “Klotho Is a Genetic Risk Factor for Ischemic Stroke Caused by Cardioembolism in Korean Females,” Neurosci Lett 407(3) (2006); and Martin-Nunez et al., “Association between serum levels of Klotho and inflammatory cytokines in cardiovascular disease: a case-control study,” Aging 12(2) (2020), each of which is hereby incorporated by reference herein in its entirety. Conversely, overexpression of Klotho was reported to significantly decrease neuroinflammatory mechanisms, thus exerting a protective effect against ischemic brain injury. See, Zhou et al., “Protective Effect of Klotho against Ischemic Brain Injury Is Associated with Inhibition of RIG-I/NF-κB Signaling,” Front Pharmacol 8 (2017), which is hereby incorporated by reference herein in its entirety.

The overproduction of proinflammatory cytokines that can result in multiorgan injury in COVID-19 is also linked to low Klotho expression, as evidenced by the downregulation of Klotho by inflammatory mediators TWEAK and TNF-α as well as the inhibition of IL-6 by Klotho itself. Similarly, low Klotho expression has been reported to exacerbate sepsis and multiple organ dysfunction. See, Jose et al., “COVID-19 cytokine storm: the interplay between inflammation and coagulation,” The Lancet Resp Med 8(6) (2020); Moreno et al., “The Inflammatory Cytokines TWEAK and TNFα Reduce Renal Klotho Expression through NFκB,” JASN 22(7) (2011); Xia et al., “Klotho Contributes to Pravastatin Effect on Suppressing IL-6 Production in Endothelial Cells,” Mediators Inflam 2193210 (2016); and Jorge et al., “Klotho Deficiency Aggravates Sepsis-Related Multiple Organ Dysfunction,” Am J Physiol Renal Physiol 316(3) (2019), each of which is hereby incorporated by reference herein in its entirety.

In addition, downregulation of Klotho has been associated with anorexia, shedding light on a new possible risk factor for severe COVID-19 complications. The role of the Klotho signaling pathway in the evolution of kidney failure, Alzheimer's disease and certain cancers raises the prospect of important health complications that may be attributable to COVID-19 as mid-term to long-term consequences of SARS-CoV-2 infection. For example, levels of Klotho are inversely correlated with onset of Alzheimer's disease, senility, and dementia, and these cognitive impairments are correlated with chronic kidney disease, which have also been described above as being linked to decreased Klotho expression and aging. See, Dubal et al., “Life extension factor klotho enhances cognition,” Cell Rep 7(4) (2014); and Zeng et al., “Lentiviral vector-mediated overexpression of Klotho in the brain improves Alzheimer's disease-like pathology and cognitive deficits in mice,” Neurobiol Ag 78 (2019), each of which is hereby incorporated by reference herein in its entirety. The loss of smell and taste, one of the symptoms of COVID-19 infection, can also occur during the aging process. See also, Boyce and Shone, “Effects of ageing on smell and taste,” Postgrad Med J. 82(966) (2006), which is hereby incorporated by reference herein in its entirety.

Interestingly, the higher levels of serum Klotho found in children versus adults appears to explain the low susceptibility of children to severe COVID-19 complications, with the exception of children with Kawasaki disease, who exhibit lower Klotho expression levels. See, Falcini et al, “Circulating levels of Klotho in Kawasaki disease: A possible new marker of vascular damage?” Abstract, ACR/ARHP Sci Meet (2011), which is hereby incorporated by reference herein in its entirety. A deeper exploration of these observations reveals a potential risk factor and/or complication for COVID-19 in the onset of puberty, as highlighted by the associations found between Kallman syndrome, Klotho expression, and anosmia. Kallman syndrome is a genetic disorder characterized by the delayed onset or absence of puberty and is frequently accompanied by a loss of smell. Hypogonadotropic hypogonadism, another symptom characteristic of Kallman syndrome, is thought to be mediated by fibroblast growth factor receptor 1 (FGFR1) through the FGFR1/FGF21/KLB signaling pathway, where β-Klotho serves as the obligate co-receptor for the metabolic regulator FGF21 in conjunction with FGFR1. In addition to the onset of puberty and anosmia, the FGFR1/FGF21/KLB signaling pathway is also implicated in the response to starvation and other metabolic stresses, and β-Klotho mutations are further linked to decreased fertility and metabolic disorders including obesity and insulin resistance. See, for example, Misrahi, “β-Klotho sustains postnatal GnRH biology and spins the thread of puberty,” EMBO Mol Med 9(10) (2017); Cho et al., “Nasal Placode Development, GnRH Neuronal Migration and Kallmann Syndrome,” Front Cell Dev Biol 7(121) (2019); Goetz et al., “Klotho Coreceptors Inhibit Signaling by Paracrine Fibroblast Growth Factor 8 Subfamily Ligands,” Mol Cell Biol 32(10) (2012); and Xu et al., “KLB, encoding b-Klotho, is mutated in patients with congenital hypogonadotropic hypogonadism,” EMBO Mol Med 9(10) (2017), each of which is hereby incorporated by reference herein in its entirety.

Putative adjuvant therapies for COVID-19, such as iron chelators, zinc and vitamin D, are also associated with upregulated levels of Klotho. See, Vargas-Vargas and Cortes-Rojo, “Ferritin levels and COVID-19,” Rev Panam Salud Publica 44 (2020); Skalny et al., “Zinc and respiratory tract infections: Perspectives for COVID-19,” Int J Mol Med 46(1) (2020); Azimzadeh et al., “Effect of vitamin D supplementation on klotho protein, antioxidant status and nitric oxide in the elderly: A randomized, double-blinded, placebo-controlled clinical trial,” Euro J Int Med 35 (2020); Torres et al., “Klotho: An antiaging protein involved in mineral and vitamin D metabolism,” Kidney Int 71 (2007); and Shardell et al., “Serum 25-Hydroxyvitamin D, Plasma Klotho, and Lower-Extremity Physical Performance Among Older Adults: Findings From the InCHIANTI Study,” J Gerontol A Bio Sci Med Sci 70(9) (2015), each of which is hereby incorporated by reference herein in its entirety. Additional examples of factors that regulate or correlate with Klotho expression and/or Klotho levels are detailed in Table 1.

TABLE 1
Factors regulating the expression of Klotho.

| Factor | Reference |
|---|---|
| Decrease | |
| Kidney | |
| Aging | Nabeshima et al. |
| High-phosphate diet | Morishita et al. |
| Lipopolysaccharides | Ohyama et al. |
| Chronic renal failure in human | Koh et al. |
| Estrogens therapy | Oz et al. (abstract ASBMR 2006) |
| Hydrogen peroxide (oxidant stress) | Mitobe et al. |
| Ischemia-reperfusion injury model | Sugiura et al. |
| Spontaneously hypertensive rat | Aizawa et al. and Nagai et al. |
| Rat with 5/6 nephrectomy | Aizawa and Vonend et al. |
| Deoxycorticosterone acetate-salt hypertensive rat | Aizawa et al. |
| Noninsulin-dependent diabetes mellitus rat (the | Aizawa et al. |
| Otsuka Long-Evans Tokushima Fatty rat) | |
| Acute myocardial infarction | Aizawa et al. |
| Renal cell carcinoma | Yahata et al. |
| Angiotensin II | Ishizaka et al. |
| Iron-dextran | Saito et al. |
| Mevalonate, GGPP, and FPP | Narumiya et al. |
| Heart | |
| Aging | Nabeshima et al. |
| Liver | |
| Aging | Shih et al. |
| Lung | |
| Aging | Nabeshima et al. |
| Increase | |
| Kidney | |
| Low-phosphate diet | Morishita, Takaiwa (abstract ASBMR 2006) |
| High Ca + PO4 | Yu et al. (abstract ASBMR 2006) |
| Zinc orotate | Morishita et al. |
| 1,25(OH)₂D₃ | Tsujikawa et al. |
| Statins (atorvastatin and pravastatin) | Narumiya et al. |
| Rho-kinase inhibitor (Y27632) | Narumiya et al. |
| Adipocytes | |
| Triiodothyroxline | Mizuno et al. |
| PPARλ agonist | Chihara et al. |

See, for example, M. Kuro-o et al., “Mutation of the mouse klotho gene leads to a syndrome resembling ageing,” Nature, 390 (1997), pp. 45-51; H. Kurosu et al., “Suppression of aging in mice by the hormone klotho,” Science, 309 (2005), pp. 1829-1833; Q. Chang et al., “The beta-glucuronidase klotho hydrolyzes and activates the TRPV5 channel,” Science, 310 (2005), pp. 490-493; H. Kurosu et al., “Regulation of fibroblast growth factor-23 signaling by klotho,” J Biol Chem, 281 (2006), pp. 6120-6123; Y. Matsumura et al., “Identification of the human klotho gene and its two transcripts encoding membrane and secreted klotho protein,” Biochem Biophys Res Commun, 242 (1998), pp. 626-630; O. Tohyama et al., “Klotho is a novel beta-glucuronidase capable of hydrolyzing steroid beta-glucuronides,” J Biol Chem, 279 (2004), pp. 9777-9784; I. S. Mian, “Sequence, structural, functional, and phylogenetic analyses of three glycosidase families,” Blood Cells Mol Dis, 24 (1998), pp. 83-100; S. A. Li et al., “Immunohistochemical localization of klotho protein in brain kidney and reproductive organs of mice,” Cell Struct Funct, 29 (2004), pp. 91-99; M. Kamemori et al., “Expression of klotho protein in the inner ear,” Hear Res, 171 (2002), pp. 103-110; K. Takeshita et al., “Sinoatrial node dysfunction and early unexpected death of mice with a defect of klotho gene expression,” Circulation, 109 (2004), pp. 1776-1782; M. Imai et al., “Klotho protein activates the PKC pathway in the kidney and testis and suppresses 25-hydroxyvitamin D3 lalpha-hydroxylase gene expression,” Endocrine, 25 (2004), pp. 229-234; M. Yamamoto et al., “Regulation of oxidative stress by the anti-aging hormone klotho,” J Biol Chem, 280 (2005), pp. 38029-38034; I. Urakawa et al., “Klotho converts canonical FGF receptor into a specific receptor for FGF23,” Nature, 444 (2006), pp. 770-774; N. Koh et al., “Severely reduced production of klotho in human chronic renal failure kidney,” Biochem Biophys Res Commun, 280 (2001), pp. 1015-1020; O. Vonend et al., “Modulation of gene expression by moxonidine in rats with chronic renal failure,” Nephrol Dial Transplant, 19 (2004), pp. 2217-2222; H. Sugiura et al., “Klotho reduces apoptosis in experimental ischaemic acute renal failure,” Nephrol Dial Transplant, 20 (2005), pp. 2636-2645; H. Aizawa et al., “Downregulation of the Klotho gene in the kidney under sustained circulatory stress in rats,” Biochem Biophys Res Commun, 249 (1998), pp. 865-871; R. Nagai et al., “Endothelial dysfunction in the klotho mouse and downregulation of klotho gene expression in various animal models of vascular and metabolic diseases,” Cell Mol Life Sci, 57 (2000), pp. 738-746; K. Yahata et al., “Molecular cloning and expression of a novel klotho-related protein,” J Mol Med, 78 (2000), pp. 389-394; N. Ishizaka et al., “Angiotensin II regulates klotho gene expression,” Nippon Rinsho, 60 (2002), pp. 1935-1939; H. Mitani et al., “In vivo klotho gene transfer ameliorates angiotensin II-induced renal damage,” Hypertension, 39 (2002), pp. 838-843; M. Mitobe et al., “Oxidative stress decreases klotho expression in a mouse kidney cell line,” Nephron Exp Nephrol, 101 (2005), pp. e67-e74; Y. Nabeshima, “Ectopic calcification in Klotho mice,” Clin Calcium, 12 (2002), pp. 1114-1117; P. H. Shih and G. C. Yen, “Differential expressions of antioxidant status in aging rats: the role of transcriptional factor Nrf2 and MAPK signaling pathway,” Biogerontology (2006) (July 19, online); Y. Chihara et al., “Klotho protein promotes adipocyte differentiation,” Endocrinology, 147 (2006), pp. 3835-3842; A. Bektas et al., “Klotho gene variation and expression in 20 inbred mouse strains,” Mamm Genome, 15 (2004), pp. 759-767; L. Kappeler et al., “Ageing, genetics and the somatotropic axis,” Med Sci (Paris), 22 (2006), pp. 259-265; M. Ikushima et al., “Anti-apoptotic and anti-senescence effects of klotho on vascular endothelial cells,” Biochem Biophys Res Commun, 339 (2006), pp. 827-832; Y. Saito et al., “In vivo klotho gene delivery protects againstendothelial dysfunction in multiple risk factor syndrome,” Biochem Biophys Res Commun, 276 (2000), pp. 767-772; Y. Saito et al., “Klotho protein protects against endothelial dysfunction,” Biochem Biophys Res Commun, 248 (1998), pp. 324-329; R. H. Unger, “Klotho-induced insulin resistance: a blessing in disguise?” Nat Med, 12 (2006), pp. 56-57; D. E. Arking et al., “Association of human aging with a functional variant of klotho,” Proc Natl Acad Sci USA, 99 (2002), pp. 856-861; N. M. Xiao et al., “Klotho is a serum factor related to human aging,” Chin Med J (Engl), 117 (2004), pp. 742-747; H. Kawaguchi et al., “Independent impairment of osteoblast and osteoclast differentiation in klotho mouse exhibiting low-turnover osteopenia,” J Clin Invest, 104 (1999), pp. 229-237; H. Kawaguchi et al., “Cellular and molecular mechanism of low-turnover osteopenia in the klotho-deficient mouse,” Cell Mol Life Sci, 57 (2000), pp. 731-737; K. Kawano et al., “Klotho gene polymorphisms associated with bone density of aged postmenopausal women,” J Bone Miner Res, 17 (2002), pp. 1744-1751; N. Ogata et al., “Association of klotho gene polymorphism with bone density and spondylosis of the lumbar spine in postmenopausal women,” Bone, 31 (2002), pp. 37-42; J. A. Riancho et al., “Association of the F352V variant of the Klotho gene with bone mineral density,” Biogerontology (2006) (July 19, online); K. Morishita et al., “The progression of aging in klotho mutant mice can be modified by dietary phosphorus and zinc,” J Nutr, 131 (2001), pp. 3182-3188; H. Tsujikawa et al., “Klotho, a gene related to a syndrome resembling human premature aging, functions in a negative regulatory circuit of vitamin D endocrine system,” Mol Endocrinol, 17 (2003), pp. 2393-2403; M. S. Razzaque and B. Lanske, “Hypervitaminosis D and premature aging: lessons learned from Fgf23 and Klotho mutant mice,” Trends Mol Med, 12 (2006), pp. 298-305; M. S. Razzaque et al., “Premature aging-like phenotype in fibroblast growth factor 23 null mice is a vitamin D-mediated process,” FASEB J, 20 (2006), pp. 720-722; S. Tsuruoka et al., “Defect in parathyroid-hormone-induced luminal calcium absorption in connecting tubules of Klotho mice,” Nephrol Dial Transplant, 21 (2006), pp. 2762-2767; B. C. van der Eerden et al., “The epithelial Ca2+ channel TRPV5 is essential for proper osteoclastic bone resorption,” Proc Natl Acad Sci USA, 102 (2005), pp. 17507-17512; H. Segawa et al., “Correlation between hyperphosphatemia and type II Na/Pi cotransporter activity in klotho mice,” Am J Physiol Renal Physiol, 292 (2006), pp. F769-F779; K. Yahata et al., “Regulation of stanniocalcin 1 and 2 expression in the kidney by klotho gene,” Biochem Biophys Res Commun, 310 (2003), pp. 128-134; K. Saito et al., “Iron chelation and a free radical scavenger suppress angiotensin II-induced downregulation of klotho, an anti-aging gene, in rat,” FEBS Lett, 551 (2003), pp. 58-62; Y. Ohyama et al., “Molecular cloning of rat klotho cDNA: markedly decreased expression of klotho by acute inflammatory stress,” Biochem Biophys Res Commun, 251 (1998), pp. 920-925; H. Narumiya et al., “HMG-CoA reductase inhibitors up-regulate anti-aging klotho mRNA via RhoA inactivation in IMCD3 cells,” Cardiovasc Res, 64 (2004), pp. 331-336; and I. Mizuno et al., “Upregulation of the klotho gene expression by thyroid hormone and during adipose differentiation in 3T3-L1 adipocytes,” Life Sci, 68 (2001), pp. 2917-2923, each of which is hereby incorporated herein by reference in its entirety.

The above findings are consistent with the placement of the Klotho signaling pathway at the center of a unified mechanism to explain the risk factors, symptoms, complications and evolution of COVID-19 disease, and suggest a direct or indirect down regulation of Klotho expression by SARS-CoV-2. This premise also suggests that Klotho-replacement therapy, as well as agents that upregulate Klotho expression, such as mTOR inhibitors, may find use for the treatment of the acute manifestations of COVID-19 in patients, particularly those with risk factors. Finally, given that the medium and long-term health consequences of a SARS-CoV-2 infection are still unknown, public health programs should monitor recovered patients for the frequency of diseases that are linked to Klotho deficiency, especially given Klotho's role in age-correlated illnesses such as Alzheimer's disease, and as a tumor suppressor.

The Klotho protein is involved in the mTOR pathway and functions as a target of mTOR inhibition. Agents that inhibit mTOR, such as such as rapamycin, also known as sirolimus, rapamycin analogues, everolimus, metformin, senolytics, conventional and investigational NAD+ boosters, and/or other inhibitors of the mTOR pathway, may play a role in delaying aging by indirectly upregulating and/or blocking inhibition of Klotho. These compounds may also proove their therapeutic value in the treatment of acute, as well as mid-term and long-term COVID-19 complications. As provided herein, treatment and/or prevention of COVID-19 risk factors and/or complications include, in some embodiments, inhibitors of any of the mediators and intermediates of the mTOR pathway. See, for example, Cavanagh et al., “Angiotensin II blockade: how its molecular targets may signal to mitochondria and slow aging. Coincidences with calorie restriction and mTOR inhibition,” Am J Physiol Heart Circ Physiol 309 (2015); Zhavoronkov, “Geroprotective and senoremediative strategies to reduce the comorbidity, infection rates, severity, and lethality in gerophilic and gerolavic infections,” Aging 12(8) (2020); Sargiacomo et al., “COVID-19 and chronological aging: senolytics and other anti-aging drugs for the treatment or prevention of coronavirus infection?” Aging 12(8) (2020); Zhou et al., “Network-based drug repurposing for novel coronavirus 2019-nCoV/SARS-CoV-2,” Cell Dis 6(14) (2020); Maiese, “The Mechanistic Target of Rapamycin (mTOR): Novel Considerations as an Antiviral Treatment,” Curr Neurovas Res 17 (2020); and Wang et al., “Adjuvant Treatment With a Mammalian Target of Rapamycin Inhibitor, Sirolimus, and Steroids Improves Outcomes in Patients With Severe H1N1 Pneumonia and Acute Respiratory Failure,” Crit Care Med 42(2) (2014), each of which is hereby incorporated by reference herein in its entirety.

In some embodiments, inhibitors of any of the mediators of the risk factors and/or complications associated with COVID-19 detailed above. For example, inhibition of the NF-κB pathway can ameliorate the inflammatory processes leading to cytokine storm and/or multi-organ failure, reducing the severity and/or preventing the progression of COVID-19 infection. In some embodiments, low-density lipoprotein (LDL)-reducing treatments, such as statins, fibrates, and/or PCSK9 inhibitors, can also prevent the occurrence of COVID-19 risk factors such as dyslipidemia and/or hyperlipidemia. In some embodiments, two or more treatments are combined for an additive and/or synergistic effect. For example, activation of the NF-κB pathway has been shown to play a role in hyperlipidemia and oxidative LDL-mediated downregulation of Klotho. In some such embodiments, a therapeutic composition comprises an inhibitor of the NF-κB pathway and a LDL-reducing agent. See, Sastre et al., “Hyperlipidemia-Associated Renal Damage Decreases Klotho Expression in Kidneys from ApoE Knockout Mice,” PLoS One 8(12) (2013), which is hereby incorporated by reference herein in its entirety.

Based on its central role in COVID-19-associated complications, Klotho provides an attractive candidate for targeted therapy and other clinical and epidemiological procedures. Accordingly, the present disclosure utilizes Klotho as a candidate target for therapeutic intervention due to its role in aging and in age-related risk factors and diseases associated with COVID-19. In some embodiments, therapeutic interventions include prophylaxis (e.g., treatments for the prevention of COVID-19 infection), treatments for the amelioration of COVID-19 risk factors (e.g., underlying conditions), treatments for the amelioration of COVID-19 complications (e.g., symptoms), and/or any combinations thereof. In some embodiments, any of the therapeutic interventions include, but are not limited to, anti-viral treatments. In some embodiments, any of said therapeutic interventions are targeted towards pathways and/or processes mediated by Klotho. Additionally, in some embodiments, therapeutic interventions include treatments that improve downstream health after eradication of viral infection, including but not limited to longitudinal or multi-stage treatment regimens.

In one aspect, the disclosure provides methods for treating the clinical complications of a severe acute respiratory syndrome-related coronavirus (SARS-CoV) infection, as well as the possible mid-term and long-term health consequences of COVID-19 disease, in a subject in need thereof, by administering a therapeutically effective amount of a Klotho polypeptide to the subject. In some embodiments, the infection (e.g., SARS-CoV infection) is a severe acute respiratory syndrome-related coronavirus 2 (SARS-CoV-2) infection, the agent known to cause COVID-19. In some embodiments, the Klotho polypeptide is an α-Klotho polypeptide, e.g., a human α-Klotho polypeptide. In some embodiments, the Klotho polypeptide is a β-Klotho polypeptide, e.g., a human β-Klotho polypeptide. In some embodiments, the Klotho polypeptide is a γ-Klotho polypeptide, e.g., a human γ-Klotho polypeptide.

In another aspect, the disclosure provides methods for treating the clinical complications of a severe acute respiratory syndrome-related coronavirus (SARS-CoV) infection, as well as the possible mid-term and long-term health consequences of COVID-19 disease, in a subject in need thereof, by administering a Klotho polynucleotide encoding a Klotho polypeptide to the subject. In some embodiments, the SARS-CoV infection is a severe acute respiratory syndrome-related coronavirus 2 (SARS-CoV-2) infection, the agent known to cause COVID-19. In some embodiments, the Klotho polypeptide is an α-Klotho polypeptide, e.g., a human α-Klotho polypeptide. In some embodiments, the Klotho polypeptide is a β-Klotho polypeptide, e.g., a human β-Klotho polypeptide. In some embodiments, the Klotho polypeptide is a γ-Klotho polypeptide, e.g., a human γ-Klotho polypeptide.

In another aspect, the disclosure provides methods for differentially treating the clinical complications of a severe acute respiratory syndrome-related coronavirus (SARS-CoV) infection, as well as the possible mid-term and long-term health consequences of COVID-19 disease, in a subject in need thereof, based on the subject's Klotho protein levels and/or Klotho activity. In some embodiments, the methods include treating the subject with a first therapeutic regimen when the subject has diminished Klotho protein levels and/or Klotho activity, and with a second therapeutic regimen when the subject does not have diminished Klotho protein levels and/or Klotho activity. In some embodiments, the first therapeutic regimen includes administration of a Klotho polypeptide or a Klotho polynucleotide, as described herein. In some embodiments, the first therapeutic regimen includes more aggressive treatment than the second therapeutic regimen.

In some embodiments, the methods and compositions provided herein are useful for the treatment of human coronavirus-related diseases. For example, in some embodiments, the SARS-CoV infection is a severe acute respiratory syndrome-related coronavirus (SARS-CoV-1) infection, the agent known to cause SARS. In some embodiments, the methods and compositions provided herein are useful for the treatment of Middle East respiratory syndrome-related coronavirus (MERS-CoV), the agent known to cause MERS. In some embodiments, the Klotho polypeptide is an α-Klotho polypeptide, e.g., a human α-Klotho polypeptide. In some embodiments, the Klotho polypeptide is a β-Klotho polypeptide, e.g., a human β-Klotho polypeptide. In some embodiments, the Klotho polypeptide is a γ-Klotho polypeptide, e.g., a human γ-Klotho polypeptide.

### Definitions

As used herein, the term “administration” refers to a process of delivering a treatment (e.g., a therapeutic agent and/or a therapeutic composition) to a subject. An administration may be performed using oral, intravenous, intraocular, subcutaneous, and/or intramuscular means. An administration may be systemic or directed, in which the treatment is preferentially delivered to a first location in a subject as compared a second location or systemic distribution of the agent. For example, in one embodiment, directed administration of a therapeutic agent results in at least a two-fold increase in the ratio of therapeutic agent delivered to a targeted site to therapeutic agent delivered to a non-targeted site, as compared to the ratio following systemic or non-directed administration. In other embodiments, directed administration of a therapeutic agent results in at least a 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 200-fold, 300-fold, 400-fold, 500-fold, 750-fold, 1000-fold, or greater increase in the ratio of therapeutic agent delivered to a targeted site to therapeutic agent delivered to a non-targeted site, as compared to the ratio following systemic or non-directed administration.

As used herein, the term “amino acid” refers to naturally occurring and non-natural amino acids, including amino acid analogs and amino acid mimetics that function in a manner similar to the naturally occurring amino acids. Naturally occurring amino acids include those encoded by the genetic code, as well as those amino acids that are later modified, e.g., hydroxyproline, γ-carboxyglutamate, and O-phosphoserine. Naturally occurring amino acids can include, e.g., D- and L-amino acids. The amino acids used herein can also include non-natural amino acids. Amino acid analogs refer to compounds that have the same basic chemical structure as a naturally occurring amino acid, e.g., any carbon that is bound to a hydrogen, a carboxyl group, an amino group, and an R group, e.g., homoserine, norleucine, methionine sulfoxide, or methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The nucleotide sequences that encode one or more Klotho polypeptides herein may be identical to the coding sequence provided herein or may be a different coding sequence, which sequence, as a result of the redundancy or degeneracy of the genetic code, encodes the same polypeptides as the coding sequences provided herein. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each variation of a nucleic acid which encodes a same polypeptide is implicit in each described sequence with respect to the expression product, but not with respect to actual gene therapy constructs.

As to amino acid sequences, one of ordinary skill in the art will recognize that individual substitutions, deletions or additions to a nucleic acid or peptide sequence that alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

Conservative amino acid substitutions providing functionally similar amino acids are well known in the art. Dependent on the functionality of the particular amino acid, e.g., catalytic, structural, or sterically important amino acids, different groupings ofamino acid may be considered conservative substitutions for each other. Table 2 provides groupings of amino acids that are considered conservative substitutions based on the charge and polarity of the amino acid, the hydrophobicity ofthe amino acid, the surface exposure/structural nature ofthe amino acid, and the secondary structure propensity of the amino acid.

TABLE 2
Groupings of conservative amino acid substitutions based
on the functionality of the residue in the protein.

| Important Feature | Conservative Groupings |
|---|---|
| Charge/Polarity | 1. H, R, and K |
| | 2. D and E |
| | 3. C, T, S, G, N, Q, and Y |
| | 4. A, P, M, L, I, V, F, and W |
| Hydrophobicity | 1. D, E, N, Q, R, and K |
| | 2. C, S, T, P, G, H, and Y |
| | 3. A, M, I, L, V, F, and W |
| Structural/Surface Exposure | 1. D, E, N, Q, H, R, and K |
| | 2. C, S, T, P, A, G, W, and Y |
| | 3. M, I, L, V, and F |
| Secondary Structure Propensity | 1. A, E, Q, H, K, M, L, and R |
| | 2. C, T, I, V, F, Y, and W |
| | 3. S, G, P, D, and N |
| Evolutionary Conservation | 1. D and E |
| | 2. H, K, and R |
| | 3. N and Q |
| | 4. S and T |
| | 5. L, I, and V |
| | 6. F, Y, and W |
| | 7. A and G |
| | 8. M and C |

The terms “identical” or percent “identity,” in the context of two or more nucleic acids or peptide sequences, refer to two or more sequences or subsequences that are the same or have a specified percentage of amino acid residues or nucleotides that are the same (e.g., about 60% identity, preferably 65%, 70%, 75%, 80%, 85%, 90%, 91%, 92%, 93%, 94%, 95%, 96%, 97%, 9800, 9900, or higher identity over a specified region, when compared and aligned for maximum correspondence over a comparison window or designated region) as measured using a BLAST or BLAST 2.0 sequence comparison algorithms with default parameters described below, or by manual alignment and visual inspection.

As is known in the art, a number of different programs may be used to identify whether a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math., 2:482 (1981), by the sequence identity alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res., 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc, all of which are incorporated by reference.

An example of a useful algorithm is PILEUP. PILEUP creates a multiple sequence alignment from a group of related sequences using progressive, pair wise alignments. It may also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989), both incorporated by reference. Useful PILEUP parameters including a default gap weight of 3.00, a default gap length weight of 0.10, and weighted end gaps.

Another example of a useful algorithm is the BLAST algorithm, described in: Altschul et al., J. Mol. Biol. 215, 403-410, (1990); Altschul et al., Nucleic Acids Res. 25:3389-3402 (1997); and Karlin et al., Proc. Natl. Acad. Sci. U.S.A. 90:5873-5787 (1993), both incorporated by reference. A particularly useful BLAST program is the WU-BLAST-2 program which was obtained from Altschul et al., Methods in Enzymology, 266:460-480 (1996); http://blast.wustl/edu/blast/README.html]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST, as reported by Altschul et al., Nucl. Acids Res., 25:3389-3402, incorporated by reference. Gapped BLAST uses BLOSUM-62 substitution scores; threshold T parameter set to 9; the two-hit method to trigger ungapped extensions; charges gap lengths of k a cost of 10+k; Xu set to 16, and Xg set to 40 for database search stage and to 67 for the output stage of the algorithms. Gapped alignments are triggered by a score corresponding to ˜22 bits.

A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored). In a similar manner, “percent (%) nucleic acid sequence identity” with respect to the coding sequence of the polypeptides identified is defined as the percentage of nucleotide residues in a candidate sequence that are identical with the nucleotide residues in the coding sequence of the cell cycle protein. A preferred method utilizes the BLASTN module of WU-BLAST-2 set to the default parameters, with overlap span and overlap fraction set to 1 and 0.125, respectively.

As used herein, the term “coronavirus infection” refers to any infection, disease, disorder, or condition in a subject that is caused by an RNA virus in the group of RNA viruses classified as the family Coronaviridae. Coronaviruses are made up of a viral envelope and a nucleocapsid enclosing a positive-sense single-stranded RNA genome ranging from approximately 26 to 32 kilobases. The Coronaviridae family encompasses the Orthocoronavirinae and Letovirinae subfamilies. However, it is the Orthocoronavirinae subfamily, species of which are known to primarily infecte mammals and avians, that is of primary therapeutic interest, since species of the Letovirinae subfamily are only known to infect amphibians.

The Orthocoronavirinae subfamily emcompasses the alphacoronavirus, betacoronavirus, gammacoronavirus, and deltacoronavirus genuses. The alphacoronavirus and betacoronavirus are of primary therapeutic interest for the methods described herein. Examples of alphacoronavirus species include Alphacoronavirus 1 TGEV, Human coronavirus 229E (known to cause the common cold), Human coronavirus NL63 (known to cause the common cold), Miniopterus bat coronavirus 1, Miniopterus bat coronavirus HKU8, Porcine epidemic diarrhea virus, Rhinolophus bat coronavirus HKU2, and Scotophilus bat coronavirus 512. Non-limiting examples of betacoronavirus species include Betacoronavirus 1 species, e.g., Bovine Coronavirus, Human coronavirus OC43 (known to cause the common cold), Hedgehog coronavirus 1, Human coronavirus HKU1 (known to cause the common cold), Middle East respiratory syndrome-related coronavirus (known to cause MERS), Murine coronavirus MHV, Pipistrellus bat coronavirus HKU5, Rousettus bat coronavirus HKU9, Severe acute respiratory syndrome-related coronavirus species, e.g., SARS-CoV (known to cause SARS), SARS-CoV-2 (known to cause COVID-19), and Tylonycteris bat coronavirus HKU4. Non-limiting examples of gammacoronaviruses include Avian coronavirus IBV and Beluga whale coronavirus SW1. Non-limiting examples of deltacoronaviruses include Bulbul coronavirus HKU11 and Porcine coronavirus HKU15.

As used herein, the term “gene” refers to the segment of a DNA molecule that codes for a polypeptide chain (e.g., the coding region). In some embodiments, a gene is positioned by regions immediately preceding, following, and/or intervening the coding region that are involved in producing the polypeptide chain (e.g., regulatory elements such as a promoter, enhancer, polyadenylation sequence, 5′-untranslated region, 3′-untranslated region, or intron).

As used herein, the term “gene therapy” refers to any therapeutic approach of providing a nucleic acid (e.g., a polynucleotide) encoding a polypeptide (e.g., a protein and/or enzyme) to a subject to relieve, diminish, or prevent the occurrence of one or more symptoms of a disease (e.g., a coronavirus infection) and/or a condition associated with a deficiency or absence of the polypeptide in the subject. The term encompasses administering any compound, drug, procedure, or regimen comprising a Klotho polynucleotide encoding a Klotho polypeptide (e.g., an α-Klotho, β-Klotho, or γ-Klotho), including any modified form of a Klotho polynucleotide encoding any isoforms, variants, and/or recombinant Klotho polypeptides for maintaining the health of an individual with either the disease or the polypeptide deficiency. In some embodiments, gene therapy refers to the therapeutic insertion of an exogenous nucleic acid sequence into the genome of the subject by delivering the nucleic acid sequence into one or more cells of the subject. In some such embodiments, the exogenous polynucleotide is delivered by means of a vector capable of invading host cells and inserting genetic material into the host genome, such as a plasmid, nanostructure or virus. For example, in some embodiments, gene therapy is performed using a viral vector (e.g., a retrovirus, lentivirus, herpes virus, adenovirus, adeno-associated virus, and/or plasmid). The size of the exogenous nucleic acid to be inserted can vary depending on the type of vector used (ranging, for example, from less than 5 kilobases to greater than 30 kilobases or, in the case of plasmids, unlimited sizes). Alternate methods for gene editing include non-viral delivery systems, such as microinjections and other physical approaches that can be used to deliver allele-specific oligonucleotides (ASO), small interfering RNAs (siRNA), cationic polymers, cationic liposomes, and other nanoparticles. Gene therapy can also comprise CRISPR technology, which allows for Cas9-mediated targeted cleavage of the host genome and insertion of exogenous genetic material into the targeted region. In some embodiments, the gene therapy is administered by oral, intravenous, subcutaneous, and/or intramuscular means. In some embodiments, the gene therapy comprises administering a therapeutic composition comprising a therapeutically effective amount of a polynucleotide. See, for example, Goncalves and Paiva, 2017, “Gene therapy: advances, challenges and perspectives,” Einstein (Sao Paolo), 15(3): 369-375, doi: 10.1590/51679-45082017RB4024, which is hereby incorporated herein by reference in its entirety.

As used herein, the term “Klotho polypeptide” refers to any polypeptide with high sequence identity (e.g., at least 70%, 75%, 80%, 85%, 90%, 95%, 98%, 99%, or more) to the amino acid sequence of a wild type Klotho protein, e.g., an alpha-Klotho (α-klotho), beta-Klotho (0-klotho), or gamma-Klotho (γ-klotho) mature protein (inclusive of known isoforms and reduced constructs retaining significant wild type Klotho function, significant Klotho activity (e.g., at least 10%, 15%, 20%, 25%, or more of the corresponding wild type Klotho activity), or a polypeptide precursor of a Klotho protein thereof. Klotho proteins are believed to be a single pass transmembrane proteins located at the cell membrane that has also been detected in the Golgi apparatus. See, for example, Kuro-o et al., 1997, “Mutation of the mouse klotho gene leads to a syndrome resembling ageing,” Nature 390, 45-51; Matsumura et al., 1998, “Identification of the human klotho gene and its two transcripts encoding membrane and secreted klotho protein,” Biochem Biophys Res Commun 242, 626-630; Ito et al., 2000, “Molecular cloning and expression analyses of mouse betaklotho, which encodes a novel Klotho family protein,” Mech. Dev. 98:115-9; Shiraki-lida et al., 1998, “Structure of the mouse klotho gene and its two transcripts encoding membrane and secreted protein,” FEBS Lett Mar 424(1-2):6-10; and Imura et al., 2007, “α-Klotho as a Regulator of Calcium Homeostasis,” Science 316 (5831), 1615-1618. The human Klotho protein includes three subfamilies: alpha-Klotho (α-klotho), beta-Klotho (β-klotho), and gamma-Klotho (γ-klotho).

As used herein, the term “alpha Klotho polypeptide” or “α-Klotho polypeptide” refers to any polypeptide with high sequence identity (e.g., at least 70%, 75%, 80%, 85%, 90%, 95%, 98%, 99%, or more) to the amino acid sequence of a wild type alpha-Klotho (α-Klotho) mature protein (inclusive of known isoforms, soluble forms, and reduced constructs retaining significant wild type alpha Klotho function), significant alpha Klotho activity (e.g., at least 10%, 15%, 20%, 25%, or more of the corresponding wild type alpha Klotho activity), or a polypeptide precursor of a Klotho protein thereof. For instance, human full-length α-Klotho, alternately termed “Klotho,” is a 1012 amino acid residue, single pass type I transmembrane protein with an extracellular domain and a short cytoplasmic domain (SEQ ID NO:1, GenBank Accession No. NP004786). Other examples of wild type alpha Klotho polypeptides include NP_038851.2 (mouse), NP_001178124.1 (cow), and NP_112626.1 (rat).

The extracellular domain of human α-Klotho protein comprises two spherically-folded discrete subdomains termed KL1 (human residues 29-568, 540 residues long) and KL2 (human residues 569-980, 411 residues long). These two subdomains share amino acid sequence homology to β-glucosidase of bacteria and plants but lack glucosidase catalytic activity (Kuro-o et al., 1997). The N-terminus of the α-Klotho protein (residues 1-28) trails from KL1. The extracellular domain of the α-Klotho protein is bound to the cell surface by the transmembrane domain or is cleaved and released into the extracellular milieu. Membrane-bound α-Klotho protein is anchored in a cell membrane through the C-terminus (residues 981-1012). Alternately, in some embodiments, cleavage of the extracellular domain is facilitated by local low extracellular Ca² concentrations. Human α-Klotho protein exists in transmembrane, secreted, and soluble forms (e.g., obtained by alternative splicing and/or post-translational processing). For example, KL1-KL2 can be cleaved together to form a single 130 kDa secreted Klotho protein, also called soluble Klotho protein (residues 1-980), which is shed into the serum and acts as a circulating hormone (See, Imura et al., 2004, “Secreted Klotho protein in sera and CSF: implication for post-translational cleavage in release of Klotho protein from cell membrane,” FEBS Lett. May 7; 565(1-3):143-7). KL1 and KL2 can also be cleaved separately to form a 68 kDa protein and a 64 kDa protein, respectively.

In some embodiments, “Klotho activity” refers to any biological effect or activity exhibited by a Klotho protein or any variant thereof. For example, modulation of α-Klotho expression has been demonstrated to produce aging-related characteristics in mammals. Mice homozygous for a loss of function mutation in the α-Klotho gene develop characteristics resembling human aging, including shortened lifespan, skin atrophy, muscle wasting, arteriosclerosis, pulmonary emphysema and osteoporosis. In contrast, overexpression of the α-Klotho gene in mice extends lifespan and increases resistance to oxidative stress relative to wild-type mice. See, for example, M. Kuro-o et al., “Mutation of the mouse klotho gene leads to a syndrome resembling ageing,” Nature, 390 (1997), pp. 45-51; H. Kurosu et al., “Suppression of aging in mice by the hormone klotho,” Science, 309 (2005), pp. 1829-1833. α-Klotho acts as an essential factor for the specific interaction between FGF23 and FGFR1. Additionally, soluble α-Klotho protein has been implicated in a number of biological activities including a humoral factor that regulates activity of multiple glycoproteins on the cell surface, including ion channels and growth factor receptors such as insulin/insulin-like growth factor-1 receptors.

As used herein, the term “beta Klotho polypeptide” or “β-Klotho polypeptide” refers to any polypeptide with high sequence identity (e.g., at least 70%, 75%, 80%, 85%, 90%, 95%, 98%, 99%, or more) to the amino acid sequence of a wild type beta-Klotho (O-Klotho) mature protein (inclusive of known isoforms, soluble forms, and reduced constructs retaining significant wild type beta Klotho function), significant beta Klotho activity (e.g., at least 10%, 15%, 20%, 25%, or more of the corresponding wild type beta Klotho activity), or a polypeptide precursor of a beta Klotho protein thereof. For instance, human full-length β-Klotho is a 1044 amino acid residue, single pass type I transmembrane protein with extracellular KL1 and KL2 subdomains (SEQ ID NO:2, GenBank Accession No. NP783864). Other examples of wild type beta Klotho polypeptides include NP_112457.1 (mouse) and NP_001192255.1 (cow).

β-Klotho polypeptides can also include one or more of the intracellular, extracellular, and/or transmembrane domains of human β-Klotho, as well as any transmembrane, secreted, and/or soluble forms of β-Klotho (e.g., obtained by alternative splicing). For example, human (3-Klotho comprises an extracellular domain (residues 1-996), a transmembrane helical domain (residues 997-1017), and a cytoplasmic domain (residues 1018-1044). The KL1 and KL2 subdomains of the extracellular domain span residues 77-508 and 517-967, respectively. In some embodiments, the term “Klotho activity” refers to any biological effect or activity exhibited by a β-Klotho protein, including interaction with FGFR1 and FGFR4, direct interaction with FGF19, and/or direct interaction with FGF21 via the C-terminus of the protein.

As used herein, the term “gamma Klotho polypeptide” or “γ-Klotho polypeptide” refers to any polypeptide with high sequence identity (e.g., at least 70%, 75%, 80%, 85%, 90%, 95%, 98%, 99%, or more) to the amino acid sequence of a wild type gamma-Klotho (γ-Klotho) mature protein (inclusive of known isoforms, soluble forms, and reduced constructs retaining significant wild type gamma Klotho function), significant gamma Klotho activity (e.g., at least 10%, 15%, 20%, 25%, or more of the corresponding wild type gamma Klotho activity), or a polypeptide precursor of a gamma Klotho protein thereof. For instance, human full-length γ-Klotho, also known as KL lactase phlorizin hydrolase or lactase-like protein (LCTL), is a 567 amino acid residue, membrane protein that plays a role in the formation of the lens suture in the eye that is essential for normal optical properties of the lens. γ-Klotho polypeptides also include any one or more of the intracellular, extracellular, and/or transmembrane domains of human γ-Klotho, as well as any transmembrane, secreted, and/or soluble forms of γ-Klotho (e.g., obtained by alternative splicing). For example, human γ-Klotho comprises an extracellular domain (residues 23-541), a transmembrane helical domain (residues 542-562), and a cytoplasmic domain (residues 563-567) (SEQ ID NO:3, GenBank Accession No. NP_997221). Other examples of wild type beta Klotho polypeptides include XP_003121790.4 (pig), XP_001497077.2 (horse), and XP_001174693.1 (chimpanzee).

γ-Klotho polypeptides include any one or more of the intracellular, extracellular, and/or transmembrane domains of human γ-Klotho, as well as any transmembrane, secreted, and/or soluble forms of γ-Klotho (e.g., obtained by alternative splicing). For example, human γ-Klotho comprises an extracellular domain (residues 23-541), a transmembrane helical domain (residues 542-562), and a cytoplasmic domain (residues 563-567).

Non-limiting examples of wild-type Klotho protein include membrane-bound human α-Klotho isoform 1 (residues 1-1012); secreted human α-Klotho isoform 2 (residues 1-549); secreted human α-Klotho isoform 2 (residues 1-549) where the amino acid sequence differs from the canonical sequence at residues 535-549 (e.g., 535-549: DTTLSQFTDLNVYLW→SQLTKPISSLTKPYH); human γ-Klotho isoform 1 (residues 1-567); and/or human γ-Klotho isoform 2 (residues 174-567). Non-limiting examples of Klotho protein natural variants include α-Klotho natural variants (e.g., H193R, P15Q, F45V, H193R, F352V, C370S, P514S, P954L), β-Klotho natural variants (e.g., P65A, R728Q, A747V, Y906H, Q1020K), and γ-Klotho natural variants (e.g., T212M, A240T).

The term “Klotho polypeptide” can refer to a native or wild-type Klotho protein or a fragment, variant, analog or derivative thereof, e.g., a soluble form of the protein, or an active segment (e.g., of the native protein or of the extracellular domain), or any composition comprising a Klotho protein, fragment, variant, analog, derivative, or active segment thereof. Klotho proteins, including soluble forms, include but are not limited to α-Klotho, β-Klotho, γ-Klotho, and/or effective fragments thereof. The Klotho protein, fragment, variant, or derivative may be any suitable klotho protein, fragment, variant, or derivative and may be made, isolated, and purified in any suitable fashion with which one skilled in the art.

The term “Klotho polypeptide” is understood to include splice variants and fragments thereof retaining biological activity, and homologs thereof, having at least 70%, at least 75%, at least 80%, at least 85%, at least 90% at least 95%, or at least 99% homology thereto. In addition, this term is understood to encompass polypeptides resulting from minor alterations in the Klotho (e.g., alpha, beta, or gamma) coding sequence, such as, inter alia, point mutations, substitutions, deletions and insertions which may cause a difference in a few amino acids between the resultant polypeptide and the naturally occurring Klotho polypeptide. Polypeptides encoded by nucleic acid sequences which bind to the Klotho coding sequence or genomic sequence under conditions of highly stringent hybridization, which are well-known in the art are also encompassed by this term. Chemically-modified Klotho polypeptide or chemically-modified fragments of Klotho polypeptide are also included in the term, so long as the biological activity is retained. See, for example, PCT publication WO2011084452A1, “Therapeutic uses of soluble alpha-klotho,” for further details regarding soluble α-Klotho, and PCT publication WO2017085317A1, “Secreted splicing variant of mammal klotho as a medicament for cognition and behaviour impairments,” for further details regarding secreted splicing variants of Klotho proteins, each of which is hereby incorporated herein by reference in its entirety.

It is acknowledged that differences in the amino acid sequence can exist among various tissues of an organism and among different organisms of one species or among different species to which the nucleic acid according to the present invention can be applied in various embodiments of the present invention. The term “Klotho polypeptide” is understood to include a polypeptide including an amino acid sequence having a high sequence identity (e.g., at least 70%, 75%, 80%, 85%, 90%, 95%, 98%, 99%, or more) to the amino acid sequence of Klotho protein (e.g., alpha, beta, and/or gamma) obtained from one or more diverse tissues in a human (e.g., serum, cerebrospinal fluid, kidney, pancreas, placenta, small intestine, prostate, renal cell carcinomas, hepatocellular carcinomas, retina, lung, stomach, esophagus, spleen, heart, smooth muscle, epithelium, brain, colon, bladder, and/or thyroid, among others). See, for example, U.S. Patent No. US20120178699A1, “Klotho protein and related compounds for the treatment and diagnosis of cancer,” which is hereby incorporated herein by reference in its entirety, for further details regarding Klotho amino acid sequences obtained from different tissues and organisms.

The term “Klotho polypeptide” is understood to include particular fragments of the human Klotho polypeptide such as amino acid residues 29-1012, 1-980, 29-980, 31-982, 34-1012, 1-568, 29-568, 34-549, and/or 29-549 of wild-type α-Klotho (SEQ ID NO:1, GenBank Accession No. NP004786). In some embodiments, the Klotho polypeptide has a sequence identity of at least 70%, 75%, 80%, 85%, 90%, 95%, 98%, 99%, or more to amino acid residues 29-1012, 1-980, 29-980, 31-982, 34-1012, 1-568, 29-568, 34-549, and/or 29-549 of wild-type α-Klotho (SEQ ID NO:1).

According to some embodiments, the Klotho polypeptide is a pegylated Klotho protein (e.g., alpha, beta, and/or gamma), for example, a protein substantially similar or identical to Klotho proteins described herein that has been pegylated to improve pharmacokinetics or other parameters. Various advantages of pegylation and methods for pegylation of proteins such as Klotho proteins are known in the art. See, for example, Ryan et al., 2008, “Advances in PEGylation of important biotech molecules: delivery aspects,” Expert Opin Drug Deliv. 5(4), 371-383.

The term “Klotho polypeptide” is understood to include a variant Klotho polypeptide having one or more sequence substitutions, deletions, and/or additions as compared to the native sequence. In some embodiments, a variant Klotho polypeptide is artificially constructed (e.g., generated from corresponding nucleic acid molecules). In some embodiments, the variant Klotho polypeptide has 1 or 2 amino acid substitutions and retains at least some of the activity of the native polypeptide. Examples of variant Klotho polypeptides include, without limitation, a polypeptide comprising an amino acid sequence for α-Klotho, β-Klotho, or γ-Klotho (e.g., SEQ ID NOS: 1, 2, or 3) where at least one amino acid of the amino acid sequence is deleted, substituted or added. See, for example, U.S. Patent No. US20120178699A1, “Klotho protein and related compounds for the treatment and diagnosis of cancer,” which is hereby incorporated herein by reference in its entirety. In some embodiments, a variant Klotho polypeptide is a polypeptide comprising an amino acid sequence for α-Klotho, β-Klotho, or γ-Klotho (e.g., SEQ ID NOS: 1, 2, or 3) and having at least one amino acid mutation in the catalytic domain of the respective Klotho protein. In some embodiments, a variant Klotho polypeptide is a polypeptide comprising an amino acid sequence for α-Klotho (e.g., SEQ ID NO:1), where the L-Glu of residue 414 is substituted with an R-α-amino acid residue, an L-α-amino acid residue different from L-Glu (e.g., Ala, Arg, Asn, Asp, Cys, Gln, Gly, His, Ile, Leu, Lys, Met, Phe, Pro, Ser, Thr, Trp, Tyr, Val, ornithine, selenocysteine (Sec), 2-aminoisobutyric acid, hydroxyproline (Hyp) and selenomethionine), and/or an α-amino acid residue that is devoid of an acid side chain (e.g., L-α-Gln). In some embodiments, a variant Klotho polypeptide is a polypeptide comprising an amino acid sequence for α-Klotho (e.g., SEQ ID NO:1), where the L-Asp of residue 238 is substituted with an R-α-amino acid residue, an L-α-amino acid residue different from L-Asp (e.g., Ala, Arg, Asn, Cys, Gln, Glu, Gly, His, Ile, Leu, Lys, Met, Phe, Pro, Ser, Thr, Trp, Tyr, Val, ornithine, selenocysteine (Sec), 2-aminoisobutyric acid, hydroxyproline (Hyp) and selenomethionine), and/or an α-amino acid residue that is devoid of an acid side chain (e.g., L-α-Asn). In some embodiments, a variant Klotho polypeptide is a polypeptide comprising an amino acid sequence for α-Klotho (e.g., SEQ ID NO:1) having the mutation Glu414Gln and/or Asp238Asn. See, for example, U.S. Patent No. US20150079065A1, “Klotho variant polypeptides and uses thereof in therapy,” which is hereby incorporated herein by reference in its entirety.

In some embodiments, the variant Klotho polypeptide is encoded by a variant Klotho polynucleotide, where at least one nucleotide base of the nucleic acid sequence is deleted, substituted or added. Non-limiting examples of variant Klotho polynucleotides include a polynucleotide that encodes α-Klotho comprising: a cytosine at nucleotide position 1122; a deleted adenine at nucleotide position 1337; a guanine at nucleotide position 1686; a guanine at nucleotide position 2406; a cytosine at nucleotide position 12707; an adenine at nucleotide position 12753; a cytosine at nucleotide position 19489; a thymine at nucleotide position 19969; and/or a thymine at nucleotide position 20445. See, for example, PCT publication WO2001020031A2, “Polymorphisms in a klotho gene,” which is hereby incorporated herein by reference in its entirety.

The term “Klotho polypeptide” is understood to include recombinant or fusion Klotho polypeptides, such as a native Klotho amino acid sequence modified with a water-soluble polypeptide. In some embodiments, a recombinant Klotho polypeptide is chemically or enzymatically modified (e.g., PEG, polysialic acid, and/or hydroxyethyl starch). In some embodiments, the modification is performed in-vitro. In some embodiments, the recombinant Klotho polypeptide is a fusion protein with a half-life extending peptide moiety (e.g., an Fc domain, albumin polypeptide, albumin-binding peptide, and/or XTEN peptide).

In some embodiments, the term “Klotho polypeptide” refers to a human polypeptide variant having identity or homology of at least 70%, at least 75%, at least 80%, at least 85%, at least 90%, at least 91%, at least 92%, at least 93%, at least 94%, at least 95%, at least 96%, at least 97%, at least 98%, or at least 99% to at least one or more native or wild-type Klotho protein or a fragment, variant, analog or derivative thereof. In some embodiments, the term “Klotho polypeptide” refers to a nonhuman polypeptide variant having identity or homology of at least 70%, at least 75%, at least 80%, at least 85%, at least 90%, at least 91%, at least 92%, at least 93%, at least 94%, at least 95%, at least 96%, at least 97%, at least 98%, or at least 99% to at least one or more native or wild-type Klotho protein or a fragment, variant, analog or derivative thereof. Non-limiting examples of nonhuman Klotho polypeptides include murine, primate, bovine, canine or equine forms, including any forms obtained from one or more different tissues of such organisms. See, PCT publication WO2014152993A1, “Use of klotho nucleic acids or proteins for treatment of diabetes and diabetes-related conditions,” which is hereby incorporated herein by reference in its entirety.

In some embodiments, Klotho polypeptides in a biological sample are analyzed using any method for polypeptide detection and/or measurement known to one skilled in the art. For example, in some embodiments, Klotho polypeptides are quantitatively analyzed using immunodetection. In some such embodiments, Klotho polypeptides are analyzed using an immunodetection kit such as enzyme-linked immunosorbent assay (ELISA) (e.g., LifeSpan BioSciences KLB/Beta Klotho ELISA Kit, Biomatik Human Klotho ELISA Kit, IBL America Alpha-Klotho Soluble ELISA Kit, and/or Aviva Systems Biology Human KL Chemi-Luminescent ELISA Kit).

Klotho polypeptides include Klotho polypeptides obtained from a manufacturer or supplier (e.g., recombinant Klotho polypeptides, native Klotho polypeptides, Klotho polypeptide lysates, chimeric Klotho polypeptides, and/or human Klotho polypeptide expressed in E. coli or mammalian cells), as well as Klotho polypeptides recovered from source biologic tissue, e.g., human plasma samples. Commercial suppliers of Klotho polypeptides include, e.g., GeneTex, LifeSpan BioSciences, Novus Biologicals, Biorbyt, Abcam, BioVision, Origene, and PeproTech.

As used herein, the term “Klotho polynucleotide” refers to a nucleic acid sequence that encodes a Klotho polypeptide, where the Klotho polypeptide is any of the embodiments detailed herein. As used herein, the term “Klotho gene” refers to a Klotho polypeptide coding sequence open reading frame or any homologous sequence thereof having at least 70%, at least 75%, at least 80%, at least 85%, at least 90%, at least 95%, at least 98%, or at least 99% identity. This encompasses nucleic acid sequences that have undergone mutations, alterations or modifications as described herein, and/or nucleic acid sequences that have been mutated, altered, or modified to encode any of the Klotho polypeptides and/or variant Klotho polypeptides described herein. It is also to be acknowledged that based on the amino acid sequence of a Klotho polypeptide or variants described herein, any nucleic acid sequence coding for such amino acid sequence can be perceived by one skilled in the art based on the genetic code. It is to be understood that the term “Klotho polynucleotide” includes any nucleic acid sequence encompassing, for example, known nucleotide analogs or modified backbone residues or linkages, which are synthetic, naturally occurring, and/or non-naturally occurring (e.g., DNA, RNA, and/or cDNA).

As used herein, the term “nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

As used herein, the term “polypeptide treatment” refers to any therapeutic approach of providing a polypeptide (e.g., a protein and/or enzyme) to a subject to relieve, diminish, or prevent the occurrence of one or more symptoms of a disease (e.g., a coronavirus infection) and/or a condition associated with a deficiency or absence of the polypeptide in the subject. The term encompasses administering any compound, drug, procedure, or regimen comprising a Klotho polypeptide (e.g., an α-Klotho, β-Klotho, or γ-Klotho), including any modified form of a Klotho polypeptide such as any isoforms, variants, and/or recombinant Klotho polypeptides for maintaining the health of an individual with either the disease or the polypeptide deficiency. In some embodiments, the polypeptide treatment is administered by oral, intravenous, subcutaneous, and/or intramuscular means. In some embodiments, the polypeptide treatment comprises administering a therapeutic composition comprising a therapeutically effective amount of a polypeptide, such as a protein or an enzyme. See, for example, Safary et al., 2018, “Enzyme replacement therapies: what is the best option?” Bioimpacts 8(3): 153-157; doi: 10.15171/bi.2018.17, which is hereby incorporated herein by reference in its entirety.

As used interchangeably herein, the term “treatment” or “therapy” generally means obtaining a desired physiologic effect. The effect may be prophylactic in terms of completely or partially preventing a disease or condition or symptom thereof and/or may be therapeutic in terms of a partial or complete cure for an injury, disease, or condition and/or amelioration of an adverse effect attributable to the injury, disease or condition and includes arresting the development or causing regression of a disease or condition. The effects may be a delay in onset, amelioration of symptoms, improvement in patient survival, increase in survival time or rate, improvement in cognitive function, etc. The effect may be improved health following eradication of the disease condition, e.g., a lessining of lasting effects caused by the disease and/or long-term complications resulting from the disease or condition (e.g., during or after the partial or complete cure for the disease or condition). The effect of treatment can be compared to an individual or pool of individuals not receiving the treatment.

As used interchangeably herein, a “therapeutically effective amount or dose” or “sufficient/effective amount or dose,” refers to a dose that produces effects for which it is administered. The exact dose will depend on the purpose of the treatment, and will be ascertainable by one skilled in the art using known techniques (See, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins, the disclosures of which are herein incorporated by reference in their entireties for all purposes). As used here, the terms “dose” and “dosage” are used interchangeably and refer to the amount of active ingredient given to an individual at each administration. The dose will vary depending on a number of factors, including frequency of administration; size and tolerance of the individual; severity of the condition; risk of side effects; and the route of administration. One of skill in the art will recognize that the dose can be modified depending on the above factors or based on therapeutic progress. The term “dosage form” refers to the particular format of the pharmaceutical, and depends on the route of administration. For example, a dosage form can be a liquid, formulated for administration via intravenous infusion and/or subcutaneous injection.

As used herein, a therapeutic composition refers to a mixture of components for therapeutic administration. In some embodiments, a therapeutic composition comprises a therapeutically active agent and one or more of a buffering agent, solvent, nanoparticle, microcapsule, viral vector and/or other stabilizers. In some embodiments, the therapeutically active agent is, for example, a Klotho polypeptide and/or a Klotho polynucleotide that encodes a Klotho polypeptide. In some embodiments, a therapeutic composition may also contain residual levels of chemical agents used during the manufacturing process, e.g., surfactants, buffers, salts, and stabilizing agents, as well as chemical agents used to pH the final composition, for example, counter ions contributed by an acid (e.g., hydrochloric acid or acetic acid) or base (e.g., sodium or potassium hydroxide), and/or trace amounts of contaminating proteins.

As used herein, the term “vector” refers to any vehicle used to transfer a nucleic acid (e.g., encoding a Klotho polypeptide) into a host cell. In some embodiments, a vector includes a replicon, which functions to replicate the vehicle, along with the target nucleic acid. Non-limiting examples of vectors useful for gene therapy include plasmids, phages, cosmids, artificial chromosomes, and viruses, which function as autonomous units of replication in vivo. In some embodiments, a vector is a viral vehicle for introducing a target nucleic acid (e.g., a codon-altered polynucleotide encoding a Klotho polypeptide). Many modified eukaryotic viruses useful for gene therapy are known in the art. For example, adeno-associated viruses (AAVs) are particularly well suited for use in human gene therapy because humans are a natural host for the virus, the native viruses are not known to contribute to any diseases, and the viruses illicit a mild immune response.

Before the present disclosure is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Diseases Caused by Coronaviruses

As described above, in some embodiments, a disease caused by a coronavirus is caused by, characterized by, or associated with an alphacoronavirus (e.g., Alphacoronavirus 1 TGEV, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, and/or Scotophilus bat coronavirus 512), a betacoronavirus (e.g., Betacoronavirus 1 (Bovine Coronavirus, Human coronavirus OC43), Hedgehog coronavirus 1, Human coronavirus HKU1, Middle East respiratory syndrome-related coronavirus, Murine coronavirus MHV, Pipistrellus bat coronavirus HKU5, Rousettus bat coronavirus HKU9, Severe acute respiratory syndrome-related coronavirus (SARS-CoV, SARS-CoV-2), and/or Tylonycteris bat coronavirus HKU4), a gammacoronavirus (e.g., Avian coronavirus IBV and/or Beluga whale coronavirus SW1), or a deltacoronavirus (e.g., Bulbul coronavirus HKU11 and/or Porcine coronavirus HKU15). In some embodiments, a coronavirus infection is caused by transmission of a coronavirus via an aerosol, fomite, or fecal-oral route.

In some embodiments, a disease caused by a coronavirus is caused by, characterized by, or associated with a human-infective coronavirus, including Human coronavirus OC43 (HCoV-OC43), Human coronavirus HKU1 (HCoV-HKU1), Human coronavirus 229E (HCoV-229E), Human coronavirus NL63 (HCoV-NL63), Middle East respiratory syndrome-related coronavirus (MERS-CoV), Severe acute respiratory syndrome coronavirus (SARS-CoV or SARS-CoV-1), and/or Severe acute respiratory syndrome coronavirus 2 (SARS-CoV-2).

Severe Acute Respiratory Syndrome Coronavirus (SARS-CoV-1)

Severe acute respiratory syndrome (SARS) is a viral respiratory disease caused by SARS-CoV-1, a strain of severe acute respiratory syndrome-related coronavirus (SARSr-CoV). SARS-CoV-1, the causative agent of SARS, is primarily transmitted via contact of mucous membranes with respiratory droplets (e.g., coughing or sneezing) or with contaminated surfaces, after which the virus infects human epithelial cells within the lungs by binding to angiotensin-converting enzyme 2 (ACE2).

Humans infected with SARS-CoV-1 can develop fever (e.g., above 38° C. or 100° F.), muscle pain, lethargy, cough, sore throat, headache, and other flu-like symptoms, as well as shortness of breath and/or pneumonia (e.g., direct viral pneumonia or secondary bacterial pneumonia). In some cases, infected individuals can also present with decreased levels of circulating lymphocytes. In addition, long-term pathological conditions have been observed following the acute phase of the disease, including pulmonary fibrosis, osteoporosis, and femoral necrosis. Mortality ranges from 0% to 50% depending on age, with an overall case fatality rate of 11%.

Risk factors that can increase the chance of mortality include age and gender, with a mortality rate of 1% in patients under 24 compared to a mortality rate of over 55% in patients 65 and older, and a greater number of males succumbing to the disease compared to females.

Middle East Respiratory Syndrome-Related Coronavirus (MERS-CoV)

Middle East respiratory syndrome (MERS), or camel flu, is a viral respiratory disease caused by MERS-CoV, a coronavirus known to infect humans, camels, and bats. The causative agent is thought to be transmitted through inhalation of respiratory droplets during close contact with an infected individual, or through contact with infected camels and/or camel-based food products. Similar to SARS-CoV-1, MERS-CoV belongs to the gene betacoronavirus, and includes two genetically distinct clades (Clade A and B). In humans, the virus is thought to preferentially target nonciliated bronchial epithelial cells, evade the innate immune response and antagonize interferon production. Invasion occurs through binding to dipeptidyl peptidase 4 (DPP4, alternately CD26) on the surface of human bronchial epithelium and kidney cells, which act as a functional receptor for MERS-CoV.

Humans infected with MERS-CoV may be asymptomatic or may present with symptoms similar to those observed in SARS infections. These include fever, cough, expectoration, shortness of breath, and muscle pain. Other symptoms include gastrointestinal symptoms such as diarrhea, vomiting, abdominal pain. Severe cases also result pneumonia leading to acute respiratory distress syndrome, kidney failure, disseminated intravascular coagulation, and pericarditis. In some instances, infected individuals require mechanical ventilation. Mortality occurs in approximately 30% of cases, with roughly three times as many males succumbing to the disease compared to females.

Severe Acute Respiratory Syndrome Coronavirus 2 (SARS-CoV-2)

Coronavirus disease 2019 (COVID-19) is an infectious disease caused by SARS-CoV-2, a strain of SARSr-CoV. SARS-CoV-2 is thought to be transmitted between individuals by inhalation or contact with respiratory droplets (e.g., coughing, sneezing, and/or talking) or through contact with contaminated surfaces. The virus has been reported to preferentially target angiotensin-converting enzyme 2 (ACE2)-expressing epithelial cells in the respiratory tract, although the exact mechanism of action is unknown. Patients with severe COVID-19 exhibit symptoms of systemic hyperinflammation, including elevated IL-2, IL-7, IL-6, granulocyte-macrophage colony-stimulating factor (GM-CSF), interferon-7 inducible protein 10 (IP-10), monocyte chemoattractant protein 1 (MCP-1), macrophage inflammatory protein 1-α (MIP-1α), and tumour necrosis factor-α (TNF-α), as well as serum biomarkers of cytokine release syndrome (CRS) such as elevated C-reactive protein (CRP), lactate dehydrogenase (LDH), D-dimer, and ferritin.

SARS-CoV-2 infections vary widely, ranging from asymptomatic infections to mild or severe symptoms including fever, cough, fatigue, shortness of breath, muscle pain, nausea, vomiting, diarrhea, flu-like symptoms, loss of smell and taste, acute respiratory distress syndrome, cytokine storm, multi-organ failure, stroke, septic shock, blood clots, and/or death, among others. Additionally, a diversity of risk factors exists for complications, symptoms and health outcomes that the virus can exhibit and cause in infected patients. For example, risk factors for complications include gender, advanced age and health conditions that tend to be more prevalent in the elderly, such as hypertension, diabetes, obesity, COPD, cancer, chronic kidney disease, and smoking, among others. See, Blagosklonny, 2020, “From causes of aging to death from COVID-19,” Aging, 12 (11), 10004-10021, which is hereby incorporated herein by reference in its entirety.

Like SARS-CoV-1 and MERS-CoV, SARS-CoV-2 is a betacoronavirus. It shares 96% sequence identity to bat coronaviruses BatCov RaTG13 in the same subgenus. Notably, SARS-CoV-2 comprises a polybasic cleavage site that reportedly contributes to increased pathogenicity and transmissibility. See, Walls et al., 2020, “Structure, function and antigenicity of the SARS-CoV-2 spike glycoprotein,” Cell. 181 (2): 281-292.e6, doi:10.1016/j.cell.2020.02.058, and Coutard et al., 2020, “The spike glycoprotein of the new coronavirus 2019-nCoV contains a furin-like cleavage site absent in CoV of the same clade,” Antiviral Research. 176: 104742, doi:10.1016/j.antiviral.2020.104742, each of which is hereby incorporated herein by reference in its entirety. Entry of SARS-CoV-2 into host cells is thought to occur via a transmembrane protease that primes a structural protein (e.g., the spike protein) located on the viral envelope for binding to the host cell receptors. After attachment of SARS-CoV-2 to a host cell via the S1 subunit of the spike protein, the transmembrane protease serine 2 (TMPRSS2) cleaves the spike protein to expose a fusion peptide in the S2 subunit, allowing fusion with the host receptor (e.g., ACE2). See, Hoffman et al., 2020, “SARS-CoV-2 Cell Entry Depends on ACE2 and TMPRSS2 and Is Blocked by a Clinically Proven Protease Inhibitor,” Cell. 181 (2): 271-280.e8, doi:10.1016/j.cell.2020.02.052.

### Administration

In some embodiments, an effective amount of a polypeptide treatment and/or gene therapy is administered to the subject by any suitable means to treat the disease or disorder. For example, in certain embodiments, the polypeptide treatment and/or gene therapy may be administered by intravenous, intraocular, subcutaneous, and/or intramuscular means. The polypeptide treatment and/or gene therapy can be administered by parenteral (including intravenous, intradermal, intraperitoneal, intramuscular and subcutaneous) routes or by other delivery routes, including oral, nasal, buccal, sublingual, intra-tracheal, transdermal, transmucosal, and pulmonary. In certain embodiments, the polypeptide treatment and/or gene therapy provided herein can be administered either systemically or locally (e.g., directly).

Systemic administration includes: oral, transdermal, subdermal, intraperitioneal, subcutaneous, transnasal, sublingual, or rectal. Alternatively, the polypeptide treatment and/or gene therapy may be delivered via a sustained delivery device implanted, for example, subcutaneously or intramuscularly. The polypeptide treatment and/or gene therapy can be administered by continuous release or delivery, using, for example, an infusion pump, continuous infusion, controlled release formulations utilizing polymer, oil or water insoluble matrices.

In certain embodiments, the term “effective amount” refers to an amount of a polypeptide treatment and/or gene therapy that results in an improvement or remediation of disease or condition in the subject. An effective amount to be administered to the subject can be determined by a physician with consideration of individual differences in age, weight, the disease or condition being treated, disease severity and response to the therapy. In certain embodiments, the polypeptide treatment and/or gene therapy can be administered to a subject alone or in combination with other compositions. In some embodiments, the polypeptide treatment and/or gene therapy is administered at periodic intervals, over multiple time points, and/or for a duration of treatment. For example, in some such embodiments, the polypeptide treatment and/or gene therapy is administered at least every 1, 2, 3, 4, 6, 8, 12, or 24 hours, at least every 1, 2, 3, 4, 5, 6, or 7 days, at least every 1, 2, 3 or 4 weeks, or at least at a monthly, bi-monthly, annually or bi-annually frequency. In some embodiments, the polypeptide treatment and/or gene therapy is administered at a single time point. In some embodiments, the time needed to complete a course of the treatment is determined by a physician. In some embodiments, the course of treatment ranges from as short as one day to more than a month. In certain embodiments, a course of treatment can be from 1 to 6 months, or more than 6 months.

According to some embodiments, the polypeptide treatment and/or gene therapy is administered in extended release form, which is capable of releasing the protein over a predetermined release period, such that a therapeutically effective plasma level of the polypeptide treatment and/or gene therapy is maintained for at least 24 hours, such as at least 48 hours, at least 72 hours, at least one week, or at least one month.

In some embodiments, the polypeptide treatment and/or gene therapy comprises a formulation that is selected for the mode of delivery, e.g., intravenous, intraocular, subcutaneous, and/or intramuscular means.

According to some embodiments of the present invention, the polypeptide treatment and/or gene therapy can be administered in combination with one or more active therapeutic agents for treating co-infections or associated complications.

Where the treatment is a gene therapy (e.g., comprising therapeutically effective amount of a Klotho polynucleotide), the treatment can comprise, for example, a construct comprising the therapeutic agent (e.g., the Klotho polynucleotide), a vector comprising the therapeutic agent (e.g., the Klotho polynucleotide), a plasmid comprising the therapeutic agent (e.g., the Klotho polynucleotide), and/or a host cell comprising the therapeutic agent (e.g., the Klotho polynucleotide). In some embodiments, the gene therapy comprises a recombinant vector suitable for gene therapy (e.g., an adeno-associated virus, adenovirus, nanoparticle, plasmid, and/or lentivirus).

In some embodiments, the polypeptide treatment and/or gene therapy comprises a formulation that includes carriers, stabilizers, diluents, adjuvents and/or other excipients. Carriers or excipients known in the art can also be used to facilitate administration of the polypeptide treatment and/or gene therapy. Examples of carriers and excipients include calcium carbonate, calcium phosphate, various sugars such as lactose, or types of starch, cellulose derivatives, gelatin, vegetable oils, polyethylene glycols and physiologically compatible solvents.

Pharmaceutically acceptable carriers include sterile liquids, such as water and oils, including those of petroleum, animal, vegetable or synthetic origin, such as peanut oil, soybean oil, mineral oil, sesame oil and the like. For example, in some embodiments, water is a preferred carrier when the pharmaceutical composition is administered subcutaneously. Saline solutions and aqueous dextrose and glycerol solutions can also be employed as liquid carriers, particularly for injectable solutions.

If desired, solutions of the above compositions may be thickened with a thickening agent such as methylcellulose. In some embodiments, solutions are prepared in emulsified form, such as either water in oil or oil in water. Any of a wide variety of pharmaceutically acceptable emulsifying agents can be employed including, for example, acacia powder, a non-ionic surfactant (such as a Tween), or an ionic surfactant (such as alkali polyether alcohol sulfates or sulfonates, e.g., a Triton).

In general, the composition of the present invention is prepared by mixing the ingredients following generally accepted procedures. For example, the selected components can be simply mixed in a blender or other standard device to produce a concentrated mixture which may then be adjusted to the final concentration and viscosity by the addition of water or thickening agent and possibly a buffer to control pH or an additional solute to control tonicity.

### Klotho Polypeptide Treatment for Coronavirus Infection

Alpha-Klotho Polypeptide Treatment for Coronavirus Infection

In one aspect, the disclosure provides a method for treating a coronavirus infection by administering a Klotho polypeptide to a subject in need thereof, e.g., a subject infected by a coronavirus. In some embodiments, the treatment includes administration of an alpha-Klotho polypeptide to the subject. In some embodiments, the treatment includes administration of a beta-Klotho polypeptide to the subject. In some embodiments, the treatment includes administration of a gamma-Klotho polypeptide to the subject.

In some embodiments, the disclosure provides a method for treating an alphacoronavirus infection by administering a Klotho polypeptide to a subject in need thereof, e.g., a subject infected by an alphacoronavirus. In some embodiments, the treatment includes administration of an alpha-Klotho polypeptide to the subject. In some embodiments, the treatment includes administration of a beta-Klotho polypeptide to the subject. In some embodiments, the treatment includes administration of a gamma-Klotho polypeptide to the subject. In some embodiments, the alphavirus infection is an infection of the Human coronavirus 229E, known to cause the common cold. In some embodiments, the alphavirus infection is an infection of the Human coronavirus NL63, known to cause the common cold. Accordingly, in some embodiments, the disclosure provides a method for treating a cold comprising administering a Klotho polypeptide to a subject in need thereof, e.g., a subject with a cold.

In some embodiments, the disclosure provides a method for treating a betacoronavirus infection by administering a Klotho polypeptide to a subject in need thereof, e.g., a subject infected by a betacoronavirus. In some embodiments, the treatment includes administration of an alpha-Klotho polypeptide to the subject. In some embodiments, the treatment includes administration of a beta-Klotho polypeptide to the subject. In some embodiments, the treatment includes administration of a gamma-Klotho polypeptide to the subject. In some embodiments, the betacoronavirus infection is an infection of the Human coronavirus OC43, known to cause the common cold. In some embodiments, the betacoronavirus infection is an infection of the Human coronavirus HKU1, known to cause the common cold. Accordingly, in some embodiments, the disclosure provides a method for treating a cold comprising administering a Klotho polypeptide to a subject in need thereof, e.g., a subject with a cold. In some embodiments, the betacoronavirus infection is an infection of the Middle East respiratory syndrome-related coronavirus, known to cause MERS. Accordingly, in some embodiments, the disclosure provides a method for treating MERS comprising administering a Klotho polypeptide to a subject in need thereof, e.g., a subject with MERS. In some embodiments, the betacoronavirus infection is an infection of Severe acute respiratory syndrome-related coronavirus species, e.g., SARS-CoV, known to cause SARS. Accordingly, in some embodiments, the disclosure provides a method for treating SARS comprising administering a Klotho polypeptide to a subject in need thereof, e.g., a subject with SARS. In some embodiments, the betacoronavirus infection is an infection of SARS-CoV-2 (known to cause COVID-19). Accordingly, in some embodiments, the disclosure provides a method for treating COVID-19 comprising administering a Klotho polypeptide to a subject in need thereof, e.g., a subject with COVID-19.

In some embodiments, the coronavirus infection is caused by a human-infective coronavirus, including Human coronavirus OC43 (HCoV-OC43), Human coronavirus HKU1 (HCoV-HKU1), Human coronavirus 229E (HCoV-229E), Human coronavirus NL63 (HCoV-NL63), Middle East respiratory syndrome-related coronavirus (MERS-CoV), Severe acute respiratory syndrome coronavirus (SARS-CoV, alternately SARS-CoV-1), and/or Severe acute respiratory syndrome coronavirus 2 (SARS-CoV-2). The symptoms caused by human-infective coronaviruses range in type and severity, including fever, sore throat, pneumonia, bronchitis, and/or upper and lower respiratory tract infections. Typically, MERS-CoV, SARS-CoV-1 and SARS-CoV-2 produce symptoms that are potentially severe and in some cases can result in fatality in more than 30% of those infected.

In some embodiments, the coronavirus infection is caused by a severe acute respiratory syndrome-related coronavirus (SARSr-CoV). For example, SARS-CoV-1 and SARS-CoV-2 are human-infective strains of SARSr-CoV. SARSr-CoV strains also include those primarily found to infect non-human species, such as bats and palm civets. SARSr-CoV coronaviruses are members of the group of betacoronaviruses. Although SARSr-CoV shares a set of conserved domains with other betacoronaviruses, it comprises only a single papain-like proteinase (PLpro) instead of two in the open reading frame ORF1.

In some embodiments, the coronavirus infection is caused by SARS-CoV-1. SARS-CoV-1 is a strain of coronavirus that causes severe acute respiratory syndrome (SARS), characterized by often severe illness, systemic muscle pain, headache and fever, decreased levels of circulating lymphocytes, and respiratory symptoms including cough, dyspnea, and pneumonia.

In some embodiments, a coronavirus infection is caused by, characterized by, or associated with SARS-CoV-2. SARS-CoV-2 is a strain of coronavirus that causes coronavirus disease 2019 (COVID-19, alternately hCoV-19), a respiratory illness characterized by fever, cough, fatigue, shortness of breath, loss of smell and taste, acute respiratory distress syndrome, cytokine storm, multi-organ failure, septic shock, and/or blood clots, among others.

In some embodiments, a coronavirus infection is caused by an RNA virus sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to a strain of SARS-CoV-1. In some embodiments, a coronavirus infection is caused by, characterized by, or associated with an RNA virus sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to a strain of MERS-CoV (including, e.g., Clade A or Clade B). In some embodiments, a coronavirus infection is caused by, characterized by, or associated with an RNA virus sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to a strain of SARS-CoV-2.

One aspect of the present disclosure provides a method for treating a severe acute respiratory syndrome-related coronavirus (SARS-CoV) infection in a subject in need thereof, the method comprising administering a therapeutically effective amount of a Klotho polypeptide to the subject.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the present disclosure provides a method for treating a coronavirus infection, where the coronavirus infection is a Middle East respiratory syndrome coronavirus (MERS-CoV) infection. In some embodiments, the subject has been diagnosed with MERS or camel flu.

In some embodiments, the Klotho polypeptide is an α-Klotho polypeptide. In some embodiments, the α-Klotho polypeptide is any of the embodiments described herein (e.g., see Definitions: Klotho polypeptide). For example, in some embodiments, the α-Klotho polypeptide comprises a KL1 glycosyl hydrolase-1 domain and a KL2 glycosyl hydrolase-2 domain. In some alternative embodiments, the α-Klotho polypeptide comprises a KL1 glycosyl hydrolase-1 domain, but not a KL2 glycosyl hydrolase-2 domain.

In some embodiments, the α-Klotho polypeptide is a human α-Klotho polypeptide. In some embodiments, the human α-Klotho polypeptide comprises an amino acid sequence having at least 95% identity or at least 99% identity to amino acids 34-981 of SEQ ID NO:1 (the full-length, wild-type sequence of the human Klotho precursor protein—NP004786). In some embodiments, the human α-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 34-981 of SEQ ID NO:1. In some embodiments, the human α-Klotho polypeptide comprises an amino acid sequence of amino acids 34-981 of SEQ ID NO:1.

In some embodiments, the human α-Klotho polypeptide comprises an amino acid sequence having at least 95% identity or at least 99% identity to amino acids 34-549 of SEQ ID NO:1 (the full-length, wild-type sequence of the human Klotho precursor protein—NP004786). In some embodiments, the human α-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 34-549 of SEQ ID NO:1. In some embodiments, the human α-Klotho polypeptide comprises an amino acid sequence of amino acids 34-549 of SEQ ID NO:1.

In some embodiments, the human α-Klotho polypeptide comprises an amino acid sequence having at least 95% identity or at least 99% identity to amino acids 34-506 of SEQ ID NO:1 (the full-length, wild-type sequence of the human Klotho precursor protein—NP004786). In some embodiments, the human α-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 34-506 of SEQ ID NO:1. In some embodiments, the human α-Klotho polypeptide comprises an amino acid sequence of amino acids 34-506 of SEQ ID NO:1.

In some embodiments, the α-Klotho polypeptide is a recombinant α-Klotho polypeptide. In some such embodiments, the recombinant Klotho polypeptide is modified with a water-soluble polypeptide. For example, in some embodiments, the recombinant Klotho polypeptide is chemically or enzymatically modified in-vitro. In some embodiments, the recombinant Klotho polypeptide is modified with, e.g., polyethylene glycol (PEG), polysialic acid, and/or hydroxyethyl starch.

In some embodiments, the recombinant α-Klotho polypeptide is a fusion protein with a half-life extending peptide moiety (e.g., an Fc domain, albumin polypeptide, albumin-binding peptide, and/or XTEN peptide).

In some embodiments, the α-Klotho polypeptide is purified from a pool of blood plasma or blood serum from at least 1000 donors. In some embodiments, the α-Klotho polypeptide is purified from blood plasma or blood serum from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors. In some embodiments, the α-Klotho polypeptide is purified from a pool of tissue samples obtained from at least 1000 donors. In some embodiments, the α-Klotho polypeptide is purified from a tissue sample from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors.

In some embodiments, the α-Klotho polypeptide is administered by intravenous infusion. In some embodiments, the α-Klotho polypeptide is administered by subcutaneous injection. In some embodiments, the α-Klotho polypeptide is administered to the subject by any suitable means to treat the disease or disorder. For example, in certain embodiments, the α-Klotho polypeptide is administered by intravenous, intraocular, subcutaneous, and/or intramuscular means. In some embodiments, the α-Klotho polypeptide is administered by parenteral (including intravenous, intradermal, intraperitoneal, intramuscular and subcutaneous) routes or by other delivery routes, including oral, nasal, buccal, sublingual, intra-tracheal, transdermal, transmucosal, and pulmonary. In some embodiments, the α-Klotho polypeptide is administered either systemically or locally (e.g., directly). Systemic administration includes: oral, transdermal, subdermal, intraperitioneal, subcutaneous, transnasal, sublingual, or rectal. In some embodiments, the α-Klotho polypeptide is administered via a sustained delivery device implanted, for example, subcutaneously or intramuscularly. In some embodiments, the α-Klotho polypeptide is administered by continuous release or delivery, using, for example, an infusion pump, continuous infusion, controlled release formulations utilizing polymer, oil or water insoluble matrices.

In some embodiments, the α-Klotho polypeptide is administered to a subject alone or in combination with other compositions. In some embodiments, the α-Klotho polypeptide is administered at periodic intervals, over multiple time points, and/or for a duration of treatment. For example, in some such embodiments, the α-Klotho polypeptide is administered at least every 1, 2, 3, 4, 6, 8, 12, or 24 hours, at least every 1, 2, 3, 4, 5, 6, or 7 days, at least every 1, 2, 3 or 4 weeks, or at least at a monthly, bi-monthly, annually or bi-annually frequency. In some embodiments, the α-Klotho polypeptide is administered at a single time point. In some embodiments, the time needed to complete a course of the treatment is determined by a physician. In some embodiments, the course of treatment ranges from as short as one day to more than a month. In certain embodiments, a course of treatment can be from 1 to 6 months, or more than 6 months.

In some embodiments, the α-Klotho polypeptide is administered in extended release form, which is capable of releasing the protein over a predetermined release period, such that a therapeutically effective plasma level of the polypeptide is maintained for at least 24 hours, such as at least 48 hours, at least 72 hours, at least one week, or at least one month.

In some embodiments, the α-Klotho polypeptide is administered in a formulation that is selected for the mode of delivery, e.g., intravenous, intraocular, subcutaneous, and/or intramuscular means. In some embodiments, the α-Klotho polypeptide is administered in combination with one or more active therapeutic agents for treating co-infections or associated complications.

Another aspect of the present disclosure provides a method for treating a severe acute respiratory syndrome-related coronavirus (SARS-CoV) infection in a subject in need thereof. The method comprises determining whether the subject has diminished Klotho activity by obtaining a blood sample from the subject, determining an amount of Klotho protein in the blood sample or a level of Klotho activity in the blood sample, and comparing the amount of Klotho protein in the blood sample or the level of Klotho activity in the blood sample to a predetermined threshold, thus determining whether the subject has diminished Klotho activity. When the subject has diminished Klotho activity, a first therapy for SARS-CoV infection is administered to the subject; and when the subject does not have diminished Klotho activity, a second therapy for SARS-CoV infection is administered to the subject that is different from the first therapy.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the coronavirus infection is a Middle East respiratory syndrome coronavirus (MERS-CoV) infection. In some embodiments, the subject has been diagnosed with MERS or camel flu.

In some embodiments, the Klotho protein is α-Klotho. In some embodiments, the Klotho protein is β-Klotho. In some embodiments, the Klotho protein is γ-Klotho. In some embodiments, the amount of Klotho protein in the blood sample or the level of Klotho activity in the blood sample that is determined is based on an amount and/or an activity of α-Klotho, β-Klotho, or γ-Klotho.

In some embodiments, the first therapy comprises administering a therapeutically effective amount of a Klotho polypeptide to the subject. In some embodiments, the therapeutically effective amount of a Klotho polypeptide to the subject is a therapeutically effective amount of α-Klotho polypeptide. In some embodiments, the first treatment is more aggressive than the second treatment.

Beta-Klotho Polypeptide Treatment for Coronavirus Infection

One aspect of the present disclosure provides a method for treating a severe acute respiratory syndrome-related coronavirus (SARS-CoV) infection in a subject in need thereof, the method comprising administering a therapeutically effective amount of a Klotho polypeptide to the subject.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the present disclosure provides a method for treating a coronavirus infection, where the coronavirus infection is a Middle East respiratory syndrome coronavirus (MERS-CoV) infection. In some embodiments, the subject has been diagnosed with MERS or camel flu.

In some embodiments, the Klotho polypeptide is a β-Klotho polypeptide. In some embodiments, the β-Klotho polypeptide is any of the embodiments described herein (e.g., see Definitions: Klotho polypeptide). For example, in some embodiments, the β-Klotho polypeptide comprises a KL1 glycosyl hydrolase-1 domain and a KL2 glycosyl hydrolase-2 domain. In some alternative embodiments, the β-Klotho polypeptide comprises a KL1 glycosyl hydrolase-1 domain, but not a KL2 glycosyl hydrolase-2 domain.

In some embodiments, the β-Klotho polypeptide is a human β-Klotho polypeptide. In some embodiments, the human β-Klotho polypeptide comprises an amino acid sequence having at least 95% identity or at least 99% identity to amino acids 54-996 of SEQ ID NO:2 (the full-length, wild-type sequence of the human β-Klotho precursor protein—NP783864). In some embodiments, the human β-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 54-996 of SEQ ID NO:2. In some embodiments, the human β-Klotho polypeptide comprises an amino acid sequence of amino acids 54-996 of SEQ ID NO:2.

In some embodiments, the human β-Klotho polypeptide comprises an amino acid sequence having at least 95% identity or at least 99% identity to amino acids 77-508 of SEQ ID NO:2 (the full-length, wild-type sequence of the human β-Klotho precursor protein—NP783864). In some embodiments, the human β-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 77-508 of SEQ ID NO:2. In some embodiments, the human β-Klotho polypeptide comprises an amino acid sequence of amino acids 77-508 of SEQ ID NO:2.

In some embodiments, the β-Klotho polypeptide is a recombinant β-Klotho polypeptide. In some such embodiments, the recombinant Klotho polypeptide is modified with a water-soluble polypeptide. For example, in some embodiments, the recombinant Klotho polypeptide is chemically or enzymatically modified in-vitro. In some embodiments, the recombinant Klotho polypeptide is modified with, e.g., polyethylene glycol (PEG), polysialic acid, and/or hydroxyethyl starch.

In some embodiments, the recombinant β-Klotho polypeptide is a fusion protein with a half-life extending peptide moiety (e.g., an Fc domain, albumin polypeptide, albumin-binding peptide, and/or XTEN peptide).

In some embodiments, the β-Klotho polypeptide is purified from a pool of blood plasma or blood serum from at least 1000 donors. In some embodiments, the β-Klotho polypeptide is purified from blood plasma or blood serum from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors. In some embodiments, the β-Klotho polypeptide is purified from a pool of tissue samples obtained from at least 1000 donors. In some embodiments, the β-Klotho polypeptide is purified from a tissue sample from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors.

In some embodiments, the β-Klotho polypeptide is administered by intravenous infusion. In some embodiments, the β-Klotho polypeptide is administered by subcutaneous injection. In some embodiments, the β-Klotho polypeptide is administered to the subject by any suitable means to treat the disease or disorder. For example, in certain embodiments, the (3-Klotho polypeptide is administered by intravenous, intraocular, subcutaneous, and/or intramuscular means. In some embodiments, the β-Klotho polypeptide is administered by parenteral (including intravenous, intradermal, intraperitoneal, intramuscular and subcutaneous) routes or by other delivery routes, including oral, nasal, buccal, sublingual, intra-tracheal, transdermal, transmucosal, and pulmonary. In some embodiments, the β-Klotho polypeptide is administered either systemically or locally (e.g., directly). Systemic administration includes: oral, transdermal, subdermal, intraperitioneal, subcutaneous, transnasal, sublingual, or rectal. In some embodiments, the β-Klotho polypeptide is administered via a sustained delivery device implanted, for example, subcutaneously or intramuscularly. In some embodiments, the β-Klotho polypeptide is administered by continuous release or delivery, using, for example, an infusion pump, continuous infusion, controlled release formulations utilizing polymer, oil or water insoluble matrices.

In some embodiments, the β-Klotho polypeptide is administered to a subject alone or in combination with other compositions. In some embodiments, the β-Klotho polypeptide is administered at periodic intervals, over multiple time points, and/or for a duration of treatment. For example, in some such embodiments, the β-Klotho polypeptide is administered at least every 1, 2, 3, 4, 6, 8, 12, or 24 hours, at least every 1, 2, 3, 4, 5, 6, or 7 days, at least every 1, 2, 3 or 4 weeks, or at least at a monthly, bi-monthly, annually or bi-annually frequency. In some embodiments, the β-Klotho polypeptide is administered at a single time point. In some embodiments, the time needed to complete a course of the treatment is determined by a physician. In some embodiments, the course of treatment ranges from as short as one day to more than a month. In certain embodiments, a course of treatment can be from 1 to 6 months, or more than 6 months.

In some embodiments, the β-Klotho polypeptide is administered in extended release form, which is capable of releasing the protein over a predetermined release period, such that a therapeutically effective plasma level of the polypeptide is maintained for at least 24 hours, such as at least 48 hours, at least 72 hours, at least one week, or at least one month.

In some embodiments, the β-Klotho polypeptide is administered in a formulation that is selected for the mode of delivery, e.g., intravenous, intraocular, subcutaneous, and/or intramuscular means. In some embodiments, the β-Klotho polypeptide is administered in combination with one or more active therapeutic agents for treating co-infections or associated complications.

Another aspect of the present disclosure provides a method for treating a severe acute respiratory syndrome-related coronavirus (SARS-CoV) infection in a subject in need thereof. The method comprises determining whether the subject has diminished Klotho activity by obtaining a blood sample from the subject, determining an amount of Klotho protein in the blood sample or a level of Klotho activity in the blood sample, and comparing the amount of Klotho protein in the blood sample or the level of Klotho activity in the blood sample to a predetermined threshold, thus determining whether the subject has diminished Klotho activity. When the subject has diminished Klotho activity, a first therapy for SARS-CoV infection is administered to the subject; and when the subject does not have diminished Klotho activity, a second therapy for SARS-CoV infection is administered to the subject that is different from the first therapy.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the coronavirus infection is a Middle East respiratory syndrome coronavirus (MERS-CoV) infection. In some embodiments, the subject has been diagnosed with MERS or camel flu.

In some embodiments, the Klotho protein is α-Klotho. In some embodiments, the Klotho protein is β-Klotho. In some embodiments, the Klotho protein is γ-Klotho. In some embodiments, the amount of Klotho protein in the blood sample or the level of Klotho activity in the blood sample that is determined is based on an amount and/or an activity of α-Klotho, β-Klotho, or γ-Klotho.

In some embodiments, the first therapy comprises administering a therapeutically effective amount of a Klotho polypeptide to the subject. In some embodiments, the therapeutically effective amount of a Klotho polypeptide to the subject is a therapeutically effective amount of β-Klotho polypeptide. In some embodiments, the first treatment is more aggressive than the second treatment.

Gamma-Klotho Polypeptide Treatment for Coronavirus Infection

One aspect of the present disclosure provides a method for treating a severe acute respiratory syndrome-related coronavirus (SARS-CoV) infection in a subject in need thereof, the method comprising administering a therapeutically effective amount of a Klotho polypeptide to the subject.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the present disclosure provides a method for treating a coronavirus infection, where the coronavirus infection is a Middle East respiratory syndrome coronavirus (MERS-CoV) infection. In some embodiments, the subject has been diagnosed with MERS or camel flu.

In some embodiments, the Klotho polypeptide is a γ-Klotho polypeptide. In some embodiments, the γ-Klotho polypeptide is any of the embodiments described herein (e.g., see Definitions: Klotho polypeptide). For example, in some embodiments, the γ-Klotho polypeptide is a human γ-Klotho polypeptide.

In some embodiments, the human γ-Klotho polypeptide comprises an amino acid sequence having at least 95% identity or at least 99% identity to amino acids 23-541 of SEQ ID NO:3 (the full-length, wild-type sequence of the human γ-Klotho precursor protein—NP_997221). In some embodiments, the human γ-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 23-541 of SEQ ID NO:3. In some embodiments, the human γ-Klotho polypeptide comprises an amino acid sequence of amino acids 23-541 of SEQ ID NO:3.

In some embodiments, the γ-Klotho polypeptide is a recombinant γ-Klotho polypeptide. In some such embodiments, the recombinant Klotho polypeptide is modified with a water-soluble polypeptide. For example, in some embodiments, the recombinant Klotho polypeptide is chemically or enzymatically modified in-vitro. In some embodiments, the recombinant Klotho polypeptide is modified with, e.g., polyethylene glycol (PEG), polysialic acid, and/or hydroxyethyl starch.

In some embodiments, the recombinant γ-Klotho polypeptide is a fusion protein with a half-life extending peptide moiety (e.g., an Fc domain, albumin polypeptide, albumin-binding peptide, and/or XTEN peptide).

In some embodiments, the γ-Klotho polypeptide is purified from a pool of blood plasma or blood serum from at least 1000 donors. In some embodiments, the γ-Klotho polypeptide is purified from blood plasma or blood serum from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors. In some embodiments, the γ-Klotho polypeptide is purified from a pool of tissue samples obtained from at least 1000 donors. In some embodiments, the γ-Klotho polypeptide is purified from a tissue sample from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors.

In some embodiments, the γ-Klotho polypeptide is administered by intravenous infusion. In some embodiments, the γ-Klotho polypeptide is administered by subcutaneous injection. In some embodiments, the γ-Klotho polypeptide is administered to the subject by any suitable means to treat the disease or disorder. For example, in certain embodiments, the γ-Klotho polypeptide is administered by intravenous, intraocular, subcutaneous, and/or intramuscular means. In some embodiments, the γ-Klotho polypeptide is administered by parenteral (including intravenous, intradermal, intraperitoneal, intramuscular and subcutaneous) routes or by other delivery routes, including oral, nasal, buccal, sublingual, intra-tracheal, transdermal, transmucosal, and pulmonary. In some embodiments, the γ-Klotho polypeptide is administered either systemically or locally (e.g., directly). Systemic administration includes: oral, transdermal, subdermal, intraperitioneal, subcutaneous, transnasal, sublingual, or rectal. In some embodiments, the γ-Klotho polypeptide is administered via a sustained delivery device implanted, for example, subcutaneously or intramuscularly. In some embodiments, the γ-Klotho polypeptide is administered by continuous release or delivery, using, for example, an infusion pump, continuous infusion, controlled release formulations utilizing polymer, oil or water insoluble matrices.

In some embodiments, the γ-Klotho polypeptide is administered to a subject alone or in combination with other compositions. In some embodiments, the γ-Klotho polypeptide is administered at periodic intervals, over multiple time points, and/or for a duration of treatment. For example, in some such embodiments, the γ-Klotho polypeptide is administered at least every 1, 2, 3, 4, 6, 8, 12, or 24 hours, at least every 1, 2, 3, 4, 5, 6, or 7 days, at least every 1, 2, 3 or 4 weeks, or at least at a monthly, bi-monthly, annually or bi-annually frequency. In some embodiments, the γ-Klotho polypeptide is administered at a single time point. In some embodiments, the time needed to complete a course of the treatment is determined by a physician. In some embodiments, the course of treatment ranges from as short as one day to more than a month. In certain embodiments, a course of treatment can be from 1 to 6 months, or more than 6 months.

In some embodiments, the γ-Klotho polypeptide is administered in extended release form, which is capable of releasing the protein over a predetermined release period, such that a therapeutically effective plasma level of the polypeptide is maintained for at least 24 hours, such as at least 48 hours, at least 72 hours, at least one week, or at least one month.

In some embodiments, the γ-Klotho polypeptide is administered in a formulation that is selected for the mode of delivery, e.g., intravenous, intraocular, subcutaneous, and/or intramuscular means. In some embodiments, the γ-Klotho polypeptide is administered in combination with one or more active therapeutic agents for treating co-infections or associated complications.

Another aspect of the present disclosure provides a method for treating a severe acute respiratory syndrome-related coronavirus (SARS-CoV) infection in a subject in need thereof. The method comprises determining whether the subject has diminished Klotho activity by obtaining a blood sample from the subject, determining an amount of Klotho protein in the blood sample or a level of Klotho activity in the blood sample, and comparing the amount of Klotho protein in the blood sample or the level of Klotho activity in the blood sample to a predetermined threshold, thus determining whether the subject has diminished Klotho activity. When the subject has diminished Klotho activity, a first therapy for SARS-CoV infection is administered to the subject; and when the subject does not have diminished Klotho activity, a second therapy for SARS-CoV infection is administered to the subject that is different from the first therapy.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the coronavirus infection is a Middle East respiratory syndrome coronavirus (MERS-CoV) infection. In some embodiments, the subject has been diagnosed with MERS or camel flu.

In some embodiments, the Klotho protein is α-Klotho. In some embodiments, the Klotho protein is β-Klotho. In some embodiments, the Klotho protein is γ-Klotho. In some embodiments, the amount of Klotho protein in the blood sample or the level of Klotho activity in the blood sample that is determined is based on an amount and/or an activity of α-Klotho, β-Klotho, or γ-Klotho.

In some embodiments, the first therapy comprises administering a therapeutically effective amount of a Klotho polypeptide to the subject. In some embodiments, the therapeutically effective amount of a Klotho polypeptide to the subject is a therapeutically effective amount of γ-Klotho polypeptide. In some embodiments, the first treatment is more aggressive than the second treatment.

### Klotho Gene Therapy for Coronavirus Infection

Alpha-Klotho Gene Therapy for Coronavirus Infection

Another aspect of the present disclosure provides a method for treating a severe acute respiratory syndrome-related coronavirus (SARS-CoV) infection in a subject in need thereof, the method comprising administering a Klotho polynucleotide encoding a Klotho polypeptide to the subject.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the present disclosure provides a method for treating a coronavirus infection, where the coronavirus infection is a Middle East respiratory syndrome coronavirus (MERS-CoV) infection. In some embodiments, the subject has been diagnosed with MERS or camel flu.

In some embodiments, the Klotho polynucleotide encodes an α-Klotho polypeptide (e.g., an α-Klotho polynucleotide). In some embodiments, the α-Klotho polypeptide encoded by the α-Klotho polynucleotide is any of the embodiments described herein (e.g., see Definitions: Klotho polypeptide). For example, in some embodiments, the α-Klotho polypeptide comprises a KL1 glycosyl hydrolase-1 domain and a KL2 glycosyl hydrolase-2 domain. In some alternative embodiments, the α-Klotho polypeptide comprises a KL1 glycosyl hydrolase-1 domain, but not a KL2 glycosyl hydrolase-2 domain.

In some embodiments, the α-Klotho polypeptide is a human α-Klotho polypeptide. In some embodiments, the human α-Klotho polypeptide comprises an amino acid sequence having at least 95% identity or at least 99% identity to amino acids 34-981 of SEQ ID NO:1 (the full-length, wild-type sequence of the human Klotho precursor protein—NP004786). In some embodiments, the human α-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 34-981 of SEQ ID NO:1. In some embodiments, the human α-Klotho polypeptide comprises an amino acid sequence of amino acids 34-981 of SEQ ID NO:1.

In some embodiments, the human α-Klotho polypeptide comprises an amino acid sequence having at least 95% identity or at least 99% identity to amino acids 34-549 of SEQ ID NO:1 (the full-length, wild-type sequence of the human Klotho precursor protein—NP004786). In some embodiments, the human α-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 34-549 of SEQ ID NO:1. In some embodiments, the human α-Klotho polypeptide comprises an amino acid sequence of amino acids 34-549 of SEQ ID NO:1.

In some embodiments, the human α-Klotho polypeptide comprises an amino acid sequence having at least 95% identity or at least 99% identity to amino acids 34-506 of SEQ ID NO:1 (the full-length, wild-type sequence of the human Klotho precursor protein—NP004786). In some embodiments, the human α-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 34-506 of SEQ ID NO:1. In some embodiments, the human α-Klotho polypeptide comprises an amino acid sequence of amino acids 34-506 of SEQ ID NO:1.

One skilled in the art will perceive, based on the amino acid sequence of the Klotho polypeptide and/or any variants disclosed above, a respective nucleic acid sequence coding for any such amino acid sequence based on the genetic code.

In some embodiments, the α-Klotho polynucleotide encodes a recombinant Klotho polypeptide. In some such embodiments, the α-Klotho polynucleotide encodes a Klotho polypeptide that is modified with a water-soluble polypeptide. In some embodiments, the Klotho polynucleotide encodes a Klotho polypeptide that is chemically or enzymatically modified in-vitro. In some embodiments, the Klotho polynucleotide encodes a Klotho polypeptide that is modified with, e.g., polyethylene glycol (PEG), polysialic acid, and/or hydroxyethyl starch.

In some embodiments, the α-Klotho polynucleotide encodes a recombinant α-Klotho polypeptide that is a fusion protein with a half-life extending peptide moiety (e.g., an Fc domain, albumin polypeptide, albumin-binding peptide, and/or XTEN peptide).

In some embodiments, the α-Klotho polynucleotide is purified (e.g., isolated and/or amplified) from a pool of blood plasma or blood serum from at least 1000 donors. In some embodiments, the α-Klotho polynucleotide is purified from blood plasma or blood serum from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors. In some embodiments, the α-Klotho polynucleotide is purified from a pool of tissue samples obtained from at least 1000 donors. In some embodiments, the α-Klotho polynucleotide is purified from a tissue sample from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors.

In some embodiments, the α-Klotho polynucleotide sequence is obtained from a sequencing of nucleic acids obtained from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 donors.

In some embodiments, the α-Klotho polynucleotide is administered by intravenous infusion. In some embodiments, the α-Klotho polynucleotide is administered by subcutaneous injection. In some embodiments, the α-Klotho polynucleotide is administered to the subject by any suitable means to treat the disease or disorder. For example, in certain embodiments, the α-Klotho polynucleotide is administered by intravenous, intraocular, subcutaneous, and/or intramuscular means. In some embodiments, the α-Klotho polynucleotide is administered by parenteral (including intravenous, intradermal, intraperitoneal, intramuscular and subcutaneous) routes or by other delivery routes, including oral, nasal, buccal, sublingual, intra-tracheal, transdermal, transmucosal, and pulmonary. In some embodiments, the α-Klotho polynucleotide is administered either systemically or locally (e.g., directly). Systemic administration includes: oral, transdermal, subdermal, intraperitioneal, subcutaneous, transnasal, sublingual, or rectal. In some embodiments, the α-Klotho polynucleotide is administered via a sustained delivery device implanted, for example, subcutaneously or intramuscularly. In some embodiments, the α-Klotho polynucleotide is administered by continuous release or delivery, using, for example, an infusion pump, continuous infusion, controlled release formulations utilizing polymer, oil or water insoluble matrices.

In some embodiments, the α-Klotho polynucleotide is administered to a subject alone or in combination with other compositions. In some embodiments, the α-Klotho polynucleotide is administered at periodic intervals, over multiple time points, and/or for a duration of treatment. For example, in some such embodiments, the α-Klotho polynucleotide is administered at least every 1, 2, 3, 4, 6, 8, 12, or 24 hours, at least every 1, 2, 3, 4, 5, 6, or 7 days, at least every 1, 2, 3 or 4 weeks, or at least at a monthly, bi-monthly, annually or bi-annually frequency. In some embodiments, the α-Klotho polynucleotide is administered at a single time point. In some embodiments, the time needed to complete a course of the treatment is determined by a physician. In some embodiments, the course of treatment ranges from as short as one day to more than a month. In certain embodiments, a course of treatment can be from 1 to 6 months, or more than 6 months.

In some embodiments, the α-Klotho polynucleotide is administered in extended release form, which is capable of releasing the protein over a predetermined release period, such that a therapeutically effective plasma level of the polynucleotide is maintained for at least 24 hours, such as at least 48 hours, at least 72 hours, at least one week, or at least one month.

In some embodiments, the α-Klotho polynucleotide is administered in a formulation that is selected for the mode of delivery, e.g., intravenous, intraocular, subcutaneous, and/or intramuscular means. In some embodiments, the α-Klotho polynucleotide is administered in combination with one or more active therapeutic agents for treating co-infections or associated complications.

In some embodiments, the method comprises administering to the subject a viral-based gene therapy vector comprising the α-Klotho polynucleotide. In some embodiments, the viral-based gene therapy vector is an adeno-associated viral (AAV) gene therapy vector.

In some embodiments, a therapeutically effective amount of a α-Klotho polynucleotide comprises, for example, a construct comprising the therapeutic agent (e.g., the α-Klotho polynucleotide), a vector comprising the therapeutic agent (e.g., the α-Klotho polynucleotide), a plasmid comprising the therapeutic agent (e.g., the α-Klotho polynucleotide), and/or a host cell comprising the therapeutic agent (e.g., the α-Klotho polynucleotide). In some embodiments, the gene therapy comprises a recombinant vector suitable for gene therapy (e.g., an adeno-associated virus, adenovirus, nanoparticle, plasmid, and/or lentivirus).

Another aspect of the present disclosure provides a method for treating a severe acute respiratory syndrome-related coronavirus (SARS-CoV) infection in a subject in need thereof. The method comprising determining whether the subject has diminished Klotho activity by obtaining a blood sample from the subject, determining an amount of Klotho protein in the blood sample or a level of Klotho activity in the blood sample, and comparing the amount of Klotho protein in the blood sample or the level of Klotho activity in the blood sample to a predetermined threshold, thus determining whether the subject has diminished Klotho activity. When the subject has diminished Klotho activity, a first therapy for SARS-CoV infection is administered to the subject; and when the subject does not have diminished Klotho activity, a second therapy for SARS-CoV infection is administered to the subject that is different from the first therapy.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the coronavirus infection is a Middle East respiratory syndrome coronavirus (MERS-CoV) infection. In some embodiments, the subject has been diagnosed with MERS or camel flu.

In some embodiments, the Klotho protein is α-Klotho. In some embodiments, the Klotho protein is β-Klotho. In some embodiments, the Klotho protein is γ-Klotho. In some embodiments, the amount of Klotho protein in the blood sample or the level of Klotho activity in the blood sample that is determined is based on an amount and/or an activity of α-Klotho, β-Klotho, or γ-Klotho.

In some embodiments, the first therapy comprises administering α-Klotho polynucleotide encoding α-Klotho polypeptide to the subject. In some embodiments, the first therapy further comprises administering to the subject a viral-based gene therapy vector comprising the α-Klotho polynucleotide. In some such embodiments, the viral-based gene therapy vector is an adeno-associated viral (AAV) gene therapy vector. In some embodiments, the first treatment is more aggressive than the second treatment.

Beta-Klotho Gene Therapy for Coronavirus Infection

Another aspect of the present disclosure provides a method for treating a severe acute respiratory syndrome-related coronavirus (SARS-CoV) infection in a subject in need thereof, the method comprising administering a Klotho polynucleotide encoding a Klotho polypeptide to the subject.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the present disclosure provides a method for treating a coronavirus infection, where the coronavirus infection is a Middle East respiratory syndrome coronavirus (MERS-CoV) infection. In some embodiments, the subject has been diagnosed with MERS or camel flu.

In some embodiments, the Klotho polynucleotide encodes a β-Klotho polypeptide (e.g., a β-Klotho polynucleotide). In some embodiments, the β-Klotho polypeptide encoded by the 3-Klotho polynucleotide is any of the embodiments described herein (e.g., see Definitions: Klotho polypeptide). For example, in some embodiments, the β-Klotho polypeptide comprises a KL1 glycosyl hydrolase-1 domain and a KL2 glycosyl hydrolase-2 domain. In some alternative embodiments, the β-Klotho polypeptide comprises a KL1 glycosyl hydrolase-1 domain, but not a KL2 glycosyl hydrolase-2 domain.

In some embodiments, the β-Klotho polypeptide is a human β-Klotho polypeptide. In some embodiments, the human β-Klotho polypeptide comprises an amino acid sequence having at least 95% identity or at least 99% identity to amino acids 54-996 of SEQ ID NO:2 (the full-length, wild-type sequence of the human β-Klotho precursor protein—NP783864). In some embodiments, the human β-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 54-996 of SEQ ID NO:2. In some embodiments, the human β-Klotho polypeptide comprises an amino acid sequence of amino acids 54-996 of SEQ ID NO:2.

In some embodiments, the human β-Klotho polypeptide comprises an amino acid sequence having at least 95% identity or at least 99% identity to amino acids 77-508 of SEQ ID NO:2 (the full-length, wild-type sequence of the human β-Klotho precursor protein—NP783864). In some embodiments, the human β-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 77-508 of SEQ ID NO:2. In some embodiments, the human β-Klotho polypeptide comprises an amino acid sequence of amino acids 77-508 of SEQ ID NO:2.

One skilled in the art will perceive, based on the amino acid sequence of the Klotho polypeptide and/or any variants disclosed above, a respective nucleic acid sequence coding for any such amino acid sequence based on the genetic code.

In some embodiments, the β-Klotho polynucleotide encodes a recombinant β-Klotho polypeptide. In some such embodiments, the β-Klotho polynucleotide encodes a Klotho polypeptide that is modified with a water-soluble polypeptide. In some embodiments, the Klotho polynucleotide encodes a Klotho polypeptide that is chemically or enzymatically modified in-vitro. In some embodiments, the β-Klotho polynucleotide encodes a Klotho polypeptide that is modified with, e.g., polyethylene glycol (PEG), polysialic acid, and/or hydroxyethyl starch.

In some embodiments, the β-Klotho polynucleotide encodes a recombinant β-Klotho polypeptide that is a fusion protein with a half-life extending peptide moiety (e.g., an Fc domain, albumin polypeptide, albumin-binding peptide, and/or XTEN peptide).

In some embodiments, the β-Klotho polynucleotide is purified (e.g., isolated and/or amplified) from a pool of blood plasma or blood serum from at least 1000 donors. In some embodiments, the β-Klotho polynucleotide is purified from blood plasma or blood serum from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors. In some embodiments, the β-Klotho polynucleotide is purified from a pool of tissue samples obtained from at least 1000 donors. In some embodiments, the β-Klotho polynucleotide is purified from a tissue sample from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors.

In some embodiments, the β-Klotho polynucleotide sequence is obtained from a sequencing of nucleic acids obtained from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 donors.

In some embodiments, the β-Klotho polynucleotide is administered by intravenous infusion. In some embodiments, the β-Klotho polynucleotide is administered by subcutaneous injection. In some embodiments, the β-Klotho polynucleotide is administered to the subject by any suitable means to treat the disease or disorder. For example, in certain embodiments, the β-Klotho polynucleotide is administered by intravenous, intraocular, subcutaneous, and/or intramuscular means. In some embodiments, the β-Klotho polynucleotide is administered by parenteral (including intravenous, intradermal, intraperitoneal, intramuscular and subcutaneous) routes or by other delivery routes, including oral, nasal, buccal, sublingual, intra-tracheal, transdermal, transmucosal, and pulmonary. In some embodiments, the β-Klotho polynucleotide is administered either systemically or locally (e.g., directly). Systemic administration includes: oral, transdermal, subdermal, intraperitioneal, subcutaneous, transnasal, sublingual, or rectal. In some embodiments, the β-Klotho polynucleotide is administered via a sustained delivery device implanted, for example, subcutaneously or intramuscularly. In some embodiments, the β-Klotho polynucleotide is administered by continuous release or delivery, using, for example, an infusion pump, continuous infusion, controlled release formulations utilizing polymer, oil or water insoluble matrices.

In some embodiments, the β-Klotho polynucleotide is administered to a subject alone or in combination with other compositions. In some embodiments, the β-Klotho polynucleotide is administered at periodic intervals, over multiple time points, and/or for a duration of treatment. For example, in some such embodiments, the β-Klotho polynucleotide is administered at least every 1, 2, 3, 4, 6, 8, 12, or 24 hours, at least every 1, 2, 3, 4, 5, 6, or 7 days, at least every 1, 2, 3 or 4 weeks, or at least at a monthly, bi-monthly, annually or bi-annually frequency. In some embodiments, the β-Klotho polynucleotide is administered at a single time point. In some embodiments, the time needed to complete a course of the treatment is determined by a physician. In some embodiments, the course of treatment ranges from as short as one day to more than a month. In certain embodiments, a course of treatment can be from 1 to 6 months, or more than 6 months.

In some embodiments, the β-Klotho polynucleotide is administered in extended release form, which is capable of releasing the protein over a predetermined release period, such that a therapeutically effective plasma level of the polynucleotide is maintained for at least 24 hours, such as at least 48 hours, at least 72 hours, at least one week, or at least one month.

In some embodiments, the β-Klotho polynucleotide is administered in a formulation that is selected for the mode of delivery, e.g., intravenous, intraocular, subcutaneous, and/or intramuscular means. In some embodiments, the β-Klotho polynucleotide is administered in combination with one or more active therapeutic agents for treating co-infections or associated complications.

In some embodiments, the method comprises administering to the subject a viral-based gene therapy vector comprising the β-Klotho polynucleotide. In some embodiments, the viral-based gene therapy vector is an adeno-associated viral (AAV) gene therapy vector.

In some embodiments, a therapeutically effective amount of a β-Klotho polynucleotide comprises, for example, a construct comprising the therapeutic agent (e.g., the β-Klotho polynucleotide), a vector comprising the therapeutic agent (e.g., the β-Klotho polynucleotide), a plasmid comprising the therapeutic agent (e.g., the β-Klotho polynucleotide), and/or a host cell comprising the therapeutic agent (e.g., the β-Klotho polynucleotide). In some embodiments, the gene therapy comprises a recombinant vector suitable for gene therapy (e.g., an adeno-associated virus, adenovirus, nanoparticle, plasmid, and/or lentivirus).

Another aspect of the present disclosure provides a method for treating a severe acute respiratory syndrome-related coronavirus (SARS-CoV) infection in a subject in need thereof. The method comprising determining whether the subject has diminished Klotho activity by obtaining a blood sample from the subject, determining an amount of Klotho protein in the blood sample or a level of Klotho activity in the blood sample, and comparing the amount of Klotho protein in the blood sample or the level of Klotho activity in the blood sample to a predetermined threshold, thus determining whether the subject has diminished Klotho activity. When the subject has diminished Klotho activity, a first therapy for SARS-CoV infection is administered to the subject; and when the subject does not have diminished Klotho activity, a second therapy for SARS-CoV infection is administered to the subject that is different from the first therapy.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the coronavirus infection is a Middle East respiratory syndrome coronavirus (MERS-CoV) infection. In some embodiments, the subject has been diagnosed with MERS or camel flu.

In some embodiments, the Klotho protein is α-Klotho. In some embodiments, the Klotho protein is β-Klotho. In some embodiments, the Klotho protein is γ-Klotho. In some embodiments, the amount of Klotho protein in the blood sample or the level of Klotho activity in the blood sample that is determined is based on an amount and/or an activity of α-Klotho, β-Klotho, or γ-Klotho.

In some embodiments, the first therapy comprises administering a β-Klotho polynucleotide encoding a β-Klotho polypeptide to the subject. In some embodiments, the first therapy further comprises administering to the subject a viral-based gene therapy vector comprising the β-Klotho polynucleotide. In some such embodiments, the viral-based gene therapy vector is an adeno-associated viral (AAV) gene therapy vector. In some embodiments, the first treatment is more aggressive than the second treatment.

Gamma-Klotho Gene Therapy for Coronavirus Infection

Another aspect of the present disclosure provides a method for treating a severe acute respiratory syndrome-related coronavirus (SARS-CoV) infection in a subject in need thereof, the method comprising administering a Klotho polynucleotide encoding a Klotho polypeptide to the subject.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the present disclosure provides a method for treating a coronavirus infection, where the coronavirus infection is a Middle East respiratory syndrome coronavirus (MERS-CoV) infection. In some embodiments, the subject has been diagnosed with MERS or camel flu.

In some embodiments, the Klotho polynucleotide encodes a γ-Klotho polypeptide (e.g., a γ-Klotho polynucleotide). In some embodiments, the γ-Klotho polypeptide encoded by the γ-Klotho polynucleotide is any of the embodiments described herein (e.g., see Definitions: Klotho polypeptide). For example, in some embodiments, the γ-Klotho polypeptide is a human γ-Klotho polypeptide.

In some embodiments, the human γ-Klotho polypeptide comprises an amino acid sequence having at least 95% identity or at least 99% identity to amino acids 23-541 of SEQ ID NO:3 (the full-length, wild-type sequence of the human γ-Klotho precursor protein—NP_997221). In some embodiments, the human γ-Klotho polypeptide comprises an amino acid sequence having at least 70%, at least 75%, at least 80%, at least 85%, at least 90%, at least 91% at least 92%, at least 93%, at least 94%, at least 95%, at least 96%, at least 97%, at least 98%, or at least 99% identity to amino acids 23-541 of SEQ ID NO:3. In some embodiments, the human γ-Klotho polypeptide comprises an amino acid sequence of amino acids 23-541 of SEQ ID NO:3.

One skilled in the art will perceive, based on the amino acid sequence of the Klotho polypeptide and/or any variants disclosed above, a respective nucleic acid sequence coding for any such amino acid sequence based on the genetic code.

In some embodiments, the γ-Klotho polynucleotide encodes a recombinant γ-Klotho polypeptide. In some such embodiments, the γ-Klotho polynucleotide encodes a Klotho polypeptide that is modified with a water-soluble polypeptide. In some embodiments, the γ-Klotho polynucleotide encodes a Klotho polypeptide that is chemically or enzymatically modified in-vitro. In some embodiments, the γ-Klotho polynucleotide encodes a Klotho polypeptide that is modified with, e.g., polyethylene glycol (PEG), polysialic acid, and/or hydroxyethyl starch.

In some embodiments, the γ-Klotho polynucleotide encodes a recombinant γ-Klotho polypeptide that is a fusion protein with a half-life extending peptide moiety (e.g., an Fc domain, albumin polypeptide, albumin-binding peptide, and/or XTEN peptide).

In some embodiments, the γ-Klotho polynucleotide is purified (e.g., isolated and/or amplified) from a pool of blood plasma or blood serum from at least 1000 donors. In some embodiments, the γ-Klotho polynucleotide is purified from blood plasma or blood serum from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors. In some embodiments, the γ-Klotho polynucleotide is purified from a pool of tissue samples obtained from at least 1000 donors. In some embodiments, the γ-Klotho polynucleotide is purified from a tissue sample from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 donors.

In some embodiments, the γ-Klotho polynucleotide sequence is obtained from a sequencing of nucleic acids obtained from at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 donors.

In some embodiments, the γ-Klotho polynucleotide is administered by intravenous infusion. In some embodiments, the γ-Klotho polynucleotide is administered by subcutaneous injection. In some embodiments, the γ-Klotho polynucleotide is administered to the subject by any suitable means to treat the disease or disorder. For example, in certain embodiments, the γ-Klotho polynucleotide is administered by intravenous, intraocular, subcutaneous, and/or intramuscular means. In some embodiments, the γ-Klotho polynucleotide is administered by parenteral (including intravenous, intradermal, intraperitoneal, intramuscular and subcutaneous) routes or by other delivery routes, including oral, nasal, buccal, sublingual, intra-tracheal, transdermal, transmucosal, and pulmonary. In some embodiments, the γ-Klotho polynucleotide is administered either systemically or locally (e.g., directly). Systemic administration includes: oral, transdermal, subdermal, intraperitioneal, subcutaneous, transnasal, sublingual, or rectal. In some embodiments, the γ-Klotho polynucleotide is administered via a sustained delivery device implanted, for example, subcutaneously or intramuscularly. In some embodiments, the γ-Klotho polynucleotide is administered by continuous release or delivery, using, for example, an infusion pump, continuous infusion, controlled release formulations utilizing polymer, oil or water insoluble matrices.

In some embodiments, the γ-Klotho polynucleotide is administered to a subject alone or in combination with other compositions. In some embodiments, the γ-Klotho polynucleotide is administered at periodic intervals, over multiple time points, and/or for a duration of treatment. For example, in some such embodiments, the γ-Klotho polynucleotide is administered at least every 1, 2, 3, 4, 6, 8, 12, or 24 hours, at least every 1, 2, 3, 4, 5, 6, or 7 days, at least every 1, 2, 3 or 4 weeks, or at least at a monthly, bi-monthly, annually or bi-annually frequency. In some embodiments, the γ-Klotho polynucleotide is administered at a single time point. In some embodiments, the time needed to complete a course of the treatment is determined by a physician. In some embodiments, the course of treatment ranges from as short as one day to more than a month. In certain embodiments, a course of treatment can be from 1 to 6 months, or more than 6 months.

In some embodiments, the γ-Klotho polynucleotide is administered in extended release form, which is capable of releasing the protein over a predetermined release period, such that a therapeutically effective plasma level of the polynucleotide is maintained for at least 24 hours, such as at least 48 hours, at least 72 hours, at least one week, or at least one month.

In some embodiments, the γ-Klotho polynucleotide is administered in a formulation that is selected for the mode of delivery, e.g., intravenous, intraocular, subcutaneous, and/or intramuscular means. In some embodiments, the γ-Klotho polynucleotide is administered in combination with one or more active therapeutic agents for treating co-infections or associated complications.

In some embodiments, the method comprises administering to the subject a viral-based gene therapy vector comprising the γ-Klotho polynucleotide. In some embodiments, the viral-based gene therapy vector is an adeno-associated viral (AAV) gene therapy vector.

In some embodiments, a therapeutically effective amount of a γ-Klotho polynucleotide comprises, for example, a construct comprising the therapeutic agent (e.g., the γ-Klotho polynucleotide), a vector comprising the therapeutic agent (e.g., the γ-Klotho polynucleotide), a plasmid comprising the therapeutic agent (e.g., the γ-Klotho polynucleotide), and/or a host cell comprising the therapeutic agent (e.g., the γ-Klotho polynucleotide). In some embodiments, the gene therapy comprises a recombinant vector suitable for gene therapy (e.g., an adeno-associated virus, adenovirus, nanoparticle, plasmid, and/or lentivirus).

Another aspect of the present disclosure provides a method for treating a severe acute respiratory syndrome-related coronavirus (SARS-CoV) infection in a subject in need thereof. The method comprising determining whether the subject has diminished Klotho activity by obtaining a blood sample from the subject, determining an amount of Klotho protein in the blood sample or a level of Klotho activity in the blood sample, and comparing the amount of Klotho protein in the blood sample or the level of Klotho activity in the blood sample to a predetermined threshold, thus determining whether the subject has diminished Klotho activity. When the subject has diminished Klotho activity, a first therapy for SARS-CoV infection is administered to the subject; and when the subject does not have diminished Klotho activity, a second therapy for SARS-CoV infection is administered to the subject that is different from the first therapy.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the SARS-CoV infection is a severe acute respiratory syndrome-related coronavirus (SARS-CoV-1) infection. In some embodiments, the subject has been diagnosed with SARS.

In some embodiments, the coronavirus infection is a Middle East respiratory syndrome coronavirus (MERS-CoV) infection. In some embodiments, the subject has been diagnosed with MERS or camel flu.

In some embodiments, the Klotho protein is α-Klotho. In some embodiments, the Klotho protein is β-Klotho. In some embodiments, the Klotho protein is γ-Klotho. In some embodiments, the amount of Klotho protein in the blood sample or the level of Klotho activity in the blood sample that is determined is based on an amount and/or an activity of α-Klotho, β-Klotho, or γ-Klotho.

In some embodiments, the first therapy comprises administering a γ-Klotho polynucleotide encoding a γ-Klotho polypeptide to the subject. In some embodiments, the first therapy further comprises administering to the subject a viral-based gene therapy vector comprising the γ-Klotho polynucleotide. In some such embodiments, the viral-based gene therapy vector is an adeno-associated viral (AAV) gene therapy vector. In some embodiments, the first treatment is more aggressive than the second treatment.

### Therapeutic Compositions

Compositions Comprising Alpha-Klotho

Another aspect of the present disclosure provides a therapeutic composition for the treatment of a severe acute respiratory syndrome-related coronavirus 2 (SARS-CoV-2) infection in a subject in need thereof, comprising a therapeutically effective amount of α-Klotho polypeptide. In some embodiments, the subject has been diagnosed with COVID-19.

Another aspect of the present disclosure provides a therapeutic composition for the treatment of a severe acute respiratory syndrome-related coronavirus (SARS-CoV-1) infection in a subject in need thereof, comprising a therapeutically effective amount of α-Klotho polypeptide. In some embodiments, the subject has been diagnosed with SARS.

Another aspect of the present disclosure provides a therapeutic composition for the treatment of a Middle East respiratory syndrome coronavirus (MERS-CoV) infection in a subject in need thereof, comprising a therapeutically effective amount of α-Klotho polypeptide. In some embodiments, the subject has been diagnosed with MERS or camel flu.

In some embodiments, the therapeutic composition comprises a formulation that includes carriers, stabilizers, diluents, adjuvents and/or other excipients. Carriers or excipients known in the art can also be used to facilitate administration of the polypeptide treatment and/or gene therapy. Examples of carriers and excipients include calcium carbonate, calcium phosphate, various sugars such as lactose, or types of starch, cellulose derivatives, gelatin, vegetable oils, polyethylene glycols and physiologically compatible solvents. Pharmaceutically acceptable carriers include sterile liquids, such as water and oils, including those of petroleum, animal, vegetable or synthetic origin, such as peanut oil, soybean oil, mineral oil, sesame oil and the like. For example, in some embodiments, water is a preferred carrier when the pharmaceutical composition is administered subcutaneously. Saline solutions and aqueous dextrose and glycerol solutions can also be employed as liquid carriers, particularly for injectable solutions.

In some embodiments, the therapeutic composition is thickened with a thickening agent such as methylcellulose. In some embodiments, solutions are prepared in emulsified form, such as either water in oil or oil in water. Any of a wide variety of pharmaceutically acceptable emulsifying agents can be employed including, for example, acacia powder, a non-ionic surfactant (such as a Tween), or an ionic surfactant (such as alkali polyether alcohol sulfates or sulfonates, e.g., a Triton). In general, the composition of the present invention is prepared by mixing the ingredients following generally accepted procedures. For example, the selected components can be simply mixed in a blender or other standard device to produce a concentrated mixture which may then be adjusted to the final concentration and viscosity by the addition of water or thickening agent and possibly a buffer to control pH or an additional solute to control tonicity.

Compositions Comprising Beta-Klotho

Another aspect of the present disclosure provides a therapeutic composition for the treatment of a severe acute respiratory syndrome-related coronavirus 2 (SARS-CoV-2) infection in a subject in need thereof, comprising a therapeutically effective amount of β-Klotho polypeptide. In some embodiments, the subject has been diagnosed with COVID-19.

Another aspect of the present disclosure provides a therapeutic composition for the treatment of a severe acute respiratory syndrome-related coronavirus (SARS-CoV-1) infection in a subject in need thereof, comprising a therapeutically effective amount of β-Klotho polypeptide. In some embodiments, the subject has been diagnosed with SARS.

Another aspect of the present disclosure provides a therapeutic composition for the treatment of a Middle East respiratory syndrome coronavirus (MERS-CoV) infection in a subject in need thereof, comprising a therapeutically effective amount of β-Klotho polypeptide. In some embodiments, the subject has been diagnosed with MERS or camel flu.

In some embodiments, the therapeutic composition comprises a formulation that includes carriers, stabilizers, diluents, adjuvents and/or other excipients. Carriers or excipients known in the art can also be used to facilitate administration of the polypeptide treatment and/or gene therapy. Examples of carriers and excipients include calcium carbonate, calcium phosphate, various sugars such as lactose, or types of starch, cellulose derivatives, gelatin, vegetable oils, polyethylene glycols and physiologically compatible solvents. Pharmaceutically acceptable carriers include sterile liquids, such as water and oils, including those of petroleum, animal, vegetable or synthetic origin, such as peanut oil, soybean oil, mineral oil, sesame oil and the like. For example, in some embodiments, water is a preferred carrier when the pharmaceutical composition is administered subcutaneously. Saline solutions and aqueous dextrose and glycerol solutions can also be employed as liquid carriers, particularly for injectable solutions.

In some embodiments, the therapeutic composition is thickened with a thickening agent such as methylcellulose. In some embodiments, solutions are prepared in emulsified form, such as either water in oil or oil in water. Any of a wide variety of pharmaceutically acceptable emulsifying agents can be employed including, for example, acacia powder, a non-ionic surfactant (such as a Tween), or an ionic surfactant (such as alkali polyether alcohol sulfates or sulfonates, e.g., a Triton). In general, the composition of the present invention is prepared by mixing the ingredients following generally accepted procedures. For example, the selected components can be simply mixed in a blender or other standard device to produce a concentrated mixture which may then be adjusted to the final concentration and viscosity by the addition of water or thickening agent and possibly a buffer to control pH or an additional solute to control tonicity.

Compositions Comprising Gamma-Klotho

Another aspect of the present disclosure provides a therapeutic composition for the treatment of a severe acute respiratory syndrome-related coronavirus 2 (SARS-CoV-2) infection in a subject in need thereof, comprising a therapeutically effective amount of γ-Klotho polypeptide. In some embodiments, the subject has been diagnosed with COVID-19.

Another aspect of the present disclosure provides a therapeutic composition for the treatment of a severe acute respiratory syndrome-related coronavirus (SARS-CoV-1) infection in a subject in need thereof, comprising a therapeutically effective amount of γ-Klotho polypeptide. In some embodiments, the subject has been diagnosed with SARS.

Another aspect of the present disclosure provides a therapeutic composition for the treatment of a Middle East respiratory syndrome coronavirus (MERS-CoV) infection in a subject in need thereof, comprising a therapeutically effective amount of γ-Klotho polypeptide. In some embodiments, the subject has been diagnosed with MERS or camel flu.

In some embodiments, the therapeutic composition comprises a formulation that includes carriers, stabilizers, diluents, adjuvents and/or other excipients. Carriers or excipients known in the art can also be used to facilitate administration of the polypeptide treatment and/or gene therapy. Examples of carriers and excipients include calcium carbonate, calcium phosphate, various sugars such as lactose, or types of starch, cellulose derivatives, gelatin, vegetable oils, polyethylene glycols and physiologically compatible solvents. Pharmaceutically acceptable carriers include sterile liquids, such as water and oils, including those of petroleum, animal, vegetable or synthetic origin, such as peanut oil, soybean oil, mineral oil, sesame oil and the like. For example, in some embodiments, water is a preferred carrier when the pharmaceutical composition is administered subcutaneously. Saline solutions and aqueous dextrose and glycerol solutions can also be employed as liquid carriers, particularly for injectable solutions.

In some embodiments, the therapeutic composition is thickened with a thickening agent such as methylcellulose. In some embodiments, solutions are prepared in emulsified form, such as either water in oil or oil in water. Any of a wide variety of pharmaceutically acceptable emulsifying agents can be employed including, for example, acacia powder, a non-ionic surfactant (such as a Tween), or an ionic surfactant (such as alkali polyether alcohol sulfates or sulfonates, e.g., a Triton). In general, the composition of the present invention is prepared by mixing the ingredients following generally accepted procedures. For example, the selected components can be simply mixed in a blender or other standard device to produce a concentrated mixture which may then be adjusted to the final concentration and viscosity by the addition of water or thickening agent and possibly a buffer to control pH or an additional solute to control tonicity.

### Therapeutic Compounds for Treatment of Coronavirus Infection

Inhibitors of the mTOR Pathway

Klotho is inhibited by the mammalian target of rapamycin (mTOR). As a result, rapamycin indirectly upregulates Klotho, both in vivo and in vitro, by inhibiting mTOR. See, Zhao et al., “Mammalian target of rapamycin signaling inhibition ameliorates vascular calcification via Klotho upregulation,” Kidney Int 88 (2015), which is hereby incorporated by reference herein in its entirety. Whereas mTOR pathways have been shown to play a role in cell injury, oxidative stress, mitochondrial dysfunction, and the onset of hyperinflammation, the use of an mTOR inhibitor improved outcomes for severe H1N1 pneumonia, including hypoxia, multiple organ dysfunction, virus clearance, and shortened recovery times. Such evidence suggests that mTOR and its associated pathways provide potential targets for therapeutic treatment of complications and/or risks associated with COVID-19. See, for example, Wang et al., “Adjuvant Treatment With a Mammalian Target of Rapamycin Inhibitor, Sirolimus, and Steroids Improves Outcomes in Patients With Severe H1N1 Pneumonia and Acute Respiratory Failure,” Crit Care Med 42(2) (2014); and Maiese, “The Mechanistic Target of Rapamycin (mTOR): Novel Considerations as an Antiviral Treatment,” Curr Neur Res 17 (2020), each of which is hereby incorporated by reference herein in its entirety.

The mTOR pathway includes the mechanistic target of rapamycin (mTOR) and its associated pathways of mTOR Complex 1 (mTORC1), mTOR Complex 2 (mTORC2), AMP activated protein (AMPK), phosphoinositide 3-kinase (PI3K) including subunits (e.g., p110α, p110β, p110δ, p110γ, p85α, and p85β), and/or protein kinase B (PKB/AKT). In some embodiments, the mTOR pathway is regulated by PTEN. In some embodiments, activation of the pathway occurs through a receptor tyrosine kinase (e.g., encoded by genes EGFR (ERBB1) and HER2 (ERBB2)). See, for example, Dienstmann et al., “Picking the Point of Inhibition: A Comparative Review of PI3K/AKT/mTOR Pathway Inhibitors,” Mol Cancer Ther 13(5) (2014); and LoRusso, “Inhibition of the PI3K/AKT/mTOR Pathway in Solid Tumors,” J Clin Onc 34(31) (2016), each of which is hereby incorporated by reference herein in its entirety.

Provided herein is a method for treating a severe acute respiratory syndrome-related coronavirus (SARS-CoV) infection in a subject in need thereof, the method comprising administering a therapeutically effective amount of an inhibitor of the mTOR pathway.

In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the inhibitor of the mTOR pathway targets any of the components and/or intermediates of the mTOR pathway. In some such embodiments, the inhibitor of the mTOR pathway induces an upregulation and/or reduces an inhibition of Klotho as a result of the targeting of any of the components and/or intermediates of the mTOR pathway. For example, in some embodiments, the inhibitor of the mTOR pathway targets mTOR, mTOR Complex 1 (mTORC1), mTOR Complex 2 (mTORC2), AMP activated protein (AMPK), phosphoinositide 3-kinase (PI3K) including subunits (e.g., p110α, p110β, p110δ, p¹107, p85α, and p85β), protein kinase B (PKB/AKT), PTEN, and/or receptor tyrosine kinase. In some embodiments, the inhibitor of the mTOR pathway targets phosphoinositide 3-kinase (PI3K). In some embodiments, the phosphoinositide 3-kinase (PI3K) is a Class I PI3K, a Class II PI3K, a Class III PI3K, or a Class IV PI3K. In some embodiments, the catalytic subunit of the Class I PI3K is p110α, p110β, p110δ or p110γ. In some embodiments, the inhibitor is a pan-PI3K class I inhibitor. In some embodiments, the inhibitor is an isoform-specific PI3K inhibitor. In some embodiments, the inhibitor is a dual PI3K/mTOR inhibitor.

In some embodiments, the inhibitor of the mTOR pathway targets protein kinase B (PKB/AKT). In some embodiments, the inhibitor is an AKT inhibitor.

In some embodiments, the inhibitor of the mTOR pathway targets mammalian target of rapamycin (mTOR). In some embodiments, mTOR is a component in mTOR complex 1 (mTORC1) or a component in mTOR complex 2 (mTORC2).

In some embodiments, the inhibitor is a rapamycin analog. In some embodiments, the inhibitor is a dual mTORC1/mTORC2 inhibitor (e.g., a catalytic and/or ATP-competitive inhibitor). In some embodiments, the inhibitor is a dual PI3k/mTOR inhibitor.

In some embodiments, the inhibitor of the mTOR pathway targets a receptor tyrosine kinase (RTK). In some embodiments, the receptor tyrosine kinase is encoded by genes EGFR (ERBB1) and/or HER2 (ERBB2).

In some embodiments, the inhibitor of the mTOR pathway is everolimus, rapamycin (sirolimus), and/or a rapamycin analog (rapalogs). In some embodiments, the inhibitor of the mTOR pathway is metformin. In some embodiments, the inhibitor of the mTOR pathway is an anti-aging drug, a senolytic (e.g., Azithromycin, Quercetin, doxycycline, chloroquine and/or chloroquine-related compound), and/or a NAD+ booster (e.g., conventional and/or investigational). In some embodiments, the inhibitor of the mTOR pathway is dactinomycin, mercaptopurine, melatonin, toremifene, emodin, and/or any combination thereof. See, for example, Zhavoronkov, “Geroprotective and senoremediative strategies to reduce the comorbidity, infection rates, severity, and lethality in gerophilic and gerolavic infections,” Aging 12(8) (2020); Sargiacomo et al., “COVID-19 and chronological aging: senolytics and other anti-aging drugs for the treatment or prevention of corona virus infection?” Aging 12(8) (2020); and Zhou et al., “Network-based drug repurposing for novel coronavirus 2019-nCoV/SARS-CoV-2,” Cell Discovery 6(14) (2020), each of which is hereby incorporated by reference herein in its entirety.

In some embodiments, the method comprises administering any combination of the abovementioned mTOR pathway inhibitors. In some embodiments, the inhibitor is administered as a therapeutic composition. In some embodiments, the administration of the inhibitor induces an upregulation or increased levels of α-Klotho, β-Klotho, and/or γ-Klotho. In some embodiments, the administration of the inhibitor improves outcomes for the subject diagnosed with COVID-19, SARS, and/or MERS. In some embodiments, the method further comprises co-administering a therapeutically effective amount of a Klotho polypeptide to the subject (e.g., α-Klotho, β-Klotho, and/or γ-Klotho).

Inhibitors of the NF-κB Pathway

As described above, studies have reported a link between inflammation to low Klotho expression and to accelerated aging. Furthermore, inflammation is a complication observed in relation to COVID-19 (e.g., cytokine storm). Thus, a treatment directed towards reducing the inflammatory response can ameliorate the symptoms of COVID-19, for example, by increasing Klotho levels. One of the inflammatory mediators implicated in the downregulation of Klotho expression is the NF-κB pathway, which is in turn promoted by tumor necrosis factor (TNF) and TNF-related weak inducer of apoptosis (TWEAK). See, Moreno et al., “The Inflammatory Cytokines TWEAK and TNFα Reduce Renal Klotho Expression through NFκB,” JASN 22(7) (2011), which is hereby incorporated by reference herein in its entirety.

As such, provided herein is a method for treating a severe acute respiratory syndrome-related coronavirus (SARS-CoV) infection in a subject in need thereof, the method comprising administering a therapeutically effective amount of an inhibitor of the NF-κB pathway.

In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the inhibitor of the NF-κB pathway targets any of the components and/or intermediates of the NF-κB pathway. In some such embodiments, the inhibitor of the NF-κB pathway induces an upregulation and/or reduces an inhibition of Klotho as a result of the targeting of any of the components and/or intermediates of the NF-κB pathway. For example, in some embodiments, the inhibitor of the NF-κB pathway targets a tumor necrosis factor receptor (TNF-R), an IκB kinase (IKK) complex (e.g., IKKα, IKKβ, and/or IKKγ (NEMO)), NF-κB-inducing kinase (NIK), ReIB, p100, and/or p52. In some embodiments, the inhibitor of the NF-κB pathway targets any one or more of the steps in the pathway. In some embodiments, the inhibitor of the NF-κB pathway targets the canonical or the non-canonical NF-κB pathway.

### Upstream Target Inhibitors

In some embodiments, the inhibitor of the NF-κB pathway is administered for the treatment and/or prophylaxis of the coronavirus infection in the subject. In some embodiments, the inhibitor of the NF-κB pathway is administered for the treatment and/or prophylaxis of a risk factor and/or complication of a coronavirus infection in the subject. In some embodiments, the inhibitor of the NF-κB pathway is administered for the treatment and/or prophylaxis of acute, mid-term and long-term clinical or health complications caused by a coronavirus infection in the subject. In some embodiments, the treatment comprises amelioration of symptoms of a coronavirus infection, a risk factor and/or complication of the coronavirus infection, and/or acute, midterm or long-term clinical or health complications caused by a coronavirus infection in the subject. In some embodiments, the treatment comprises a cure for a coronavirus infection, a risk factor and/or complication of the coronavirus infection, and/or acute, midterm or long-term clinical or health complications caused by a coronavirus infection in the subject. In some embodiments, the coronavirus infection is a severe acute respiratory syndrome-related coronavirus (SARS-CoV) infection in a subject. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the inhibitor of the NF-κB pathway targets a target that is upstream of the NF-κB pathway. In some embodiments, the upstream target inhibitor is Calagualine (fern derivative); Conophylline (Ervatamia microphylla); Evodiamine (Evodiae fructus component); Geldanamycin; Perrilyl alcohol; Protein-bound polysaccharide from basidiomycetes; Rocaglamides (Aglaia derivatives); 15-deoxy-prostaglandin J(2); Adenovirus ElA; NS5A (Hepatitis C virus); NS3/4A (HCV protease); Golli BG21 (product of myelin basic protein); NPM-ALK oncoprotein; MAST205; Erbin overexpression; Rituximab (anti-CD20 antibody); Kinase suppressor of ras (KSR2); PEDF (pigment epithelium derived factor); TNAP; Betaine; Desloratadine; LY29 and LY30; MOL 294 (small molecule); Pefabloc (serine protease inhibitor); Rhein; and/or Salmeterol, fluticasone propionate.

For example, in some embodiments, the inhibitor of the NF-κB pathway targets a tumor necrosis factor receptor (TNF-R). In some embodiments, the inhibitor is a member of the TRAF protein family. In some embodiments, the TRAF protein is a dominant negative mutant. In some embodiments the inhibitor is a kinase (e.g., NIK or MEKK1). In some embodiments, the kinase is a kinase-deficient or dominant negative mutant (e.g., a kinase-deficient or dominant negative mutant of NIK or MEKK1).

In some embodiments, the upstream target inhibitor of the NF-κB pathway is a natural product, chemical, metal, metabolite, synthetic compound, inorganic complex, antioxidant, small molecule, peptide, protein (e.g., cellular, viral, bacterial, and/or fungal) and/or a physical condition. In some embodiments, the upstream target inhibitor of the NF-κB pathway is any of the compounds listed in Gilmore and Herscovitch, “Inhibitors of NF-κB signaling: 785 and counting,” Oncogene 25 (2006), which is hereby incorporated by reference herein in its entirety for all purposes.

### IKK and IκB Phosphorylation Inhibitors

In some embodiments, the inhibitor of the NF-κB pathway is administered for the treatment and/or prophylaxis of the coronavirus infection in the subject. In some embodiments, the inhibitor of the NF-κB pathway is administered for the treatment and/or prophylaxis of a risk factor and/or complication of a coronavirus infection in the subject. In some embodiments, the inhibitor of the NF-κB pathway is administered for the treatment and/or prophylaxis of acute, mid-term and long-term clinical or health complications caused by a coronavirus infection in the subject. In some embodiments, the treatment comprises amelioration of symptoms of a coronavirus infection, a risk factor and/or complication of the coronavirus infection, and/or acute, midterm or long-term clinical or health complications caused by a coronavirus infection in the subject. In some embodiments, the treatment comprises a cure for a coronavirus infection, a risk factor and/or complication of the coronavirus infection, and/or acute, midterm or long-term clinical or health complications caused by a coronavirus infection in the subject. In some embodiments, the coronavirus infection is a severe acute respiratory syndrome-related coronavirus (SARS-CoV) infection in a subject. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the inhibitor of the NF-κB pathway targets phosphorylation of IκB and/or the IκB kinase (IKK) complex. In some embodiments, the IKK and/or IκB phosphorylation inhibitor is Lead; Anandamide; Artemisia vestita; Cobrotoxin; Dehydroascorbic acid (Vitamin C); Herbimycin A; Isorhapontigenin; Manumycin A; Pomegranate fruit extract; Tetrandine (plant alkaloid); Nitric oxide; Thienopyridine; Acetyl-boswellic acids; b-carboline; 1′-Acetoxychavicol acetate (Languas galanga); Apigenin (plant flavinoid); Cardamomin; Diosgenin; Furonaphthoquinone; Guggulsterone; Falcarindol; Honokiol; Hypoestoxide; Garcinone B; Kahweol; Kava (Piper methysticum) derivatives; g-mangostin (from Garcinia mangostana); N-acetylcysteine; Nitrosylcobalamin (vitamin B12 analog); Piceatannol; Plumbagin (5-hydroxy-2-methyl-1,4-naphthoquinone); Quercetin; Rosmarinic acid; Semecarpus anacardiu extract; Staurosporine; Sulforaphane and phenylisothiocyanate; Theaflavin (black tea component); Tilianin; g-Tocotrienol; Wedelolactone; Withanolides; Zerumbone; Silibinin; Betulinic acid; Ursolic acid; Monochloramine and glycine chloramine (NH2Cl); Anethole; Baoganning; Black raspberry extracts (cyanidin 3-O-glucoside, cyanidin 3-O-(2(G)-xylosylrutinoside), cyanidin 3-O-rutinoside); Buddlejasaponin IV; Cacospongionolide B; Calagualine; Carbon monoxide; Cardamonin; Cycloepoxydon; 1-hydroxy-2-hydroxymethyl-3-pent-1-enylbenzene; Decursin; Dexanabinol; Digitoxin; Diterpenes; Docosahexaenoic acid; Extensively oxidized low density lipoprotein (ox-LDL), 4-Hydroxynonenal (HNE); Flavopiridol; [6]-gingerol; casparol; Glossogyne tenuifolia; Guggulsterone; Indirubin-3′-oxime; Licorce extracts; Oleandrin; Omega 3 fatty acids; Panduratin A (from Kaempferia pandurata, Zingiberaceae); Petrosaspongiolide M; Pinosylvin; Plagius flosculosus extract polyacetylene spiroketal; Phytic acid (inositol hexakisphosphate); Pomegranate fruit extract; Prostaglandin A1; 20(S)-Protopanaxatriol (ginsenoside metabolite); Rengyolone; Rottlerin; Saikosaponin-d; Saline (low Na+ istonic); Salvia miltiorrhizae water-soluble extract; Sanguinarine (pseudochelerythrine, 13-methyl-[1,3]-benzodioxolo-[5,6-c]-1,3-dioxolo-4,5 phenanthridinium); Sesquiterpene lactones (parthenolide; ergolide; guaianolides); Scoparone; Silymarin; Sulindac; Vesnarinone; Xanthoangelol D; IKKb peptide to NEMO binding domain; NEMO CC2-LZ peptide; Adenovirus E3-14.7K; Adenovirus E3-10.4/14.5K; Core protein (Hepatitis C virus); E7 (Papillomavirus); MC160 (Mollusum Contagiosum virus); MC159 (Mollusum contagiosum virus); NS5B (Hepatitis C virus); vIRF3 (KSHV); Cytomegalovirus; HB-EGF (Heparin-binding epidermal growth factor-like growth factor); Hepatocyte growth factor; PAN1 (aka NALP2 or PYPAF2); PTEN (tumor suppressor); Interleukin-10; Anti-thrombin III; Chorionic gonadotropin; FHIT (Fragile histidine triad protein); Interferon-a; SOCS1; AGRO100 (G-quadruplex oligodeoxynucleotide); 2-amino-3-cyano-4-aryl-6-(2-hydroxy-phenyl)pyridine derivatives; Acrolein; AS602868; Aspirin, sodium salicylate; Dihydroxyphenylethanol; Epoxyquinone A monomer; Inhibitor 22; MLB120 (small molecule); Novel small-molecule inhibitor; BMS-345541; CYL-19s and CYL-26z, two synthetic alpha-methylene-gamma-butyrolactone derivatives; ACHP (2-amino-6-[2-(cyclopropylmethoxy)-6-hydroxyphenyl]-4-piperidin-4-yl nicotinonitrile; Compound A; Compound 5; Cyclopentenones; Jesterone dimer; PS-1145 (MLN1145); 2-[(aminocarbonyl)amino]-5-acetylenyl-3-thionphenecarboxamides; SC-514; (Amino)imidazolylcarboxaldehyde derivative; Amino-pyrimidine; Benzoimidazole derivative; CDDO-Me (synthetic triterpenoid); CHS 828 (anticancer drug); Diaylpyridine derivative; Imidazolylquinoline-carboxaldehyde derivative; Indolecarboxamide; LF15-0195 (analog of 15-deoxyspergualine); ML120B; MX781 (retinoid antagonist); NSAIDs; N-(4-hydroxyphenyl) retinamide; Pyrazolo[4,3-c]quinoline derivative; Pyridooxazinone derivative; Scytonemin; Survanta (Surfactant product); Sulfasalazine; Sulfasalazine analogs; Thalidomide; Azidothymidine (AZT); BAY-11-7082 (E3((4-methylphenyl)-sulfonyl)-2-propenenitrile); BAY-11-7083 (E3((4-t-butylphenyl)-sulfonyl)-2-propenenitrile); Benzyl isothiocyanate; Carboplatin; Gabexate mesilate; Gleevec (Imatanib); Hydroquinone; Ibuprofen; Inhaled isobutyl nitrite; Methotrexate; Monochloramine; Nafamostat mesilate; Statins (several); THI 52 (1-naphthylethyl-6,7-dihydroxy-1,2,3,4-tetrahydroisoquinoline); tetrahydroisoquinoline); 1,2,4-thiadiazolidine derivatives; YC-1; and/or Mild hypothermia.

For example, in some embodiments, the inhibitor of the NF-κB pathway targets an IκB kinase (IKK) complex. In some embodiments, the inhibitor targets IKKα, IKKβ, and/or IKKγ (NEMO). In some embodiments, the inhibitor is an ATP analog. In some embodiments, the inhibitor is a thiol-reactive compound that interacts with a cysteine residue on the target IKK. In some embodiments, the inhibitor is a dominant-negative mutant of IKKα, IKKβ, or IKKγ.

In some embodiments, the IKK and/or IκB phosphorylation inhibitor of the NF-κB pathway is a natural product, chemical, metal, metabolite, synthetic compound, inorganic complex, antioxidant, small molecule, peptide, protein (e.g., cellular, viral, bacterial, and/or fungal) and/or a physical condition. In some embodiments, the IKK and/or IκB phosphorylation inhibitor of the NF-κB pathway is any of the compounds listed in Gilmore and Herscovitch, “Inhibitors of NF-κB signaling: 785 and counting,” Oncogene 25 (2006), which is hereby incorporated by reference herein in its entirety for all purposes.

### IκB Degradation Inhibitors

In some embodiments, the inhibitor of the NF-κB pathway is administered for the treatment and/or prophylaxis of the coronavirus infection in the subject. In some embodiments, the inhibitor of the NF-κB pathway is administered for the treatment and/or prophylaxis of a risk factor and/or complication of a coronavirus infection in the subject. In some embodiments, the inhibitor of the NF-κB pathway is administered for the treatment and/or prophylaxis of acute, mid-term and long-term clinical or health complications caused by a coronavirus infection in the subject. In some embodiments, the treatment comprises amelioration of symptoms of a coronavirus infection, a risk factor and/or complication of the coronavirus infection, and/or acute, midterm or long-term clinical or health complications caused by a coronavirus infection in the subject. In some embodiments, the treatment comprises a cure for a coronavirus infection, a risk factor and/or complication of the coronavirus infection, and/or acute, midterm or long-term clinical or health complications caused by a coronavirus infection in the subject. In some embodiments, the coronavirus infection is a severe acute respiratory syndrome-related coronavirus (SARS-CoV) infection in a subject. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the inhibitor of the NF-κB pathway targets degradation of IκB. In some embodiments, the IκB degradation inhibitor is Zinc; Alachlor; Amentoflavone; Artemisia capillaris Thunb extract; Artemisia iwayomogi extract; L-ascorbic acid; Antrodia camphorata; Aucubin; Baicalein; b-lapachone; Blackberry extract; Buchang-tang; Capsaicin (8-methyl-N-vanillyl-6-nonenamide); Catalposide; Cyclolinteinone (sponge sesterterpene); Dihydroarteanniun; Docosahexaenoic acid; Emodin (3-methyl-1,6,8-trihydroxyanthraquinone); Ephedrae herba (Mao); Equol; Erbstatin (tyrosine kinase inhibitor); Estrogen (E2); Ethacrynic acid; Fosfomycin; Fungal gliotoxin; Gamisanghyulyunbueum; Genistein (tyrosine kinase inhibitor); Genipin; Glabridin; Glucosamine sulfate; Glutamine; Gumiganghwaltang; Isomallotochromanol and isomallotochromene; Kochia scoparia fruit (methanol extract); Leflunomide metabolite (A77 1726); Melatonin; 5′-methylthioadenosine; Midazolam; Momordin I; Mosla dianthera extract; Morinda officinalis extract; Opuntia ficus indica va saboten extract; b-Phenylethyl (PEITC) and 8-methylsulphinyloctyl isothiocyanates (MSO) (watercress); Platycodin saponins; Polymyxin B; Poncirus trifoliata fruit extract; Probiotics; Prostaglandin 15-deoxy-Δ(12,14)-PGJ(2); Resiniferatoxin; Stinging nettle (Urtica dioica) plant extracts; Thiopental; Tipifarnib; Titanium; TNP-470 (angiogenesis inhibitor); Trichomomas vaginalis infection; Triglyceride-rich lipoproteins; Ursodeoxycholic acid; Xanthium strumarium L. (methanol extract); Penetratin; Vasoactive intestinal peptide; K1L (Vaccinia virus protein); Nef (HIV-1); Vpu protein (HIV-1); g-glutamylcysteine synthetase; Heat shock protein-70; ST2 (IL-1-like receptor secreted form); YopJ (encoded by Yersinia pseudotuberculosis); Activated protein C (APC); a-melanocyte-stimulating hormone (a-MSH); IL-13; Intravenous immunoglobulin; Murr1 gene product; Neurofibromatosis-2 (NF-2; merlin) protein; Pituitary adenylate cyclase-activating polypeptide (PACAP); SAW (Saccharomyces boulardii anti-inflammatory factor); Acetaminophen; 1-Bromopropane; Diamide (tyrosine phosphatase inhibitor); Dobutamine; E-73 (cycloheximide analog); Ecabet sodium; Gabexate mesilate; Glimepiride; Hypochlorite; Losartin; LY294002 (PI3-kinase inhibitor) [2-(4-morpholinyl)-8-phenylchromone]; Pervanadate (tyrosine phosphatase inhibitor); Phenylarsine oxide (PAO, tyrosine phosphatase inhibitor); Phenytoin; Sabaeksan; U0126 (MEK inhibitor); Ro106-9920 (small molecule); Low level laser therapy; and/or Electrical stimulation of vagus nerve.

For example, in some embodiments, the inhibitor of the NF-κB pathway inhibits ubiquitination or proteasomal degradation of IκB. In some embodiments, the inhibitor is a peptide aldehyde, a cysteine protease inhibitor, a β-lactone, a dipeptidyl boronate, or a serine protease inhibitor.

In some embodiments, the IκB degradation inhibitor of the NF-κB pathway is a natural product, chemical, metal, metabolite, synthetic compound, inorganic complex, antioxidant, small molecule, peptide, protein (e.g., cellular, viral, bacterial, and/or fungal) and/or a physical condition. In some embodiments, the IκB degradation inhibitor of the NF-κB pathway is any of the compounds listed in Gilmore and Herscovitch, “Inhibitors of NF-κB signaling: 785 and counting,” Oncogene 25 (2006), which is hereby incorporated by reference herein in its entirety for all purposes.

### Proteasome and Protease Inhibitors

In some embodiments, the inhibitor of the NF-κB pathway is administered for the treatment and/or prophylaxis of the coronavirus infection in the subject. In some embodiments, the inhibitor of the NF-κB pathway is administered for the treatment and/or prophylaxis of a risk factor and/or complication of a coronavirus infection in the subject. In some embodiments, the inhibitor of the NF-κB pathway is administered for the treatment and/or prophylaxis of acute, mid-term and long-term clinical or health complications caused by a coronavirus infection in the subject. In some embodiments, the treatment comprises amelioration of symptoms of a coronavirus infection, a risk factor and/or complication of the coronavirus infection, and/or acute, midterm or long-term clinical or health complications caused by a coronavirus infection in the subject. In some embodiments, the treatment comprises a cure for a coronavirus infection, a risk factor and/or complication of the coronavirus infection, and/or acute, midterm or long-term clinical or health complications caused by a coronavirus infection in the subject. In some embodiments, the coronavirus infection is a severe acute respiratory syndrome-related coronavirus (SARS-CoV) infection in a subject. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the inhibitor of the NF-κB pathway targets a proteasome and/or a protease in the NF-κB pathway. In some embodiments, the proteasome and/or protease inhibitor is Lactacystine, b-lactone; Cyclosporin A; ALLnL (N-acetyl-leucinyl-leucynil-norleucynal, MG101); LLM (N-acetyl-leucinyl-leucynil-methional); Z-LLnV (carbobenzoxyl-leucinyl-leucynil-norvalinal,MG115); Z-LLL (N-carbobenzoxyl-L-leucinyl-L-leucinyl-L-norleucinal, MG132); Ubiquitin ligase inhibitors; Boronic acid peptide; PS-341 (Bortezomib); Salinosporamide A (1, NPI-0052); FK506 (Tacrolimus); Deoxyspergualin; Disulfiram; APNE (N-acetyl-DL-phenylalanine-b-naphthylester); BTEE (N-benzoyl L-tyrosine-ethylester); DCIC (3,4-dichloroisocoumarin); DFP (diisopropyl fluorophosphate); TPCK (N-a-tosyl-L-phenylalanine chloromethyl ketone); and/or TLCK (N-a-tosyl-L-lysine chloromethyl ketone).

In some embodiments, the proteasome and/or protease inhibitor of the NF-κB pathway is a natural product, chemical, metal, metabolite, synthetic compound, inorganic complex, antioxidant, small molecule, peptide, protein (e.g., cellular, viral, bacterial, and/or fungal) and/or a physical condition. In some embodiments, the proteasome and/or protease inhibitor of the NF-κB pathway is any of the compounds listed in Gilmore and Herscovitch, “Inhibitors of NF-κB signaling: 785 and counting,” Oncogene 25 (2006), which is hereby incorporated by reference herein in its entirety for all purposes.

### IκB a Upregulation, NF-κB Nuclear Translocation, and NF-κB Expression Inhibitors

In some embodiments, the inhibitor of the NF-κB pathway is administered for the treatment and/or prophylaxis of the coronavirus infection in the subject. In some embodiments, the inhibitor of the NF-κB pathway is administered for the treatment and/or prophylaxis of a risk factor and/or complication of a coronavirus infection in the subject. In some embodiments, the inhibitor of the NF-κB pathway is administered for the treatment and/or prophylaxis of acute, mid-term and long-term clinical or health complications caused by a coronavirus infection in the subject. In some embodiments, the treatment comprises amelioration of symptoms of a coronavirus infection, a risk factor and/or complication of the coronavirus infection, and/or acute, midterm or long-term clinical or health complications caused by a coronavirus infection in the subject. In some embodiments, the treatment comprises a cure for a coronavirus infection, a risk factor and/or complication of the coronavirus infection, and/or acute, midterm or long-term clinical or health complications caused by a coronavirus infection in the subject. In some embodiments, the coronavirus infection is a severe acute respiratory syndrome-related coronavirus (SARS-CoV) infection in a subject. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the inhibitor of the NF-κB pathway targets IκB a upregulation, NF-κB nuclear translocation, and/or NF-κB expression. In some embodiments, the IκB a upregulation, NF-κB nuclear translocation, and/or NF-κB expression inhibitor is Antrodia camphorata extract; Apigenin (4′,5,7-trihydroxyflavone); Glucocorticoids (dexamethasone, prednisone, methylprednisolone); Human breast milk; a-pinene; Agastache rugosa leaf extract; Alginic acid; Astragaloside IV; Atorvastatin; 2′,8″-biapigenin; Blue honeysuckle extract; Buthus martensi Karsch extract; Chiisanoside; 15-deoxyspergualin; Eriocalyxin B; Gangliosides; Harpagophytum procumbens (Devil's Claw) extracts; Hirsutenone; JM34 (benzamide derivative); KIOM-79 (combined plant extracts); Leptomycin B (LMB); Nucling; o,o′-bismyristoyl thiamine disulfide (BMT); Oregonin; 1,2,3,4,6-penta-O-galloyl-b-D-glucose; Platycodi radix extract; Phallacidin; Piperine; Pitavastatin; Probiotics; Rhubarb aqueous extract; Selenomethionine; Salvia miltiorrhoza Bunge extract; ShenQi compound recipe; Sophorae radix extract; Sopoongsan; Sphondin (furanocoumarin derivative from Heracleum laciniatum); Younggaechulgam-tang; Clarithromycin; 5F (from Pteri syeminpinnata L)); AT514 (serratamolide); oxidized 1-palmitoyl-2-arachidonoyl-sn-glycero-3-phosphorylcholine (OXPAPC); Sorbus commixta cortex (methanol extract); Cantharidin; Cornus officinalis extract; Neomycin; Paeoniflorin; Rapamycin; Sargassum hemiphyllum methanol extract; Shenfu; Tripterygium polyglycosides; PN50; Cell permeable NLS peptides; RelA peptides (P1 and P6); Canine Distemper Virus; MNF (myxoma virus); 3C protease (encephalomyocarditis virus); ZUD protein; HSCO (hepatoma protein); b-amyloid protein; Surfactant protein A (SP-A); DQ 65-79 (aa 65-79 of the alpha helix of the alpha-chain of the class II HLA molecule DQA03011); C5a; IL-10; IL-11; IL-13; Foxlj; Glucorticoid-induced leucine zipper protein (GILZ); Heat shock protein 72; Retinoic acid receptor-related orphan receptor-alpha; TAT-SR-IkBa; MTS—SR-IkBa; p105-SR; ZAS3 protein; RASSF1A gene overexpression; Onconase (Ranpirnase); R-etodolac; BMD (N(1)-Benzyl-4-methylbenzene-1,2-diamine); Carbaryl; Indole-3-carbinol; Dioxin; Dehydroxymethylepoxyquinomicin (DHMEQ); Dipyridamole; Disulfiram; Diltiazem; Fluvastatin; JSH-23 (4-Methyl-(3-phenyl-propyl)-benzene-1,2-diamine; KL-1156 (6-Hydroxy-7-methoxychroman-2-carboxylic acid phenylamide); Leflunomide; Levamisole; MEB (2-(4-morpholynl) ethyl butyrate hydrochloride); Rolipram; SC236 (a selective COX-2 inhibitor); Triflusal; Volatile anesthetic treatment; Moxifloxacin; Omapatrilat, enalapril, CGS 25462; and/or Estrogen enhanced transcript.

For example, in some embodiments, the inhibitor of the NF-κB pathway inhibits nuclear translocation of NF-κB. In some embodiments, the inhibitor is a cell-permeable peptide.

In some embodiments, the IκB a upregulation, NF-κB nuclear translocation, and/or NF-κB expression inhibitor of the NF-κB pathway is a natural product, chemical, metal, metabolite, synthetic compound, inorganic complex, antioxidant, small molecule, peptide, protein (e.g., cellular, viral, bacterial, and/or fungal) and/or a physical condition. In some embodiments, the IκB a upregulation, NF-κB nuclear translocation, and/or NF-κB expression inhibitor of the NF-κB pathway is any of the compounds listed in Gilmore and Herscovitch, “Inhibitors of NF-κB signaling: 785 and counting,” Oncogene 25 (2006), which is hereby incorporated by reference herein in its entirety for all purposes.

### NF-κB DNA-Binding Inhibitors

In some embodiments, the inhibitor of the NF-κB pathway is administered for the treatment and/or prophylaxis of the coronavirus infection in the subject. In some embodiments, the inhibitor of the NF-κB pathway is administered for the treatment and/or prophylaxis of a risk factor and/or complication of a coronavirus infection in the subject. In some embodiments, the inhibitor of the NF-κB pathway is administered for the treatment and/or prophylaxis of acute, mid-term and long-term clinical or health complications caused by a coronavirus infection in the subject. In some embodiments, the treatment comprises amelioration of symptoms of a coronavirus infection, a risk factor and/or complication of the coronavirus infection, and/or acute, midterm or long-term clinical or health complications caused by a coronavirus infection in the subject. In some embodiments, the treatment comprises a cure for a coronavirus infection, a risk factor and/or complication of the coronavirus infection, and/or acute, midterm or long-term clinical or health complications caused by a coronavirus infection in the subject. In some embodiments, the coronavirus infection is a severe acute respiratory syndrome-related coronavirus (SARS-CoV) infection in a subject. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the inhibitor of the NF-κB pathway targets NF-κB DNA-binding. In some embodiments, the NF-κB DNA-binding inhibitor is a metal (chromium, cadmium, gold, lead, mercury, zinc, arsenic); Actinodaphine (from Cinnamomum insularimontanum); Anthocyanins (soybean); Arnica montana extract (sequiterpene lactones); Artemisinin; Baicalein (5,6,7-trihydroxyflavone); Bambara groundnut (Vignea subterranean); b-lapachone (1,2-naphthoquinone); Biliverdin; Brazilian; Calcitriol (1a,25-dihydroxyvitamin D3); Campthothecin; Cancer bush (Sutherlandia frutescens); Capsiate; Catalposide (stem bark); Cat's claw bark (Uncaria tomentosa; Rubiaceae); Maca; Cheongyeolsaseuptang; Chitosan; Chicory root (guaianolide 8-deoxylactucin); Chondrotin sulfate proteoglycan degradation product; Clarithromycin; Cloricromene; Compound K (from Panax ginseng); Cortex cinnamomi extract; Cryptotanshinone; Cytochalasin D; DA-9201 (from black rice); Danshenshu; Diterpenoids from Isodon rubescens or Liverwort Jungermannia; ent-kaurane diterpenoids (Croton tonkinensis leaves); Epinastine hydrochloride; Epoxyquinol A (fungal metabolite); Erythromycin; Evodiamine; Fish oil feeding; Fomes fomentarius methanol extracts; Fucoidan; Gallic acid; Ganoderma lucidum (fungal dried spores or fruting body); Garcinol (fruit rind of Garcinia spp); Geranylgeraniol; Ginkgolide B; Glycyrrhizin; Halofuginone; Hematein (plant compound); Herbal compound 861; Hydroxyethyl starch; Hydroxyethylpuerarin; Hypericin; Kamebakaurin; Linoleic acid; Lithospermi radix; Macrolide antibiotics; Mediterranean plant extracts; 2-methoxyestradiol; 6-(Methylsulfinyl)hexyl isothiocyanate (Wasabi); Nicotine; Ochna macrocalyx bark extracts; Oridonin (diterpenoid from Rabdosia rubescens); PC-SPES (8 herb mixture); 1,2,3,4,6-penta-O-galloyl-b-D-glucose; Pepluanone; Phyllanthus amarus extracts; Plant compound A (a phenyl aziridine precursor); Polyozellin; Prenylbisabolane 3; Prostaglandin E2; Protein-bound polysaccharide (PSK); Quinic acid; Sanggenon C; Sesamin (from sesame oil); Shen-Fu; Silibinin; Sinomenine; Sword brake fern extract; Tanacetum larvatum extract; Tansinones (Salvia miltiorrhiza Bunge, Labiatae roots); Taurine+niacine; Thiazolidinedione MCC-555; Trichostatin A; Triptolide (PG490, extract of Chinese herb); Tyrphostin AG-126; Ursolic acid; Withaferin A; Xanthohumol (a hops prenylflavonoid); Xylitol; Yan-gan-wan; Yin-Chen-Hao; Yucca schidigera extract; Ghrelin; Peptide YY; Rapamycin; A238L IkB-like protein (African Swine Fever virus); C+V proteins (Sendai virus); E1B (Adenovirus); ICP27 (Herpes simplex virus-1); H4/N5 (Microplitis demolitor bracovirus); NS3/4A (Hepatitis C); Adiponectin; AIM2 (Absent in melanoma protein) overexpression; Angiopoietin-1; Antithrombin; AvrA protein (Salmonella); b-catenin; Bromelain; Calcium/calmodulin-dependent kinase kinase (CaMKK) (and increased intracellular calcium by ionomycin, UTP and thapsigargin); CD43 overexpression; FLN29 overexpression; FLICE-Like Inhibitory Protein (FLIP); G-120 (Ulmus davidiana Nakai glycoprotein); Gax (homeobox protein); HIV-1 Resistance Factor; Insulin-like growth factor binding protein-3; Interleukin 4 (IL-4); Leucine-rich effector proteins of Salmonella & Shigella (SspH1 and IpaH9.8); NDPP1 (CARD protein); Overexpressed ZIP1; p8; p202a (nterferon inducible protein); p21 (recombinant); PIAS1 (protein inhibitor of activatated STAT1); Pro-opiomelanocortin; PYPAF1 protein; Raf Kinase Inhibitor Protein (RKIP); Rhus verniciflua Stokes fruits 36 kDa glycoprotein; Secretory leucoprotease inhibitor (SLPI); Siah2; SIRT1 Deacetylase overexpression; Siva-1; Solana nigrum L.; Surfactant protein A; TomI (target of Myb-1) overexpression; Transdominant p50; Uteroglobin; Vascular endothelial growth factor (VEGF); ADP ribosylation inhibitors (nicotinamide, 3-aminobenzamide); 7-amino-4-methylcoumarin; Amrinone; Atrovastat (HMG-CoA reductase inhibitor); Benfotiamine (thiamine derivative); Bisphenol A; Caprofen; Carbocisteine; Celecoxib and germcitabine; Cinnamaldehyde, 2-methoxycinnamaldehyde, 2-hydroxycinnamaldehyde; Commerical peritoneal dialysis solution; CP Compound (6-Hydroxy-7-methoxychroman-2-carboxylic acid phenylamide); Cyanoguanidine CHS 828; (kB site) Decoy oligonucleotides; Diarylheptanoid 7-(4′-hydroxy-3′-methoxyphenyl)-1-phenylhept-4-en-3-one; a-difluoromethylornithine (polyamine depletion); DTD (4,10-dichloropyrido[5,6:4,5]thieno[3,2-d′:3,2-d]-1, 2, 3-ditriazine); Evans Blue; Evodiamine; Fenoldopam; Fexofenadine hydrochloride; Fibrates; FK778; Flunixin meglumine; Flurbiprofen; Hydroquinone (HQ); IMD-0354; JSH-21 (N1-Benzyl-4-methylbenzene-1,2-diamine); KT-90 (morphine synthetic derivative); Lovastatin; Mercaptopyrazine; Mevinolin, 5′-methylthioadenosine (MTA); Monomethylfumarate; Moxifloxacin; Nicorandil; Nilvadipine; Nitric oxide-donating aspirin; Panepoxydone; Peptide nucleic acid-DNA decoys; Perindopril; 6(5H)-phenanthridinone and benzamide; Phenyl-N-tert-butylnitrone (PBN); Pioglitazone (PPARgamma ligand); Pirfenidone; Pyridine N-oxide derivatives; Quinadril (ACE inhibitor); Raloxifene; Raxofelast; Ribavirin; Rifamides; Ritonavir; Rosiglitazone; Roxithromycin; Santonin diacetoxy acetal derivative; Serotonin derivative (N-(p-coumaroyl) serotonin, SC); Simvastatin; SM-7368 (small molecule); T-614 (a methanesulfoanilide anti-arthritis inhibitor); Sulfasalazine; SUN C8079; Triclosan plus cetylpyridinium chloride; Tobacoo smoke; Verapamil; Heat (fever-like); Hypercapnic acidosis; Hyperosmolarity; Hypothermia; and/or Moderate alcohol intake.

For example, in some embodiments, the inhibitor of the NF-κB pathway inhibits DNA binding of NF-κB. In some embodiments, the inhibitor is a sesquiterpene lactone.

In some embodiments, the NF-κB DNA-binding inhibitor of the NF-κB pathway is a natural product, chemical, metal, metabolite, synthetic compound, inorganic complex, antioxidant, small molecule, peptide, protein (e.g., cellular, viral, bacterial, and/or fungal) and/or a physical condition. In some embodiments, the NF-κB DNA-binding inhibitor of the NF-κB pathway is any of the compounds listed in Gilmore and Herscovitch, “Inhibitors of NF-κB signaling: 785 and counting,” Oncogene 25 (2006), which is hereby incorporated by reference herein in its entirety for all purposes.

### NF-κB Transactivation Inhibitors

In some embodiments, the inhibitor of the NF-κB pathway is administered for the treatment and/or prophylaxis of the coronavirus infection in the subject. In some embodiments, the inhibitor of the NF-κB pathway is administered for the treatment and/or prophylaxis of a risk factor and/or complication of a coronavirus infection in the subject. In some embodiments, the inhibitor of the NF-κB pathway is administered for the treatment and/or prophylaxis of acute, mid-term and long-term clinical or health complications caused by a coronavirus infection in the subject. In some embodiments, the treatment comprises amelioration of symptoms of a coronavirus infection, a risk factor and/or complication of the coronavirus infection, and/or acute, midterm or long-term clinical or health complications caused by a coronavirus infection in the subject. In some embodiments, the treatment comprises a cure for a coronavirus infection, a risk factor and/or complication of the coronavirus infection, and/or acute, midterm or long-term clinical or health complications caused by a coronavirus infection in the subject. In some embodiments, the coronavirus infection is a severe acute respiratory syndrome-related coronavirus (SARS-CoV) infection in a subject. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the inhibitor of the NF-κB pathway targets NF-κB transactivation. In some embodiments, the NF-κB transactivation inhibitor is 8-acetoxy-5-hydroxyumbelliprenin; Adenosine and cyclic AMP; Artemisia sylvatica sesquiterpene lactones; a-zearalenol; BSASM (plant extract mixture); Bifodobacteria; Bupleurum fruticosum phenylpropanoids; Blueberry and berry mix (Optiberry); 4′-demethyl-6-methoxypodophyllotoxin (lignan of Linum tauricum Willd. ssp. tauricum); Cycloprodigiosin hycrochloride; Eckol/Dieckol (seaweed E stolonifera); Extract of the stem bark of Mangifera indica L.; Fructus Benincasae Recens extract; Glucocorticoids (dexametasone, prednisone, methylprednisolone); Gypenoside XLIX (from Gynostemma pentaphyllum); Kwei Ling Ko (Tortoise shell-Rhizome jelly); Ligusticum chuanxiong Hort root; Luteolin; Manassantins A and B; Mesuol; Nobiletin; 4-phenylcoumarins (from Marila pluricostata); Phomol; Psychosine; Qingkailing and Shuanghuanglian (Chinese medicinal preparations); Saucerneol D and saucerneol E; Smilax bockii warb extract (flavenoids); Trilinolein; Uncaria tomentosum plant extract; Witheringia solanacea leaf extracts; Wortmannin (fungal metabolite); BZLF1 (EBV protein); SH gene products (Paromyxovirus); NRF (NF-kB repression factor); PIAS3; PTX-B (pertussis toxin binding protein); Antithrombin; 17-allylamino-17-demethoxygeldanamycin; 6-aminoquinazoline derivatives; Chromene derivatives; D609 (phosphatidylcholine-phospholipase C inhibitor); Dimethylfumarate (DMF); Ethyl 2-[(3-methyl-2,5-dioxo(3-pyrrolinyl)) pyrimidine-5-carboxylate pyrimidine-5-carboxylate; Histidine; HIV-1 protease inhibitors (nelfinavir, ritonavir, or saquinavir); Phenethylisothiocyanate; Pranlukast; RO31-8220 (PKC inhibitor); SB203580 (p38 MAPK inhibitor); Tetrathiomolybdate; Tranilast [N-(3,4-dimethoxycinnamoyl)anthranilic acid]; 3,4,5-trimethoxy-4′-fluorochalcone; Troglitazone; 9-aminoacridine (9AA) derivatives (including the antimalaria drug quinacrine); Mesalamine; and/or Low gravity.

For example, in some embodiments, the inhibitor of the NF-κB pathway inhibits transcriptional activation of NF-κB. In some embodiments, the inhibitor selectively inhibits phosphatidylcholine-phospholipase C inhibitor, protein kinase C or p38 MAPK. In some embodiments, the inhibitor of the NF-κB pathway is an inhibitor of xB (e.g., IκB).

In some embodiments, the NF-κB transactivation inhibitor of the NF-κB pathway is a natural product, chemical, metal, metabolite, synthetic compound, inorganic complex, antioxidant, small molecule, peptide, protein (e.g., cellular, viral, bacterial, and/or fungal) and/or a physical condition. In some embodiments, the NF-κB transactivation inhibitor of the NF-κB pathway is any of the compounds listed in Gilmore and Herscovitch, “Inhibitors of NF-κB signaling: 785 and counting,” Oncogene 25 (2006), which is hereby incorporated by reference herein in its entirety for all purposes.

### Antioxidants

In some embodiments, the inhibitor of the NF-κB pathway is administered for the treatment and/or prophylaxis of the coronavirus infection in the subject. In some embodiments, the inhibitor of the NF-κB pathway is administered for the treatment and/or prophylaxis of a risk factor and/or complication of a coronavirus infection in the subject. In some embodiments, the inhibitor of the NF-κB pathway is administered for the treatment and/or prophylaxis of acute, mid-term and long-term clinical or health complications caused by a coronavirus infection in the subject. In some embodiments, the treatment comprises amelioration of symptoms of a coronavirus infection, a risk factor and/or complication of the coronavirus infection, and/or acute, midterm or long-term clinical or health complications caused by a coronavirus infection in the subject. In some embodiments, the treatment comprises a cure for a coronavirus infection, a risk factor and/or complication of the coronavirus infection, and/or acute, midterm or long-term clinical or health complications caused by a coronavirus infection in the subject. In some embodiments, the coronavirus infection is a severe acute respiratory syndrome-related coronavirus (SARS-CoV) infection in a subject. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the inhibitor of the NF-κB pathway is an antioxidant. In some embodiments, the inhibitor is Aged garlic extract (allicin); 2-Amino-1-methyl-6-phenylimidazo[4,5-b]pyridine (PhIP); Anetholdithiolthione; Apocynin; Apple juice/extracts; Aretemisa p7F (5,6,3′,5′-tetramethoxy 7,4′-hydroxyflavone); Astaxanthin; Benidipine; bis-eugenol; Bruguiera gymnorrhiza compounds; Butylated hydroxyanisole (BHA); Caffeic Acid Phenethyl Ester (3,4-dihydroxycinnamic acid, CAPE); Carnosol; b-Carotene; Carvedilol; Catechol derivatives; Celasterol; Cepharanthine; Chlorophyllin; Chlorogenic acid; Cocoa polyphenols; Curcumin (Diferulolylmethane); Dehydroevodiamine; Dehydroepiandrosterone (DHEA) and DHEA-sulfate (DHEAS); Dibenzylbutyrolactone lignans; Diethyldithiocarbamate (DDC); Diferoxamine; Dihydroisoeugenol; Dihydrolipoic acid; Dilazep+fenofibric acid; Dimethyldithiocarbamates (DMDTC); Dimethylsulfoxide (DMSO); Disulfiram; Ebselen; Edaravone; EPC-K1 (phosphodiester compound of vitamin E and vitamin C); Epigallocatechin-3-gallate (EGCG; green tea polyphenols); Ergothioneine; Ethyl pyruvate (glutathione depletion); Ethylene glycol tetraacetic acid (EGTA); Extract of the stem bark of Mangifera indica L.; Flavonoids (Crataegus; Boerhaavia diffusa root; xanthohumol); Gamma-glutamylcysteine synthetase (gamma-GCS); Ganoderma lucidum polysaccharides; Garcinol (from extract of Garcinia indica fruit rind); Ginkgo biloba extract; Glutathione; Hematein; Hydroquinone; 23-hydroxyursolic acid; IRFI 042 (Vitamin E-like compound); Iron tetrakis; Isovitexin; Kangen-karyu extract; Ketamine; L-cysteine; Lacidipine; Lazaroids; Ligonberries; a-lipoic acid; Lupeol; Magnolol; Maltol; Manganese superoxide dismutase (Mn-SOD); Mangiferin; Melatonin; Mulberry anthocyanins; Myricetin; Naringin; N-acetyl-L-cysteine (NAC); Nacyselyn (NAL); N-ethyl-maleimide (NEM); Nitrosoglutathione; Nordihydroguaiaritic acid (NDGA); Ochnaflavone; Orthophenanthroline; PMC (2,2,5,7,8-pentamethyl-6-hydroxychromane); Pentoxyifylline (1-(5′-oxohexyl) 3,7-dimehylxanthine, PTX); Phenolic antioxidants (Hydroquinone and tert-butyl hydroquinone); Phenylarsine oxide (PAO, tyrosine phosphatase inhibitor); Phyllanthus urinaria; Pyrithione; Pyrrolinedithiocarbamate (PDTC); Quercetin (low concentrations); Quinozolines; Rebamipide; Red wine; Ref-1 (redox factor 1); Resveratrol; Rg(3), a ginseng derivative; Rotenone; Roxithromycin; S-allyl-cysteine (SAC, garlic compound); Sauchinone; Spironolactone; Strawberry extracts; Taxifolin; Tempol; Tepoxaline (5-(4-chlorophenyl)-N-hydroxy-(4-methoxyphenyl)-N-methyl-iH-pyrazole-3-propanamide); Tetracylic A; a-tocopherol; a-torphryl acetate; a-torphryl succinate; Vitamin C; Vitamin B6; Vitamin D; Vitamin E derivatives; Wogonin (5,7-dihydroxy-8-methoxyflavone); and/or Yakuchinone A and B.

In some embodiments, the proteasome and/or protease inhibitor of the NF-κB pathway is a natural product, chemical, metal, metabolite, synthetic compound, inorganic complex, antioxidant, small molecule, peptide, protein (e.g., cellular, viral, bacterial, and/or fungal) and/or a physical condition. In some embodiments, the proteasome and/or protease inhibitor of the NF-κB pathway is any of the compounds listed in Gilmore and Herscovitch, “Inhibitors of NF-κB signaling: 785 and counting,” Oncogene 25 (2006), which is hereby incorporated by reference herein in its entirety for all purposes.

In some embodiments, the method comprises administering any combination of the abovementioned NF-κB pathway inhibitors. In some embodiments, the inhibitor is administered as a therapeutic composition. In some embodiments, the administration of the inhibitor induces an upregulation or increased levels of α-Klotho, β-Klotho, and/or γ-Klotho. In some embodiments, the administration of the inhibitor improves outcomes for the subject diagnosed with COVID-19, SARS, and/or MERS. In some embodiments, the method further comprises co-administering a therapeutically effective amount of a Klotho polypeptide (e.g., α-Klotho, 3-Klotho, and/or γ-Klotho) to the subject. In some embodiments, the method further comprises co-administering a therapeutically effective amount of an inhibitor of the mTOR pathway to the subject. In some embodiments, the method further comprises co-administering a therapeutically effective amount of a lipid-reducing compound to the subject. In some embodiments, the method further comprises co-administering a therapeutically effective amount of a statin to the subject.

Lipid-Lowering Agents

Analysis of COVID-19 infection data indicates an association between dyslipidemia and hyperlipidemia and an enhanced risk of severe manefestations of COVID-19. For instance, COVID-19 patients with high low-density lipoprotein (LDL) levels are at increased risk for severe symptoms of COVID-19, suggesting that treatment of the underlying dyslipidemia will lessen the effects of COVID-19. See, for example, Hariyanto and Kurniawan, “Dyslipidemia is associated with severe coronavirus disease 2019 (COVID-19) infection,” Diabetes Metab Syndr 14(5) (2020), which is hereby incorporated by reference herein in its entirety.

Notably, high levels of LDL are also tied to decreased Klotho expression, activation of the NF-κB pathway, and kidney injury, highlighting a consistent correlation with previously described COVID-19 risk factors and complications. Specifically, NF-κB and extracellular signal-regulated kinases (ERK) have been shown to regulate oxidized LDL, which in turn decreases Klotho mRNA and protein expression. Conversely, NF-κB and ERK inhibitors prevent ox-LDL-mediated Klotho downregulation. See, Sastre et al., “Hyperlipidemia-Associated Renal Damage Decreases Klotho Expression in Kidneys from ApoE Knockout Mice,” PLoS One 8(12) (2013), which is hereby incorporated by reference herein in its entirety. As such, what is needed in the art are methods for treating COVID-19 infection by reducing lipid levels in a patient in need thereof.

Provided herein is a method for treating a severe acute respiratory syndrome-related coronavirus (SARS-CoV) infection in a subject with hyperlipidemia and in need thereof, the method comprising administering a therapeutically effective amount of a lipid-reducing compound.

In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the lipid is a low-density lipoprotein (LDL), a high-density lipoprotein (HDL), triglyceride, and/or lipoprotein(a).

In some embodiments, the lipid-reducing compound is a statin, bile acid sequestrant, PCSK9 inhibitor, and/or fibrate. In some embodiments, the lipid-reducing compound is ezetimibe, niacin, lomitapide, bempedoic acid, mipomersen, sebelipase, glybera, volanesorsen, evinacumab, or lecithin. In some embodiments, the lipid-reducing compound is an HDL-based peptide. See, for example, Hegele and Tsimikas, “Lipid-Lowering Agents: Targets Beyond PCSK9,” Circulation Res 124(3) (2019), which is hereby incorporated by reference herein in its entirety.

In some embodiments, the subject was not previously treated with a lipid-reducing compound. In some embodiments, the subject was previously treated with a lipid-reducing compound, and the administering a therapeutically effective amount of the lipid-reducing compound includes increasing the dosage of the compound.

In some embodiments, the method comprises administering any combination of the abovementioned lipid-reducing compounds. In some embodiments, the lipid-reducing compound is administered as a therapeutic composition. In some embodiments, the administration of the lipid-reducing compound induces an upregulation or increased levels of α-Klotho, β-Klotho, and/or γ-Klotho. In some embodiments, the administration of the lipid-reducing compound improves outcomes for the subject diagnosed with COVID-19, SARS, and/or MERS. In some embodiments, the method further comprises co-administering a therapeutically effective amount of a Klotho polypeptide (e.g., α-Klotho, β-Klotho, and/or γ-Klotho) to the subject. In some embodiments, the method further comprises co-administering a therapeutically effective amount of an inhibitor of the mTOR pathway to the subject. In some embodiments, the method further comprises co-administering a therapeutically effective amount of an inhibitor of the NF-κB pathway to the subject. In some embodiments, the method further comprises co-administering a therapeutically effective amount of a statin to the subject.

In one embodiment, the method comprises treating a coronavirus infection in a subject in need thereof, the method comprising administering a therapeutically effective amount of a statin. In some embodiments, the subject has dyslipidemia or hyperlipidemia. In some embodiments, the subject is diagnosed with high cholesterol. In some embodiments, the coronavirus infection is a severe acute respiratory syndrome-related coronavirus (SARS-CoV) infection. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS. For example, in some embodiments, the dyslipidemia and/or hyperlipidemia in the subject is a risk factor for contracting the coronavirus infection (e.g., SARS-CoV-2, SARS-CoV-1, and/or MERS-CoV). In some embodiments, the dyslipidemia and/or hyperlipidemia in the subject is a risk factor for developing severe coronavirus infection (e.g., SARS-CoV-2, SARS-CoV-1, and/or MERS-CoV).

In some embodiments, the statin administered for treatment or prophylaxis of a coronavirus-mediated disease is rosuvastatin, atorvastatin, fluvastatin, lovastatin, pitavastatin, pravastatin, or simvastatin, a pharmaceutically acceptable salt thereof, or a combination thereof. In some embodiments, the statin is co-administered with another lipid-lowering drug (e.g., ezetimibe, niacin, lomitapide, bempedoic acid, mipomersen, sebelipase, glybera, volanesorsen, evinacumab, or lecithin). For example, in some embodiments, the combination is atorvastatin/ezetimibe (e.g., LIPTRUZET®), lovastatin/niacin (e.g., ADVICOR®), simvastatin/ezetimibe (e.g., VYTORIN®), or simvastatin/niacin (e.g., SIMCOR®).

In some embodiments, the statin administered is a prodrug. As used herein, a prodrug refers to a pharmaceutical composition that includes a biologically inactive compound that is metabolized in vivo to generate the active form of the drug. For instance, in some embodiments, the prodrug statin is rosuvastatin, atorvastatin, fluvastatin, lovastatin, pitavastatin, pravastatin, or simvastatin.

In some embodiments, the statin composition includes rosuvastatin (e.g., CRESTOR®) as an active ingredient. In some embodiments, the statin composition includes a compound disclosed in U.S. Pat. Nos. 6,316,460 or 6,858,618, each of which is hereby incorporated by reference, as an active ingredient. In some embodiments, the statin composition includes atorvastatin (e.g., LIPITOR®) as an active ingredient. In some embodiments, the statin composition includes fluvastatin (e.g., LESCOL® or LESCOL XL®) as an active ingredient. In some embodiments, the statin composition includes a compound disclosed in U.S. Pat. No. 6,242,003, which is hereby incorporated by reference, as an active ingredient.

In some embodiments, the statin composition includes lovastatin (e.g., ALTOPREV®) as an active ingredient. In some embodiments, the statin composition includes pitavastatin (e.g., LIVALO®) as an active ingredient. In some embodiments, the statin composition includes a compound disclosed in U.S. Pat. Nos. 5,856,336, 7,022,713, or 8,557,993, each of which is hereby incorporated by reference, as an active ingredient. In some embodiments, the statin composition includes pravastatin (e.g., PRAVACHOL®) as an active ingredient. In some embodiments, the statin composition includes simvastatin (e.g., ZOCOR®) as an active ingredient.

In some embodiments, the statin composition includes a compound described in Lee et al., 2007, “Comparison of Efficacy and Tolerability of Pitavastatin and Atorvastatin: an 8-Week, Multicenter, Randomized, Open-Label, Dose-Titration Study in Korean Patients with Hypercholesterolemia,” Clin Ther. 2007; 29:2365-73; Bradford et al., 1990, “Expanded clinical evaluation of lovastatin (EXCEL) study design and patient characteristics of a double blind, placebo controlled study in patients with moderate hypercholesterolemia. American Journal of Cardiology 66: p.44B-55B; Serruys et al., 2002, “Fluvastatin for Prevention of Cardiac Events Following Successful First Percutaneous Coronary Intervention: A Randomized Controlled Trial.,” JAMA 287:p.3215-3222; Sacks et al. 1996, “The effect of pravastatin on coronary events after myocardial infarction in patients with average cholesterol levels. Cholesterol and Recurrent Events Trial investigators,” New England Journal of Medicine, 1996. 335(14): p. 001-9; Anonymous, 2002 “Heart Protection Study Collaborative Group, MRC/BHF Heart Protection Study of cholesterol lowering with simvastatin in 20,536 high-risk individuals: a randomised placebo-controlled trial,” Lancet 360: p. 7-22; Jones et al., 2003, “Comparison of the efficacy and safety of rosuvastatin versus atorvastatin, simvastatin, and pravastatin across doses (STELLAR Trial), “Am J Cardiol. 92 (2): 152-60 each of which is hereby incorporated by reference herein in its entirety.

In some embodiments, a method is provided for treating or preventing a disease caused by a severe acute respiratory syndrome-related coronavirus (SARS-CoV) infection by administering a statin to a subject, e.g., with dyslipidemia or hyperlipidemia. In some embodiments, the coronavirus infection is a severe acute respiratory syndrome-related coronavirus (SARS-CoV) infection. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the treatment of the coronavirus infection comprises prevention of the coronavirus infection (e.g., prophylaxis for a coronavirus infection such as SARS-CoV-2, SARS-CoV-1, and/or MERS-CoV). In some embodiments, the treatment comprises amelioration of symptoms of a coronavirus infection (e.g., SARS-CoV-2, SARS-CoV-1, and/or MERS-CoV). In some embodiments, the treatment comprises a cure for a coronavirus infection (e.g., SARS-CoV-2, SARS-CoV-1, and/or MERS-CoV).

In some embodiments, the statin administered for the treatment of the coronavirus infection in the subject is rosuvastatin, atorvastatin, fluvastatin, lovastatin, pitavastatin, pravastatin, or simvastatin, and/or any combination or pharmaceutically acceptable salt thereof. In some embodiments, the statin administered for the treatment of the coronavirus infection in the subject is co-administered with another lipid-lowering drug (e.g., ezetimibe, niacin, lomitapide, bempedoic acid, mipomersen, sebelipase, glybera, volanesorsen, evinacumab, or lecithin). For example, in some embodiments, the statin administered for the treatment of the coronavirus infection in the subject is Atorvastatin/Ezetimibe (LIPTRUZET®), Lovastatin+Niacin (ADVICOR®), Simvastatin/Ezetimibe (VYTORIN®), or Simvastatin/Niacin-ER (SIMCOR®).

In some embodiments, the statin administered for the treatment of the coronavirus infection in the subject is a prodrug. As used herein, a prodrug refers to a pharmaceutical composition that includes a biologically inactive compound that is metabolized in vivo to generate the active form of the drug. For instance, in some embodiments, the prodrug statin is rosuvastatin, atorvastatin, fluvastatin, lovastatin, pitavastatin, pravastatin, or simvastatin.

In some embodiments, the statin to be administered for the treatment of the coronavirus infection in the subject is in the form of a statin therapeutic composition comprising an active ingredient (e.g., rosuvastatin, atorvastatin, fluvastatin, lovastatin, pitavastatin, pravastatin, and/or simvastatin), or a combination of active ingredients and/or a pharmaceutically acceptable salt thereof.

For example, in some embodiments, the statin therapeutic composition for the treatment of the coronavirus infection in the subject includes an active ingredient of rosuvastatin or a pharmaceutically acceptable salt thereof (e.g., rosuvastatin calcium, etc.) In some embodiments, the statin pharmaceutical composition includes an active ingredient of rosuvastatin calcium.

In some embodiments, the statin therapeutic composition for the treatment of the coronavirus infection in the subject includes rosuvastatin (CRESTOR®) as an active ingredient. In some embodiments, the statin therapeutic composition includes a composition disclosed in U.S. Pat. Nos. 6,316,460 or 6,858,618, each of which is hereby incorporated by reference, as an active ingredient. In some embodiments, the statin therapeutic composition for the treatment of the coronavirus infection in the subject includes atorvastatin (LIPITOR®) as an active ingredient. In some embodiments, the statin therapeutic composition for the treatment of the coronavirus infection in the subject includes fluvastatin (LESCOL®, LESCOL XL®) as an active ingredient. In some embodiments, the statin therapeutic composition includes a composition disclosed in U.S. Pat. No. 6,242,003, which is hereby incorporated by reference, as an active ingredient.

In some embodiments, the statin therapeutic composition for the treatment of the coronavirus infection in the subject includes lovastatin (ALTOPREV®) as an active ingredient. In some embodiments, the statin therapeutic composition for the treatment of the coronavirus infection in the subject includes pitavastatin (LIVALO®) as an active ingredient. In some embodiments, the statin therapeutic composition includes a composition disclosed in U.S. Pat. Nos. 5,856,336, 7,022,713, or 8557993, each of which is hereby incorporated by reference, as an active ingredient. In some embodiments, the statin therapeutic composition for the treatment of the coronavirus infection in the subject includes pravastatin (PRAVACHOL®) as an active ingredient. In some embodiments, the statin therapeutic composition for the treatment of the coronavirus infection in the subject includes simvastatin (ZOCOR®) as an active ingredient.

In some embodiments, the statin therapeutic composition for the treatment of the coronavirus infection in the subject includes a statin composition described in Lee et al., 2007, “Comparison of Efficacy and Tolerability of Pitavastatin and Atorvastatin: an 8-Week, Multicenter, Randomized, Open-Label, Dose-Titration Study in Korean Patients with Hypercholesterolemia,” Clin Ther. 2007; 29:2365-73; Bradford et al., 1990, “Expanded clinical evaluation of lovastatin (EXCEL) study design and patient characteristics of a double blind, placebo controlled study in patients with moderate hypercholesterolemia. American Journal of Cardiology 66: p.44B-55B; Serruys et al., 2002, “Fluvastatin for Prevention of Cardiac Events Following Successful First Percutaneous Coronary Intervention: A Randomized Controlled Trial.,” JAMA 287:p.3215-3222; Sacks et al. 1996, “The effect of pravastatin on coronary events after myocardial infarction in patients with average cholesterol levels. Cholesterol and Recurrent Events Trial investigators,” New England Journal of Medicine, 1996. 335(14): p. 001-9; Anonymous, 2002 “Heart Protection Study Collaborative Group, MRC/BHF Heart Protection Study of cholesterol lowering with simvastatin in 20,536 high-risk individuals: a randomised placebo-controlled trial,” Lancet 360: p. 7-22; Jones et al., 2003, “Comparison of the efficacy and safety of rosuvastatin versus atorvastatin, simvastatin, and pravastatin across doses (STELLAR Trial), “Am J Cardiol. 92 (2): 152-60 each of which is hereby incorporated by reference herein in its entirety.

In some embodiments, the administration of the statin is used for treatment of a disease related to a coronavirus infection in the subject. For example, in some embodiments, the disease related to the coronavirus infection is an acute, midterm or long-term onset of clinical or health complications caused by a coronavirus infection. In some embodiments, the coronavirus infection is a severe acute respiratory syndrome-related coronavirus (SARS-CoV) infection. In some embodiments, the SARS-CoV infection is a severe acute respiratory syndrome-related coronavirus 2 (SARS-CoV-2) infection. In some embodiments, the subject has been diagnosed with COVID-19. In some embodiments, the SARS-CoV infection is a severe acute respiratory syndrome-related coronavirus 1 (SARS-CoV-1) infection. In some embodiments, the subject has been diagnosed with SARS. In some embodiments, the infection is a Middle East respiratory syndrome-related coronavirus (MERS-CoV). In some embodiments, the subject has been diagnosed with MERS.

In some embodiments, the treatment of the disease related to a coronavirus infection comprises prevention of acute, midterm or long-term clinical or health complications caused by a coronavirus infection (e.g., SARS-CoV-2, SARS-CoV-1, and/or MERS-CoV). In some embodiments, the treatment comprises amelioration of symptoms of acute, midterm or long-term clinical or health complications caused by a coronavirus infection (e.g., SARS-CoV-2, SARS-CoV-1, and/or MERS-CoV). In some embodiments, the treatment comprises a cure for acute, midterm or long-term clinical or health complications caused by a coronavirus infection (e.g., SARS-CoV-2, SARS-CoV-1, and/or MERS-CoV).

In some embodiments, the statin administered for the treatment of the disease related to a coronavirus infection in the subject is rosuvastatin, atorvastatin, fluvastatin, lovastatin, pitavastatin, pravastatin, or simvastatin, and/or any combination or pharmaceutically acceptable salt thereof. In some embodiments, the statin administered for the treatment of the disease related to a coronavirus infection in the subject is co-administered with another lipid-lowering drug (e.g., ezetimibe, niacin, lomitapide, bempedoic acid, mipomersen, sebelipase, glybera, volanesorsen, evinacumab, or lecithin). For example, in some embodiments, the statin administered for the treatment of the disease related to a coronavirus infection in the subject is Atorvastatin/Ezetimibe (LIPTRUZET®), Lovastatin+Niacin (ADVICOR®), Simvastatin/Ezetimibe (VYTORIN®), or Simvastatin/Niacin-ER (SIMCOR®).

In some embodiments, the statin administered for the treatment of the disease related to a coronavirus infection in the subject is a prodrug. As used herein, a prodrug refers to a pharmaceutical composition that includes a biologically inactive compound that is metabolized in vivo to generate the active form of the drug. For instance, in some embodiments, the prodrug statin is rosuvastatin, atorvastatin, fluvastatin, lovastatin, pitavastatin, pravastatin, or simvastatin.

In some embodiments, the statin to be administered for the treatment of the disease related to a coronavirus infection in the subject is in the form of a statin therapeutic composition comprising an active ingredient (e.g., rosuvastatin, atorvastatin, fluvastatin, lovastatin, pitavastatin, pravastatin, and/or simvastatin), or a combination of active ingredients and/or a pharmaceutically acceptable salt thereof.

For example, in some embodiments, the statin therapeutic composition for the treatment of the disease related to a coronavirus infection in the subject includes an active ingredient of rosuvastatin or a pharmaceutically acceptable salt thereof (e.g., rosuvastatin calcium, etc.) In some embodiments, the statin pharmaceutical composition includes an active ingredient of rosuvastatin calcium.

In some embodiments, the statin therapeutic composition for the treatment of the disease related to a coronavirus infection in the subject includes rosuvastatin (CRESTOR®) as an active ingredient. In some embodiments, the statin therapeutic composition includes a composition disclosed in U.S. Pat. Nos. 6,316,460 or 6,858,618, each of which is hereby incorporated by reference, as an active ingredient. In some embodiments, the statin therapeutic composition for the treatment of the disease related to a coronavirus infection in the subject includes atorvastatin (LIPITOR®) as an active ingredient. In some embodiments, the statin therapeutic composition for the treatment of the disease related to a coronavirus infection in the subject includes fluvastatin (LESCOL®, LESCOL XL®) as an active ingredient. In some embodiments, the statin therapeutic composition includes a composition disclosed in U.S. Pat. No. 6,242,003, which is hereby incorporated by reference, as an active ingredient.

In some embodiments, the statin therapeutic composition for the treatment of the disease related to a coronavirus infection in the subject includes lovastatin (ALTOPREV®) as an active ingredient. In some embodiments, the statin therapeutic composition for the treatment of the disease related to a coronavirus infection in the subject includes pitavastatin (LIVALO®) as an active ingredient. In some embodiments, the statin therapeutic composition includes a composition disclosed in U.S. Pat. Nos. 5,856,336, 7,022,713, or 8557993, each of which is hereby incorporated by reference, as an active ingredient. In some embodiments, the statin therapeutic composition for the treatment of the disease related to a coronavirus infection in the subject includes pravastatin (PRAVACHOL®) as an active ingredient. In some embodiments, the statin therapeutic composition for the treatment of the disease related to a coronavirus infection in the subject includes simvastatin (ZOCOR®) as an active ingredient.

In some embodiments, the statin therapeutic composition for the treatment of the disease related to a coronavirus infection in the subject includes a statin composition described in Lee et al., 2007, “Comparison of Efficacy and Tolerability of Pitavastatin and Atorvastatin: an 8-Week, Multicenter, Randomized, Open-Label, Dose-Titration Study in Korean Patients with Hypercholesterolemia,” Clin Ther. 2007; 29:2365-73; Bradford et al., 1990, “Expanded clinical evaluation of lovastatin (EXCEL) study design and patient characteristics of a double blind, placebo controlled study in patients with moderate hypercholesterolemia. American Journal of Cardiology 66: p.44B-55B; Serruys et al., 2002, “Fluvastatin for Prevention of Cardiac Events Following Successful First Percutaneous Coronary Intervention: A Randomized Controlled Trial.,” JAMA 287:p.3215-3222; Sacks et al. 1996, “The effect of pravastatin on coronary events after myocardial infarction in patients with average cholesterol levels. Cholesterol and Recurrent Events Trial investigators,” New England Journal of Medicine, 1996. 335(14): p. 001-9; Anonymous, 2002 “Heart Protection Study Collaborative Group, MRC/BHF Heart Protection Study of cholesterol lowering with simvastatin in 20,536 high-risk individuals: a randomised placebo-controlled trial,” Lancet 360: p. 7-22; Jones et al., 2003, “Comparison of the efficacy and safety of rosuvastatin versus atorvastatin, simvastatin, and pravastatin across doses (STELLAR Trial), “Am J Cardiol. 92 (2): 152-60 each of which is hereby incorporated by reference herein in its entirety.

In some embodiments, the method further comprises co-administering a therapeutically effective amount of a Klotho polypeptide (e.g., α-Klotho, β-Klotho, and/or γ-Klotho) to the subject. In some embodiments, the method further comprises co-administering a therapeutically effective amount of an inhibitor of the mTOR pathway to the subject. In some embodiments, the method further comprises co-administering a therapeutically effective amount of an inhibitor of the NF-κB pathway to the subject. In some embodiments, the method further comprises co-administering a therapeutically effective amount of a lipid-reducing compound to the subject.

### SPECIFIC EMBODIMENTS

In one aspect, the present disclosure provides a method for treating a severe acute respiratory syndrome-related coronavirus (SARS-CoV) infection in a subject in need thereof, the method comprising administering a therapeutically effective amount of a Klotho polypeptide to the subject.

In some embodiments, the SARS-CoV infection is a severe acute respiratory syndrome-related coronavirus 2 (SARS-CoV-2) infection.

In some embodiments, the Klotho polypeptide is a recombinant Klotho polypeptide. In some embodiments, the recombinant Klotho polypeptide is modified with a water-soluble polypeptide. In some embodiments, the recombinant Klotho polypeptide is a fusion protein with a half-life extending peptide moiety.

In some embodiments, the Klotho polypeptide is purified from a pool of blood plasma or blood serum from at least 1000 donors.

In some embodiments, the Klotho polypeptide is administered by intravenous infusion.

In some embodiments, the Klotho polypeptide is administered by subcutaneous injection.

In another aspect, the present disclosure provides a method for treating a severe acute respiratory syndrome-related coronavirus (SARS-CoV) infection in a subject in need thereof, the method comprising administering a Klotho polynucleotide encoding a Klotho polypeptide to the subject.

In some embodiments, the method comprises administering to the subject a viral-based gene therapy vector comprising the Klotho polynucleotide.

In some such embodiments, the viral-based gene therapy vector is an adeno-associated viral (AAV) gene therapy vector.

In some embodiments, the Klotho polypeptide is an α-Klotho polypeptide.

In some embodiments, the α-Klotho polypeptide comprises a KL1 glycosyl hydrolase-1 domain and a KL2 glycosyl hydrolase-2 domain. In some embodiments, the α-Klotho polypeptide comprises a KL1 glycosyl hydrolase-1 domain, but not a KL2 glycosyl hydrolase-2 domain.

In some embodiments, the α-Klotho polypeptide is a human α-Klotho polypeptide.

In some embodiments, the human α-Klotho polypeptide comprises an amino acid sequence having at least 95% identity to amino acids 34-981 of SEQ ID NO:1 (the full-length, wild-type sequence of the human Klotho precursor protein—NP004786). In some embodiments, the human α-Klotho polypeptide comprises an amino acid sequence having at least 99% identity to amino acids 34-981 of SEQ ID NO:1 (the full-length, wild-type sequence of the human Klotho precursor protein—NP004786). In some embodiments, the human α-Klotho polypeptide comprises an amino acid sequence of amino acids 34-981 of SEQ ID NO:1 (the full-length, wild-type sequence of the human Klotho precursor protein—NP004786).

In some embodiments, the human α-Klotho polypeptide comprises an amino acid sequence having at least 95% identity to amino acids 34-549 of SEQ ID NO:1 (the full-length, wild-type sequence of the human Klotho precursor protein—NP004786). In some embodiments, the human α-Klotho polypeptide comprises an amino acid sequence having at least 99% identity to amino acids 34-549 of SEQ ID NO:1 (the full-length, wild-type sequence of the human Klotho precursor protein—NP004786). In some embodiments, the human α-Klotho polypeptide comprises an amino acid sequence of amino acids 34-549 of SEQ ID NO:1 (the full-length, wild-type sequence of the human Klotho precursor protein—NP004786).

In some embodiments, the human α-Klotho polypeptide comprises an amino acid sequence having at least 95% identity to amino acids 34-506 of SEQ ID NO:1 (the full-length, wild-type sequence of the human Klotho precursor protein—NP004786). In some embodiments, the human α-Klotho polypeptide comprises an amino acid sequence having at least 99% identity to amino acids 34-506 of SEQ ID NO:1 (the full-length, wild-type sequence of the human Klotho precursor protein—NP004786). In some embodiments, the human α-Klotho polypeptide comprises an amino acid sequence of amino acids 34-506 of SEQ ID NO:1 (the full-length, wild-type sequence of the human Klotho precursor protein—NP004786).

In some embodiments, the Klotho polypeptide is a β-Klotho polypeptide.

In some embodiments, the β-Klotho polypeptide comprises a KL1 glycosyl hydrolase-1 domain and a KL2 glycosyl hydrolase-2 domain. In some embodiments, the β-Klotho polypeptide comprises a KL1 glycosyl hydrolase-1 domain, but not a KL2 glycosyl hydrolase-2 domain.

In some embodiments, the β-Klotho polypeptide is a human β-Klotho polypeptide.

In some embodiments, the human β-Klotho polypeptide comprises an amino acid sequence having at least 95% identity to amino acids 54-996 of SEQ ID NO:2 (the full-length, wild-type sequence of the human β-Klotho precursor protein—NP783864). In some embodiments, the human β-Klotho polypeptide comprises an amino acid sequence having at least 99% identity to amino acids 54-996 of SEQ ID NO:2 (the full-length, wild-type sequence of the human β-Klotho precursor protein—NP783864). In some embodiments, the human β-Klotho polypeptide comprises an amino acid sequence of amino acids 54-996 of SEQ ID NO:2 (the full-length, wild-type sequence of the human β-Klotho precursor protein—NP783864).

In some embodiments, the human β-Klotho polypeptide comprises an amino acid sequence having at least 95% identity to amino acids 77-508 of SEQ ID NO:2 (the full-length, wild-type sequence of the human β-Klotho precursor protein—NP783864). In some embodiments, the human β-Klotho polypeptide comprises an amino acid sequence having at least 99% identity to amino acids 77-508 of SEQ ID NO:2 (the full-length, wild-type sequence of the human β-Klotho precursor protein—NP783864). In some embodiments, the human β-Klotho polypeptide comprises an amino acid sequence of amino acids 77-508 of SEQ ID NO:2 (the full-length, wild-type sequence of the human β-Klotho precursor protein—NP783864).

In some embodiments, the Klotho polypeptide is a γ-Klotho polypeptide.

In some embodiments, the γ-Klotho polypeptide is a human γ-Klotho polypeptide.

In some embodiments, the human γ-Klotho polypeptide comprises an amino acid sequence having at least 95% identity to amino acids 23-541 of SEQ ID NO:3 (the full-length, wild-type sequence of the human γ-Klotho precursor protein—NP_997221). In some embodiments, the human γ-Klotho polypeptide comprises an amino acid sequence having at least 99% identity to amino acids 23-541 of SEQ ID NO:3 (the full-length, wild-type sequence of the human γ-Klotho precursor protein—NP_997221). In some embodiments, the human γ-Klotho polypeptide comprises an amino acid sequence of amino acids 23-541 of SEQ ID NO:3 (the full-length, wild-type sequence of the human γ-Klotho precursor protein—NP_997221).

In some embodiments, the subject has been diagnosed with COVID-19.

In another aspect, the present disclosure provides a method for treating a severe acute respiratory syndrome-related coronavirus (SARS-CoV) infection in a subject in need thereof, the method comprising determining whether the subject has diminished Klotho activity by obtaining a blood sample from the subject, determining an amount of Klotho protein in the blood sample or a level of Klotho activity in the blood sample, and comparing the amount of Klotho protein in the blood sample or the level of Klotho activity in the blood sample to a predetermined threshold, thereby determining whether the subject has diminished Klotho activity. The method further comprises, when the subject has diminished Klotho activity, administering a first therapy for SARS-CoV infection to the subject, and when the subject does not have diminished Klotho activity, administering a second therapy for SARS-CoV infection to the subject that is different from the first therapy.

In some embodiments, the SARS-CoV infection is a severe acute respiratory syndrome-related coronavirus 2 (SARS-CoV-2) infection.

In some embodiments, the subject has been diagnosed with COVID-19.

In some embodiments, the Klotho protein is α-Klotho.

In some embodiments, the Klotho protein is β-Klotho.

In some embodiments, the Klotho protein is γ-Klotho.

In some embodiments, the first therapy comprises administering a therapeutically effective amount of a Klotho polypeptide to the subject.

In some embodiments, the Klotho polypeptide is a recombinant Klotho polypeptide. In some embodiments, the recombinant Klotho polypeptide is modified with a water-soluble polypeptide. In some embodiments, the recombinant Klotho polypeptide is a fusion protein with a half-life extending peptide moiety.

In some embodiments, the Klotho polypeptide is purified from a pool of blood plasma or blood serum from at least 1000 donors.

In some embodiments, the Klotho polypeptide is administered by intravenous infusion.

In some embodiments, the Klotho polypeptide is administered by subcutaneous injection.

In some embodiments, the first therapy comprises administering a Klotho polynucleotide encoding an Klotho polypeptide to the subject.

In some embodiments, the method comprises administering to the subject a viral-based gene therapy vector comprising the Klotho polynucleotide.

In some embodiments, the viral-based gene therapy vector is an adeno-associated viral (AAV) gene therapy vector.

In some embodiments, the Klotho polypeptide is an α-Klotho polypeptide.

In some embodiments, the α-Klotho polypeptide comprises a KL1 glycosyl hydrolase-1 domain and a KL2 glycosyl hydrolase-2 domain. In some embodiments, the α-Klotho polypeptide comprises a KL1 glycosyl hydrolase-1 domain, but not a KL2 glycosyl hydrolase-2 domain.

In some embodiments, the α-Klotho polypeptide is a human α-Klotho polypeptide.

In some embodiments, the human α-Klotho polypeptide comprises an amino acid sequence having at least 95% identity to amino acids 34-981 of SEQ ID NO:1 (the full-length, wild-type sequence of the human Klotho precursor protein—NP004786). In some embodiments, the human α-Klotho polypeptide comprises an amino acid sequence having at least 99% identity to amino acids 34-981 of SEQ ID NO:1 (the full-length, wild-type sequence of the human Klotho precursor protein—NP004786). In some embodiments, the human α-Klotho polypeptide comprises an amino acid sequence of amino acids 34-981 of SEQ ID NO:1 (the full-length, wild-type sequence of the human Klotho precursor protein—NP004786).

In some embodiments, the human α-Klotho polypeptide comprises an amino acid sequence having at least 95% identity to amino acids 34-549 of SEQ ID NO:1 (the full-length, wild-type sequence of the human Klotho precursor protein—NP004786). In some embodiments, the human α-Klotho polypeptide comprises an amino acid sequence having at least 99% identity to amino acids 34-549 of SEQ ID NO:1 (the full-length, wild-type sequence of the human Klotho precursor protein—NP004786). In some embodiments, the human α-Klotho polypeptide comprises an amino acid sequence of amino acids 34-549 of SEQ ID NO:1 (the full-length, wild-type sequence of the human Klotho precursor protein—NP004786).

In some embodiments, the human α-Klotho polypeptide comprises an amino acid sequence having at least 95% identity to amino acids 34-506 of SEQ ID NO:1 (the full-length, wild-type sequence of the human Klotho precursor protein—NP004786). In some embodiments, the human α-Klotho polypeptide comprises an amino acid sequence having at least 99% identity to amino acids 34-506 of SEQ ID NO:1 (the full-length, wild-type sequence of the human Klotho precursor protein—NP004786). In some embodiments, the human α-Klotho polypeptide comprises an amino acid sequence of amino acids 34-506 of SEQ ID NO:1 (the full-length, wild-type sequence of the human Klotho precursor protein—NP004786).

In some embodiments, the Klotho polypeptide is a β-Klotho polypeptide.

In some embodiments, the β-Klotho polypeptide comprises a KL1 glycosyl hydrolase-1 domain and a KL2 glycosyl hydrolase-2 domain. In some embodiments, the β-Klotho polypeptide comprises a KL1 glycosyl hydrolase-1 domain, but not a KL2 glycosyl hydrolase-2 domain.

In some embodiments, the β-Klotho polypeptide is a human β-Klotho polypeptide.

In some embodiments, the human β-Klotho polypeptide comprises an amino acid sequence having at least 95% identity to amino acids 54-996 of SEQ ID NO:2 (the full-length, wild-type sequence of the human β-Klotho precursor protein—NP783864). In some embodiments, the human β-Klotho polypeptide comprises an amino acid sequence having at least 99% identity to amino acids 54-996 of SEQ ID NO:2 (the full-length, wild-type sequence of the human β-Klotho precursor protein—NP783864). In some embodiments, the human β-Klotho polypeptide comprises an amino acid sequence of amino acids 54-996 of SEQ ID NO:2 (the full-length, wild-type sequence of the human β-Klotho precursor protein—NP783864).

In some embodiments, the human β-Klotho polypeptide comprises an amino acid sequence having at least 95% identity to amino acids 77-508 of SEQ ID NO:2 (the full-length, wild-type sequence of the human β-Klotho precursor protein—NP783864). In some embodiments, the human β-Klotho polypeptide comprises an amino acid sequence having at least 99% identity to amino acids 77-508 of SEQ ID NO:2 (the full-length, wild-type sequence of the human β-Klotho precursor protein—NP783864). In some embodiments, the human β-Klotho polypeptide comprises an amino acid sequence of amino acids 77-508 of SEQ ID NO:2 (the full-length, wild-type sequence of the human β-Klotho precursor protein—NP783864).

In some embodiments, the Klotho polypeptide is a γ-Klotho polypeptide.

In some embodiments, the γ-Klotho polypeptide is a human γ-Klotho polypeptide.

In some embodiments, the human γ-Klotho polypeptide comprises an amino acid sequence having at least 95% identity to amino acids 23-541 of SEQ ID NO:3 (the full-length, wild-type sequence of the human γ-Klotho precursor protein—NP_997221). In some embodiments, the human γ-Klotho polypeptide comprises an amino acid sequence having at least 99% identity to amino acids 23-541 of SEQ ID NO:3 (the full-length, wild-type sequence of the human γ-Klotho precursor protein—NP_997221). In some embodiments, the human γ-Klotho polypeptide comprises an amino acid sequence of amino acids 23-541 of SEQ ID NO:3 (the full-length, wild-type sequence of the human γ-Klotho precursor protein—NP_997221).

In another aspect, the present disclosure provides a method for treating a severe acute respiratory syndrome-related coronavirus (SARS-CoV) infection in a subject in need thereof, the method comprising administering a therapeutically effective amount of an inhibitor of the mTOR pathway.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the inhibitor of the mTOR pathway targets phosphoinositide 3-kinase (PI3K). In some embodiments, the phosphoinositide 3-kinase (PI3K) is a Class I PI3K, a Class II PI3K, a Class III PI3K, or a Class IV PI3K. In some embodiments, the catalytic subunit of the Class I PI3K is p110α, p110β, p110δ or p110γ. In some embodiments, the inhibitor is a pan-PI3K class I inhibitor. In some embodiments, the inhibitor is an isoform-specific PI3K inhibitor. In some embodiments, the inhibitor is a dual PI3K/mTOR inhibitor.

In some embodiments, the inhibitor of the mTOR pathway targets protein kinase B (PKB/AKT).

In some embodiments, the inhibitor is an AKT inhibitor.

In some embodiments, the inhibitor of the mTOR pathway targets mammalian target of rapamycin (mTOR). In some embodiments, mTOR is a component in mTOR complex 1 (mTORC1). In some embodiments, mTOR is a component in mTOR complex 2 (mTORC2). In some embodiments, the inhibitor is a rapamycin analog. In some embodiments, the inhibitor is a dual mTORC1/mTORC2 inhibitor. In some embodiments, the inhibitor is a dual PI3k/mTOR inhibitor.

In some embodiments, the inhibitor of the mTOR pathway targets a receptor tyrosine kinase (RTK).

In some embodiments, the method further comprises co-administering a therapeutically effective amount of a Klotho polypeptide to the subject.

In another aspect, the present disclosure provides a method for treating a severe acute respiratory syndrome-related coronavirus (SARS-CoV) infection in a subject in need thereof, the method comprising administering a therapeutically effective amount of an inhibitor of the NF-κB pathway.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the inhibitor of the NF-κB pathway targets a tumor necrosis factor receptor (TNF-R). In some embodiments, the inhibitor is a member of the TRAF protein family. In some embodiments, the TRAF protein is a dominant negative mutant. In some embodiments, the inhibitor is a kinase. In some embodiments, the kinase is a kinase-deficient or dominant negative mutant.

In some embodiments, the inhibitor of the NF-κB pathway targets an IκB kinase (IKK) complex. In some embodiments, the inhibitor targets IKKα. In some embodiments, the inhibitor targets IKKβ. In some embodiments, the inhibitor targets IKKγ (NEMO). In some embodiments, the inhibitor is an ATP analog. In some embodiments, the inhibitor is a thiol-reactive compound that interacts with a cysteine residue on the target TKK. In some embodiments, the inhibitor is a dominant-negative mutant of IKKα, IKKβ, or IKKγ.

In some embodiments, the inhibitor of the NF-κB pathway inhibits ubiquitination or proteasomal degradation of IκB. In some embodiments, the inhibitor is a peptide aldehyde, a cysteine protease inhibitor, a β-lactone, a dipeptidyl boronate, or a serine protease inhibitor.

In some embodiments, the inhibitor of the NF-κB pathway inhibits nuclear translocation of NF-κB. In some embodiments, the inhibitor is a cell-permeable peptide.

In some embodiments, the inhibitor of the NF-κB pathway inhibits DNA binding of NF-κB. In some embodiments, the inhibitor is a sesquiterpene lactone.

In some embodiments, the inhibitor of the NF-κB pathway inhibits transcriptional activation of NF-κB. In some embodiments, the inhibitor selectively inhibits phosphatidylcholine-phospholipase C inhibitor, protein kinase C or p38 MAPK.

In some embodiments, the inhibitor of the NF-κB pathway is an inhibitor of KB (IκB).

In some embodiments, the inhibitor of the NF-κB pathway is a protein, a peptide, an antioxidant, or a small molecule.

In some embodiments, the method further comprises co-administering a therapeutically effective amount of a Klotho polypeptide to the subject.

In another aspect, the present disclosure provides a method for treating a severe acute respiratory syndrome-related coronavirus (SARS-CoV) infection in a subject with hyperlipidemia and in need thereof, the method comprising administering a therapeutically effective amount of a lipid-reducing compound.

In some embodiments, the SARS-CoV infection is a severe acute respiratory syndrome-related coronavirus 2 (SARS-CoV-2) infection. In some embodiments, the subject has been diagnosed with COVID-19.

In some embodiments, the lipid is a low-density lipoprotein (LDL). In some embodiments, the lipid is a high-density lipoprotein (HDL). In some embodiments, the lipid is triglyceride. In some embodiments, the lipid is lipoprotein(a).

In some embodiments, the lipid-reducing compound is a statin. In some embodiments, the lipid-reducing compound is a bile acid sequestrant. In some embodiments, the lipid-reducing compound is a PCSK9 inhibitor. In some embodiments, the lipid-reducing compound is a fibrate. In some embodiments, the lipid-reducing compound is ezetimibe, niacin, lomitapide, bempedoic acid, mipomersen, sebelipase, glybera, volanesorsen, evinacumab, or lecithin. In some embodiments, the lipid-reducing compound is an HDL-based peptide.

In some embodiments, the subject was not previously treated with a lipid-reducing compound. In some embodiments, the subject was previously treated with a lipid-reducing compound, and the administering a therapeutically effective amount of the lipid-reducing compound includes increasing the dosage of the compound.

In some embodiments, the method further comprises co-administering a therapeutically effective amount of a Klotho polypeptide to the subject. In some embodiments, the method further comprises co-administering a therapeutically effective amount of an inhibitor of the mTOR pathway to the subject. In some embodiments, the method further comprises co-administering a therapeutically effective amount of an inhibitor of the NF-κB pathway to the subject.

### EXAMPLES

### Example 1—KLOTHO as a Central Agent in COVID-19 Disease

SARS-CoV-2, a novel coronavirus, has caused a global pandemic of COVID-19. This disease is characterized by diverse manifestations, ranging from asymptomatic infections to severe cases and death. Throughout the course of infection, patients can present a broad array of symptoms, including cough, fever, loss of smell, and shortness of breath, with the potential of developing severe complications such as respiratory failure, kidney injury, multi-organ failure, micro-coagulation, stroke, thrombosis, and cytokine release syndrome. Intriguingly, Kawasaki disease-like manifestations have been described to occur in children and adolescents in the context of COVID-19. Risk factors for severity in COVID-19 disease are diverse, such as advanced age, hypertension, uncontrolled diabetes mellitus, obesity, dyslipidemia, smoking, chronic kidney disease (CKD), cancer, and chronic obstructive pulmonary disease (COPD). A striking feature of COVID-19 is that the factors shown to be by far the most robustly associated with both its severity and its mortality are also risk factors for chronological and biological aging. Biomedical research has advanced understanding of the virus at an unprecedented pace. Nevertheless, the diversity of risk factors, symptoms, and health complications of COVID-19 has conventionally eluded a mechanistic explanation. The present example describes indications that Klotho, an anti-aging protein, plays a central role in COVID-19 that can explain the diversity of corresponding risk factors and clinical outcomes. Klotho is involved in numerous biological processes that share considerable overlap with known mechanisms of SARS-CoV-2 infection and clinical deterioration to severe COVID-19 cases. In some embodiments, the status of serum Klotho deficiency can underlie the pathological lung-kidney, and potentially, cardio-renal axes. In some embodiments, a central role for Klotho in COVID-19 evolution opens new avenues for research into the nature of SARS-CoV-2 infections, and perhaps, more importantly, indicates potential new treatments for health complications from infection with SARS-CoV-2 and other coronaviruses that may emerge in the future.

Infection by SARS-CoV-2 can cause a surprising diversity of clinical manifestations, ranging from a fully asymptomatic condition or mild disease (fever, cough, gastrointestinal symptoms, loss of smell), to severe cases with the potential to evolve into respiratory failure, renal injury, multi-organ failure, micro-coagulation, thrombosis, stroke, and cytokine release syndrome, as well as Kawasaki disease-like features in children and adolescents [1-3].

The identified risk factors for severe cases are equally diverse, including advanced age, hypertension, diabetes mellitus (especially uncontrolled DM), obesity, smoking, dyslipidemia, chronic kidney disease (CKD), cancer, and chronic obstructive pulmonary disease (COPD) [4, 5]. However, results from a) meta-analyses from pooled data stemming from several cohorts and b) OpenSAFELY database have clearly identified that the association of those risk factors specifically related to (premature) human aging (e.g., such as CKD) are robustly associated with severity and lethality from SARS-CoV-2 [6-11]. To a lesser extent, other aging-related diseases have also been identified as important risk factors for COVID-19 lethality, such as chronic respiratory diseases—in particular, COPD [12-14].

No unifying agent or signaling pathway has been identified as of the date of this filing that can explain the diversity of risk factors, symptoms, and clinical manifestations caused by a SARS-CoV-2 viral infection. Without being limited by any one theory of operation, in some embodiments, a mechanistic theory can jointly explain the rationale of the risk factors for severity, the evolution of COVID-19 disease, and the observed outcomes. Given the plethora of risk factors, biological processes and organs this virus can affect, in some embodiments, a mechanism of action may either target a central agent or signaling pathway that has a role in most or all of the involved processes, or target a number of different agents that collectively affect them all. For example, in some embodiments, a central agent hypothesis may be supported by evidence of a modest number of non-structural genes in SARS-CoV-2 genome [15].

A review of a) the mechanisms through which each risk factor can evolve into a severe clinical complication and b) the biochemical agents involved in the manifestations of known symptoms and clinical complications of COVID-19 disease identified Klotho, a protein that regulates aging [16], as a common factor in each process related to COVID-19. The identification of Klotho improves upon conventional knowledge as the first single unifying factor postulated for SARS-CoV-2 pathophysiology.

### Methods

The PubMed database (NLM, available online at ncbi.nlm.nih.gov/pubmed) was reviewed, with a special emphasis on results stemming from meta-analyses obtained with low levels of heterogeneity, as previously advised [62], with the purpose of improving the inference.

### Results

A review of the known mechanisms by which risk factors associated with COVID-19 disease can evolve into severe clinical complications, as well as the pathways that are involved in the symptoms and clinical outcomes of this disease, identified Klotho (e.g., Kl) as an agent consistently present in all processes. The Kl gene was discovered in 1997 in transgenic (kl/kl) mice that had this gene accidentally down regulated by an insertional mutation [16]. Kl/kl mice exhibited a syndrome that resembles human aging, including short lifespan, infertility, osteoporosis, arterial calcifications, severe hyperphosphatemia and emphysema, among other conditions. Kl encodes a homonymous protein, α-Klotho (from now onwards referred to simply as Klotho), whose hormonal activity was later shown to suppress mammalian aging [17, 18] and extend lifespan in mice that over-expressed Kl [19]. Consistent with data from animal research, serum Klotho levels have been shown to play key roles in a number of relevant biological processes in human health [20]. As highlighted below, a reduction in serum Klotho levels strongly correlates with a) the main risk factors for severity and lethality in COVID-19 (Table 3), and b) the clinical symptoms and complications in this disease (Table 4).

### Mechanistic Link Between SARS-CoV-2 and Klotho-FGF23 Axis

Similar to the previous SARS-CoV coronavirus, SARS-CoV-2 uses the angiotensin converting enzyme 2 (ACE2) as the internalization receptor to enter the cells, facilitated by the transmembrane protease serine 2 (TMPRSS2) [21]. ACE2 belongs to the canonical RAA (renin-angiotensin-aldosterone) axis and its main function is to cleave angiotensin II into angiotensin 1-7, a molecule with important vasodilatory and anti-inflammatory effects [22]. Thus, ACE2 exerts a counterbalance effect to the deleterious cardiovascular consequences of excess angiotensin II and aldosterone [22, 23]. There does not seem to be an association between ACE2 activity and SARS-CoV-2 infectivity [21]. Consistently, the data from meta-analysis have shown a neutral effect of RAA inhibitors [24], although sub-group analysis has shown important differences across ethnicities, especially for patients from Asian ancestry [24].

The joint expression of ACE2 and TMPRSS2 is important for viral tropism [25]. The continuous formation of the complex composed of SARS-CoV-2 Spike protein and ACE2 leads to ACE2 depletion [23, 25], therefore inducing a detrimental clinical outcome due to the loss of the beneficial effect of ACE2 in generating angiotensin 1-7 (antioxidant, anti-inflammatory and vasodilatory effects) [26].

An important cross talk between the RAA and Klotho-FGF23 axes has been described [27, 28]. Through non-canonical pathways (FGFR4-PLCT), excess FGF23 hyperactivates the RAA axis and downregulates Ace2, inducing adverse effects such as myocardial hypertrophy and fibrosis [29, 30]. Both a) the hyperactivation of RAA axis [27, 28, 31] and excess FGF23 [30, 32] downregulate the renal expression of KL, contributing to the adverse effects of angiotensin II and aldosterone excess.

Several consequences of Kl downregulation are explained by resistance to the FGF23 phosphaturic actions and the induction of the non-canonical FGFR4 pathway, especially in the heart, liver, and neutrophils, with adverse consequences such as left ventricular hypertrophy, increased synthesis of inflammatory mediators and impaired neutrophil recruitment, respectively [33].

Notably, SARS-CoV-2 has shown a deep tropism for kidney tissue [34]. Robust cumulative evidence has identified kidney involvement as highly deleterious for COVID-19 clinical evolution, both a) as a risk factor (chronic kidney disease, CKD) and b) as an acute complication (acute kidney injury, AKI) [6, 7, 35, 36]. Both CKD and AKI induce an upregulation of FGF23 levels and downregulation of Klotho levels; AKI does so strikingly [32].

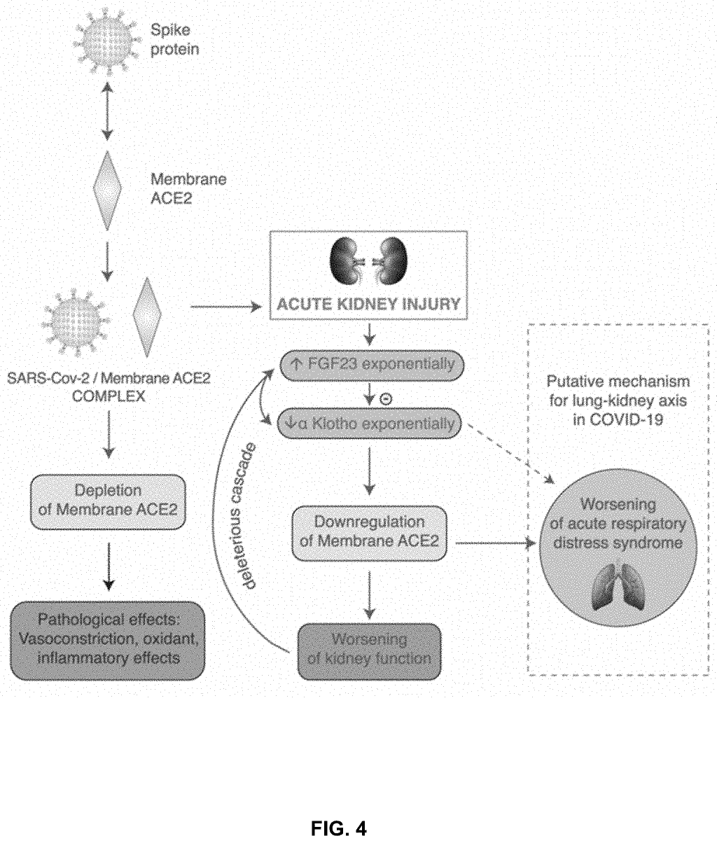

In this context, ACE2 depletion induced by SARS-CoV-2 is further aggravated by excess FGF23, as this phosphatonin induces Ace2 downregulation [29, 30, 37]. Some common diseases that have been identified as risk factors for severe COVID-19 cases are characterized by ACE2 depletion as an important pathological mechanism (e.g. CKD in the context of diabetes mellitus) [38]. Importantly, ACE2 depletion worsens not only kidney function [39, 40] but also acute respiratory distress syndrome [21,41]. Furthermore, AKI induced by SARS-CoV-2 may generate a deleterious cascade, as illustrated in FIG. 4.

A recent publication proposed a new hypothesis involving bradykinin storm as a central mechanism for COVID-19 physiopathology [42]. The research was carried out on gene expression data from bronchoalveolar lavage fluid and KL is not normally expressed in lung tissue [43]. Klotho has been reported to be critical for lung health and alveolar integrity, but these actions are mediated by soluble Klotho through its hormonal effects [43].

The role of serum FGF23 and phosphate levels in severity and mortality from COVID-19 remains to be investigated, especially in the context of AKI. Both FGF23 [32] and increased phosphate levels (and phosphate intake) [44] downregulates renal Kl expression and are potential inductors of damage not only at the kidney level, but also in myocardium and lung tissue [45-47]. Increased serum phosphate levels, even within normal ranges, are associated with mortality and worsening of kidney function; remarkably, these results have only been found in men [46, 48].

Klotho has been proposed as a strong candidate to underlie the lung-kidney axis [49], postulated recently of high relevance in severe COVID-19 [50].

### DISCUSSION

Without being limited by any one theory of operation, the above findings are consistent with the placement of the Klotho signaling pathway at the center of a unified mechanism that explains the risk factors, complications and evolution of COVID-19 disease since abnormally low serum Klotho levels correlate strongly with known symptoms and clinical complications from this disease. As such, the present disclosure provides methods comprising direct and/or indirect mechanism of down regulation of Klotho expression by SARS-CoV-2. The Klotho premise is consistent with the rarity of severe COVID-19 cases in children and increasing frequency with age, given the higher serum Klotho levels in children and decreasing levels with advancing age [51]. The role of Klotho in other health syndromes and complications from COVID-19 are provided, such as those identified with an asterisk in Tables 3 and 4.

Accordingly, the present disclosure further provides therapeutic agents known to increase Kl expression levels [52], which in some embodiments provide opportunities for evaluation of their clinical utility in COVID-19 cases. For example, inhibitors of mTOR (mammalian Target of Rapamycin), a complex that down regulates Kl expression, are being clinically investigated as possible modulators of the severity of COVID-19 disease [53]. Metformin, another mTOR inhibitor, has been clinically tested as a potential booster of the immune response to flu vaccines, especially in the older adults, and will be tested soon in COVID-19 [54]. This interventional approach is consistent with the Klotho premise since mTOR inhibitors prevent the down regulation of Kl expression levels. In some embodiments, the presently disclosed compositions and methods further comprise the treatment of a broader spectrum of viral infections, as treatment success with an mTOR inhibitor was reported for patients with severe H1N1 pneumonia [55]. A recent meta-analysis has shown a large overlap between risk factors for mortality among SARS-CoV-2, SARS and MERS (age and chronic lung disease), suggesting that the potential role of Klotho may not be restricted to SARS-CoV-2, but could extend beyond to include other coronaviruses [56].

The repurposing of drugs with known anti-aging properties is of increasing research interest as possible COVID-19 therapeutics [57]. Additional drug candidates include other inhibitors of signaling pathways that also induce Klotho downregulation, such as NF-1<0 and ERK [58]. However, in states of acute Klotho deficiency, such as in acute kidney injury, the underlying insult and inflammation may prove these approaches to be futile. The possible resistance to experimental Kl upregulating drugs mandates the clinical evaluation of direct Klotho-replacement therapy [32].

Many viral infections induce health complications well beyond their acute phase [59]. Therefore, long term follow-up of COVID-19 patients is warranted to identify potential sequelae of SARS-CoV-2 infections, especially given the important role Klotho plays in tumor suppression, central nervous system immune system and bone mineral density [60, 61].

In conclusion, the data is abundant and consistent to support, in some embodiments, a central role of Klotho (Klotho-FGF23 axis) as a unifying agent to explain the risk factors and clinical outcomes in COVID-19 disease. This premise raises the prospect of potential pleiotropic health benefits from direct interventions that normalize serum Klotho levels in patients.

TABLE 3
Correlation between Klotho levels or expression and
risk factors for severity and lethality in COVID-19

| Risk factor | Role or correlation of Klotho | Ref. |
|---|---|---|
| Advanced age | Low Klotho is associated with lower longevity | [4, 51, 63] |
| | Serum Klotho decreases exponentially with aging | |
| | Aging is the strongest risk factor in COVID-19 | |
| Chronic kidney disease | Main cause of systemic pan-Klotho deficiency | [6, 7, 8, |
| (CKD) | Potent risk factor for COVID-19 mortality | 18, 64] |
| | Klotho deficiency worsens CKD progression and | |
| | induces the premature aging phenotype of CKD | |
| Chronic obstructive | COPD: a status of local deficiency of Klotho | [43, |
| pulmonary disease | Klotho keeps alveolar integrity during postnatal life | 65-67] |
| (COPD) | Klotho is decreased in lungs of COPD patients* | |
| Hypertension | Klotho plays an important role in the pathogenesis | [4, 68] |
| | of hypertension in the elderly | |
| Diabetes mellitus (DM) | KL is downregulated in type 2 DM | [69, 70] |
| Obesity | A decrease of Klotho in obesity may partly | [71, 72] |
| | underlie its pathophysiology | |
| Smoking | Nicotine exposure downregulates Kl expression | [73, 74] |
| | and decreases glomerular filtration rate | |
| Cancer | Klotho has been identified as a tumor suppressor | [4, 75] |
| | In breast cancer partly through modulation of IGF-1 | |
| | New evidence for a role of Klotho in other cancers | |
| Dyslipidemia | Hyperlipidemia down regulates Kl expression | [5, 57, 76] |
| | in several animal models | |
| High ferritin levels | Consistent risk factor for COVID-19 severity | [77, 78] |
| | Strong negative association between serum Klotho | |
| | and ferritin levels | |
| Anorexia** | A decrease of Klotho in restricting-type anorexia | [71] |
| | nervosa may underlie its pathophysiology | |

*Klotho is nor normally expressed in lung tissue but is derived from circulation [43]
**Not yet recognized as a risk factor

TABLE 4
Correlation between Klotho levels or expression and
clinical symptoms and complications in COVID-19

| Symptom/complication | Role or correlation of Klotho | Ref. |
|---|---|---|
| Acute kidney injury | AKI induces a dramatic fall in systemic Klotho | [6, 7, 31, |
| (AKI) | levels and a statis of pan-Klotho deficiency | 34, 79] |
| | AKI is probably the most dangerous complication for | |
| | lethality in COVID-19 | |
| | The viral tropism for kidney is 100-fold greater than | |
| | for lung tissue | |
| Acute respiratory | AKI has a strong temporal association with | [31, 49, 50, |
| distress | respiratory failure and mechanical ventilation | 80, 81] |
| syndrome (lung-kidney | Klotho therapy alleviates lug injury induced by | |
| axis) | kidney injury | |
| | Therefore, Klotho is a strong candidate to underlie | |
| | the lung-kidney axis in COVID-19 | |
| Acute cardiac injury | Severe set of complications that increase mortality | [82, 83] |
| (cardiorenal axis) | substantially. | |
| | Kl expression in the heart is downregulated after | |
| | ischemic acute kidney injury | |
| Hypoxia | Kl expression is decreased under hypoxia | [84] |
| Emphysema* | Klotho is required for alveolar integrity in postnatal | [16, 67, 85] |
| | life | |
| | Kl null mice manifest severe emphysema | |
| | Autopsy series have found emphysema in COVID-19 | |
| Microcoagulation/ | PAI-1 levels, a key molecule in thrombosis, are | [86, 87] |
| thrombosis | strikingly elevated in Kl deficient mice. | |
| | In addition, PAI-1 contributes to the aging phenotype | |
| | as an important mediator of senescence | |
| Stroke | Klotho levels correlate negatively with the burden | [88, 89] |
| | and progression of cerebral vascular disease | |
| Cytokine release | IL-6 plays a key role in cytokine release syndrome | [90, 91] |
| syndrome | Klotho downregulates endothelial Il6 expression | |
| | In addition, Klotho alleviates inflammation via | |
| | modulation of Wnt1/pCREB pathway | |
| Kawasaki disease-like | Trend of Klotho levels to be decreased in children | [92, 93] |
| syndrome | with KD-like syndrome vs. healthy children. | |
| | FGF23 polymorphisms are related to cardiac | |
| | abnormalities | |
| Cognitive disorder | Cognitive impairment is associated with | [59, 94, 95] |
| | inflammation | |
| | Klotho depletion in the choroid plexus induces | |
| | inflammation and immune-mediated | |
| | neuropathogenesis | |
| | Kl overexpression enhances cognition | |
| | Kl depletion impairs memory | |
| Pre-eclampsia* | KL placental expression is decreased in pre-eclamptic | [96, 97] |
| | pregnancies. Some small studies have described pre- | |
| | eclampsia in severe COVID-19 | |
| Multi-organ failure | Sepsis creates a state of Klotho deficiency in ICU | [98-100] |
| | patients, especially in the context of AKI. | |
| | Klotho correlates with major adverse kidney events | |
| | in humans and Klotho therapy alleviates organ | |
| | damage and inflammation in rodent models with | |
| | endotoxemia | |

*Not yet recognized as clinical complications from COVID-19

### REFERENCES

1. Wiersinga, W. J.; Rhodes, A; Cheng, A. C.; Peacock, S. J. & Prescott, H. C.

Pathophysiology, Transmission, Diagnosis, and Treatment of Coronavirus Disease 2019 (COVID-19): A Review. JAMA 324(8), 782-793 (2020).
2. Yang, L. & Tu, L. Implications of gastrointestinal manifestations of COVID-19. Lancet Gastroenterol Hepatol 5(7), 629-630 (2020).
3. Toubiana, J. et al. Kawasaki-like multisystem inflammatory syndrome in children during the covid-19 pandemic in Paris, France: prospective observational study. BMJ 369, m2094 (2020).
4. Parohan, M. et al. Risk factors for mortality in patients with Coronavirus disease 2019 (COVID-19) infection: a systematic review and meta-analysis of observational studies. Aging Male, 1-9 (2020).
5. Hariyanto, T. I. & Kurniawan, A. Dyslipidemia is associated with severe coronavirus disease 2019 (COVID-19) infection. Diabetes Metab Syndr 14(5), 1463-1465 (2020).
6. Robbins-Juarez, S. Y. et al. Outcomes for Patients With COVID-19 and Acute Kidney Injury: A Systematic Review and Meta-Analysis. Kidney Int Rep 5(8), 1149-1160 (2020).
7. Gansevoort, R. T. & Hilbrands, L. B. CKD is a key risk factor for COVID-19 mortality. Nat Rev Nephrol https://doi.org/10.1038/s41581-020-00349-4 (2020).
8. Williamson, E. J. et al. Factors associated with COVID-19-related death using OpenSAFELY. Nature 584 (7821), 430-436 (2020).
9. Kooman, J. P.; Kotanko, P.; Schols, A. M. W.; Shiels, P. G. & Stenvinkel, P. Chronic kidney disease and premature ageing. Nat Rev Nephrol 10(12), 732-42 (2014).
10. Stenvinkel, P. & Larsson, T. E. Chronic kidney disease: a clinical model of premature aging. Am J Kidney Dis 62(2), 339-51 (2013).
11. Liu, Y. et al. The Chronic Kidney Disease and Acute Kidney Injury Involvement in COVID-19 Pandemic: A Systematic Review and Meta-analysis. doi: https://doi.org/10.1101/2020.04.28.20083113 (2020).
12. Algahtani, J. S. et al. Prevalence, Severity and Mortality associated with COPD and Smoking in patients with COVID-19: A Rapid Systematic Review and Meta-Analysis. PLoS One 15(5): p. e0233147 (2020).
13. Mercado, N.; Ito, K. & Barnes, P. J. Accelerated ageing of the lung in COPD: new concepts. Thorax 70(5), 482-489 (2015).
14. Kooman, J. P.; Shiels, P. G. & Stenvinkel, P. Premature aging in chronic kidney disease and chronic obstructive pulmonary disease: similarities and differences. Curr Opin Clin Nutr Metab Care 18(6), 528-34 (2015).
15. Chan, J. F. et al. Genomic characterization of the 2019 novel human-pathogenic coronavirus isolated from a patient with atypical pneumonia after visiting Wuhan. Emerg Microbes Infect 9(1), 221-236 (2020).
16. Kuro-o, M. et al. Mutation of the mouse klotho gene leads to a syndrome resembling ageing. Nature 390(6655), 45-51 (1997).
17. Ohnishi, M. & Razzaque, M. S. Dietary and genetic evidence for phosphate toxicity accelerating mammalian aging. FASEB J 24(9), 3562-3571 (2010).
18. Hu, M. C.; Shiizaki, K; Kuro-o, M. & Moe, O. W. Fibroblast growth factor 23 and Klotho: physiology and pathophysiology of an endocrine network of mineral metabolism. Annu Rev Physiol 75, 503-33 (2013).
19. Kurosu, H. et al. Suppression of aging in mice by the hormone Klotho. Science 309(5742), 1829-1833 (2005).
20. Kuro, O. M. The Klotho proteins in health and disease. Nat Rev Nephrol 15(1), 27-44 (2019).
21. Alhenc-Gelas, F. & Drueke, T. B. Blockade of SARS-CoV-2 infection by recombinant soluble ACE2. Kidney Int 97(6), 1091-1093 (2020).
22. Paz Ocaranza, M. et al. Counter-regulatory renin-angiotensin system in cardiovascular disease. Nat Rev Cardiol 17(2), 116-129 (2020).
23. Gheblawi, M. et al. Angiotensin-Converting Enzyme 2: SARS-CoV-2 Receptor and Regulator of the Renin-Angiotensin System: Celebrating the 20th Anniversary of the Discovery of ACE2. Circ Res 126(10), 1456-1474 (2020).
24. Patoulias, D. et al. Renin-Angiotensin System Inhibitors and COVID-19: a Systematic Review and Meta-Analysis. Evidence for Significant Geographical Disparities. Curr Hypertens Rep 22(11), 90 (2020).
25. Liu, P. P.; Blet, A; Smyth, D. & Li, H. The Science Underlying COVID-19: Implications for the Cardiovascular System. Circulation 142(1), 68-78 (2020).
26. de Wit, E.; van Doremalen N.; Falzarano, D. & Munster, V. J. SARS and MERS: recent insights into emerging coronaviruses. Nat Rev Microbiol 14(8), 523-34 (2016).
27. Mitani, H. et al. In vivo klotho gene transfer ameliorates angiotensin II-induced renal damage. Hypertension 39(4), 838-43 (2002).
28. Saito, K.; Ishizaka, N.; Mitani, H.; Ohno, M. & Nagai, R. Iron chelation and a free radical scavenger suppress angiotensin II-induced downregulation of klotho, an anti-aging gene, in rat. FEBS Lett 551(1-3), 58-62 (2003).
29. Pi, M. et al. Cardiovascular Interactions between Fibroblast Growth Factor-23 and Angiotensin II. Sci Rep 8(1), 12398 https://doi.org/10.1038/s41598-018-30098-1 (2018).
30. Dai, B. et al. A comparative transcriptome analysis identifying FGF23 regulated genes in the kidney of a mouse CKD model. PLoS One 7(9): e44161 (2012).
31. de Borst, M. H.; Vervloet, M. G.; ter Wee, P. M. & Navis, G. Cross talk between the renin-angiotensin-aldosterone system and vitamin D-FGF-23-klotho in chronic kidney disease. J Am Soc Nephrol 22(9), 1603-1609 (2011).
32. Christov, M.; Neyra, J. A.; Gupta, S. & Leaf, D. E. Fibroblast Growth Factor 23 and Klotho in AKI. Semin Nephrol 39(1), 57-75 (2019).
33. Vervloet, M. Renal and extrarenal effects of fibroblast growth factor 23. Nat Rev Nephrol 15(2), 109-120 (2019).
34. Puelles, V. G. et al. Multiorgan and Renal Tropism of SARS-CoV-2. N Engl J Med 383(6), 590-592 (2020).
35. Ronco, C. & Reis, T. Kidney involvement in COVID-19 and rationale for extracorporeal therapies. Nat Rev Nephrol 16(6), 308-310 (2020).
36. Gabarre, P. et al. Acute kidney injury in critically ill patients with COVID-19. Intensive Care Med 46(7):1339-1348 (2020).
37. Martin, A.; David, V. & Quarles, L. D. Regulation and function of the FGF23/klotho endocrine pathways. Physiol Rev 92(1), 131-55 (2012).
38. Nadarajah, R. et al. Podocyte-specific overexpression of human angiotensin-converting enzyme 2 attenuates diabetic nephropathy in mice. Kidney Int 82(3), 292-303 (2012).
39. Yang, X. H. et al. Role of angiotensin-converting enzyme (ACE and ACE2) imbalance on tourniquet-induced remote kidney injury in a mouse hindlimb ischemia-reperfusion model. Peptides 36(1), 60-70 (2012).
40. Santos, R. A. S. et al. The ACE2/Angiotensin-(1-7)/MAS Axis of the Renin-Angiotensin System: Focus on Angiotensin-(1-7). Physiol Rev 98(1), 505-553 (2018)
41. Essig, M.; Matt, M. & Massy, Z. The COVID-19 outbreak and the angiotensin-converting enzyme 2: too little or too much? Nephrol Dial Transplant 35(6), 1073-1075 (2020).
42. Garvin, M. R. et al. A mechanistic model and therapeutic interventions for COVID-19 involving a RAS-mediated bradykinin storm. eLife 9: e59177 doi:10.7554/eLife.59177 (2020).
43. Zhang, J. et al. Alpha-Klotho, a critical protein for lung health, is not expressed in normal lung. FASEB Bioadv 1(11), 675-687 (2019).
44. Morishita, K. et al. The progression of aging in klotho mutant mice can be modified by dietary phosphorus and zinc. J Nutr 131(12), 3182-3188 (2001).
45. Faul, C. et al. FGF23 induces left ventricular hypertrophy. J Clin Invest 121(11), 4393-4408 (2011).
46. Bai, W.; Li, J. & Liu, J. Serum phosphorus, cardiovascular and all-cause mortality in the general population: A meta-analysis. Clin Chim Acta 461, 76-82 (2016).
47. Chang, A. R.; Lazo, M.; Appel, L. J.; Gutierrez, O. M. & Grams, M. E. High dietary phosphorus intake is associated with all-cause mortality: results from NHANES III. Am J Clin Nutr 99(2), 320-327 (2014).
48. Bellasi, A. et al. Chronic kidney disease progression and outcome according to serum phosphorus in mild-to-moderate kidney dysfunction. Clin J Am Soc Nephrol 6(4), 883-891 (2011).
49. Hsia, C. C. W.; Ravikumar, P. & Ye, J. Acute lung injury complicating acute kidney injury: A model of endogenous alphaKlotho deficiency and distant organ dysfunction. Bone 100, 100-109 (2017).
50. Nangaku, M. Lung-kidney interactions in critically ill patients: consensus report of the Acute Disease Quality Initiative (ADQI) 21 Workgroup. Kidney Int 98(1), 42-44 doi: 10.1016/j.kint.2020.05.009 (2020).
51. Yamazaki, Y. et al. Establishment of sandwich ELISA for soluble alpha-Klotho measurement: Age-dependent change of soluble alpha-Klotho levels in healthy subjects.

Biochem Biophys Res Commun 398(3), 513-8 (2010).
52. Torres, P-U. et al. Klotho: an antiaging protein involved in mineral and vitamin D metabolism. Kidney Int 71(8), 730-737 (2007).
53. Zheng, Y.; Li, R. & Liu, S. Immunoregulation with mTOR inhibitors to prevent COVID-19 severity: A novel intervention strategy beyond vaccines and specific antiviral medicines. J Med Virol https://doi.org/10.1002/jmv.26009 (2020).
54. Nature 586, 352-354 doi: 10.1038/d41586-020-02856-7 (2020)
55. Wang, C. H. et al. Adjuvant treatment with a mammalian target of rapamycin inhibitor, sirolimus, and steroids improves outcomes in patients with severe H1N1 pneumonia and acute respiratory failure. Crit Care Med 42(2), 313-321 (2014).
56. Lu, L. et al. A comparison of mortality-related risk factors of COVID-19, SARS, and MERS: A systematic review and meta-analysis. J Infect 81(4), e18-e25 (2020).
57. Zhou, Y. et al. Network-based drug repurposing for novel coronavirus 2019-nCoV/SARS-CoV-2. Cell Discov 6, 14 https://doi.org/10.1038/s41421-020-0153-3 (2020).
58. Sastre, C. et al. Hyperlipidemia-associated renal damage decreases Klotho expression in kidneys from ApoE knockout mice. PLoS One 8(12), e83713 https://doi.org/10.1371/journal.pone.0083713 (2013).
59. Zhang, P. et al. Long-term bone and lung consequences associated with hospital-acquired severe acute respiratory syndrome: a 15-year follow-up from a prospective cohort study. Bone Res 8, 8 https://doi.org/10.1038/s41413-020-0084-5 (2020).
60. Zhu, L. et al. Klotho controls the brain-immune system interface in the choroid plexus. Proc Natl Acad Sci USA 115(48), E11388-E11396 (2018).
61. Komaba, H. et al. Klotho expression in osteocytes regulates bone metabolism and controls bone formation. Kidney Int 92(3), 599-611 (2017).
62. Higgins, J. P.; Thompson, S. G.; Deeks, J. J. & Altman, D. G. Measuring inconsistency in meta-analyses. BMJ 327(7414), 557-560 (2003).
63. Semba, R. D. et al. Plasma klotho and mortality risk in older community-dwelling adults. J Gerontol A Biol Sci Med Sci 66(7), 794-800 (2011).
64. Hu, M. C.; Kuro-o, M. & Moe, O. W. Klotho and chronic kidney disease. Contrib Nephrol 180, 47-63 (2013).
65. Gao, W. et al. Klotho expression is reduced in COPD airway epithelial cells: effects on inflammation and oxidant injury. Clin Sci (Lond) 129(12), 1011-1023 (2015).
66. Ravikumar, P. et al. alpha-Klotho protects against oxidative damage in pulmonary epithelia. Am J Physiol Lung Cell Mol Physiol 307(7), L566-75 (2014).
67. Suga, T. et al. Disruption of the klotho gene causes pulmonary emphysema in mice. Defect in maintenance of pulmonary integrity during postnatal life. Am J Respir Cell Mol Biol 22(1), 26-33 (2000).
68. Su, X. M. & Yang, W. Klotho protein lowered in elderly hypertension. Int J Clin Exp Med 7(8), 2347-2350 (2014).
69. Nie, F. et al. Serum klotho protein levels and their correlations with the progression of type 2 diabetes mellitus. J Diabetes Complications 31(3), 594-598 (2017).
70. Lin, Y. & Sun, Z. In vivo pancreatic beta-cell-specific expression of antiaging gene Klotho: a novel approach for preserving beta-cells in type 2 diabetes. Diabetes 64(4), 1444-1458 (2015).
71. Amitani, M. et al. Plasma klotho levels decrease in both anorexia nervosa and obesity. Nutrition 29(9), 1106-1109 (2013).
72. Popkin, B. M. et al. Individuals with obesity and COVID-19: A global perspective on the epidemiology and biological relationships. Obes Rev https://doi.org/10.1111/obr.13128 (2020).
73. Coelho, F. O. et al. Chronic nicotine exposure reduces klotho expression and triggers different renal and hemodynamic responses in klotho-haploinsufficient mice. Am J Physiol Renal Physiol 314(5), F992-F998 (2018).
74. Grundy, E. J. et al. Smoking, SARS-CoV-2 and COVID-19: A review of reviews considering implications for public health policy and practice. Tob Induc Dis 18, 58 (2020).
75. Xie, B. et al. Klotho acts as a tumor suppressor in cancers. Pathol Oncol Res 19(4), 611-617 (2013).
76. Nagai, R. et al. Endothelial dysfunction in the klotho mouse and downregulation of klotho gene expression in various animal models of vascular and metabolic diseases. Cell Mol Life Sci 57(5), 738-746 (2000).
77. Zeng, F. et al. Association of inflammatory markers with the severity of COVID-19: A meta-analysis. Int J Infect Dis 96, 467-474 (2020).
78. Xu, Y.; Peng, H. & Ke, B. α-klotho and anemia in patients with chronic kidney disease patients: A new perspective. Exp Ther Med 14(6), 5691-5695 (2017).
79. Zhou, L.; Li, Y.; Zhou, D.; Tan, R. J. & Liu, Y. Loss of Klotho contributes to kidney injury by derepression of Wnt/beta-catenin signaling. J Am Soc Nephrol 24(5), 771-785 (2013).
80. Hirsch, J. S. et al. On behalf of the Northwell COVID-19 Research Consortium and the Northwell Nephrology COVID-19 Research Consortium. Acute kidney injury in patients hospitalized with COVID-19. Kidney Int 98(1), 209-218 (2020).
81. Ravikumar, P. et al. aKlotho deficiency in acute kidney injury contributes to lung damage. J Appl Physiol (1985) 120(7), 723-732 (2016).
82. Zou, F.; Qian, Z.; Wang, Y.; Zhao, Y. & Bai, Y. Cardiac Injury and COVID-19: A Systematic Review and Meta-analysis. CJC Open 2(5), 386-394 (2020).
83. da Cruz Junho, C.; Caio-Silva, W.; Ruiz-Hurtado, G. & Carneiro-Ramos, M. S. Characterization of Klotho/FGF23 signaling in cardiorenal syndrome-induced cardiac hypertrophy. The FASEB Journal 33 https://doi.org/10.1096/fasebj. 2019.33.1_supplement.831.1 (2019).
84. Hiyama, A.; Arai, F.; Sakai, D.; Yokoyama, K. & Mochida, J. The effects of oxygen tension and antiaging factor Klotho on Wnt signaling in nucleus pulposus cells. Arthritis Res Ther 14(3): p. R105 (2012).
85. Edler, C. et al. Dying with SARS-CoV-2 infection-an autopsy study of the first consecutive 80 cases in Hamburg, Germany. Int J Legal Med 134(4): 1275-1284 (2020).
86. Eren, M. et al. PAI-1-regulated extracellular proteolysis governs senescence and survival in Klotho mice. Proc Natl Acad Sci USA 111(19), 7090-7095 (2014).
87. Takeshita, K. et al. Increased expression of plasminogen activator inhibitor-1 with fibrin deposition in a murine model of aging, “Klotho” mouse. Semin Thromb Hemost 28(6), 545-554 (2002).
88. Woo, H. G.; Chang, Y.; Ryu, D-R.; & Song, T-J. Plasma Klotho concentration is associated with the presence, burden and progression of cerebral small vessel disease in patients with acute ischaemic stroke. PLoS One 14(8): p. e0220796 (2019).
89. Lee, J. B. et al. Plasma Klotho concentrations predict functional outcome at three months after acute ischemic stroke patients. Ann Med 51(3-4), 262-269 (2019).
90. Moore, J. B. & June, C. H. Cytokine release syndrome in severe COVID-19. Science 368(6490), 473-474 (2020).
91. Shimizu, H. et al. Indoxyl sulfate downregulates renal expression of Klotho through production of ROS and activation of nuclear factor-kB. Am J Nephrol 33(4): p. 319-24 (2011).
92. Falcini, F. et al. Circulating Levels of Klotho in Kawasaki Disease: A Possible New Marker of Vascular Damage? ACR/ARHP Scientific Meeting 11 (2011).
93. Falcini, F. et al. Fibroblast growth factor 23 (FGF23) gene polymorphism in children with Kawasaki syndrome (KS) and susceptibility to cardiac abnormalities. Ital J Pediatr 39, 69 (2013).
94. Ellul, M. A. et al. Neurological associations of COVID-19. Lancet Neurol 19(9), 767-783 (2020).
95. Dubal, D. B. et al. Life extension factor klotho enhances cognition. Cell Rep 7(4), 1065-1076 (2014).
96. Giannubilo, S. R. et al. Placental klotho protein in preeclampsia: A possible link to long term outcomes. Pregnancy Hypertension: An International Journal of Women's Cardiovascular Health 2(3), 260-261 (2012).
97. Allotey, J. et al. Clinical manifestations, risk factors, and maternal and perinatal outcomes of coronavirus disease 2019 in pregnancy: living systematic review and meta-analysis. BMJ 370, m3320 (2020).
98. Jorge, L. B. et al. Klotho deficiency aggravates sepsis-related multiple organ dysfunction. Am J Physiol Renal Physiol 316(3), F438-F448 (2019).
99. Jou-Valencia, D. et al. Renal Klotho is Reduced in Septic Patients and Pretreatment With Recombinant Klotho Attenuates Organ Injury in Lipopolysaccharide-Challenged Mice. Crit Care Med 46(12), e1196-e1203 (2018).
100. Neyra, J. A. et al. Urine Klotho Is Lower in Critically Ill Patients With Versus Without Acute Kidney Injury and Associates With Major Adverse Kidney Events. Crit Care Explor 1(6), e0016 (2019).

### CONCLUSION

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for treating a severe acute respiratory syndrome-related coronavirus (SARS-CoV) infection in a subject in need thereof, the method comprising administering a therapeutically effective amount of a Klotho polypeptide to the subject.

2. The method of claim 1, wherein the SARS-CoV infection is a severe acute respiratory syndrome-related coronavirus 2 (SARS-CoV-2) infection.

3. The method of claim 1, wherein the Klotho polypeptide is a recombinant Klotho polypeptide.

4. The method of claim 3, wherein the recombinant Klotho polypeptide is modified with a water-soluble polypeptide.

5. The method of claim 3, wherein the recombinant Klotho polypeptide is a fusion protein with a half-life extending peptide moiety.

6. The method of claim 1, wherein the Klotho polypeptide is purified from a pool of blood plasma or blood serum from at least 1000 donors.

7. The method of claim 1, wherein the Klotho polypeptide is administered by intravenous infusion.

8. The method of claim 1, wherein the Klotho polypeptide is administered by subcutaneous injection.

9. (canceled)

10-11. (canceled)

12. The method of claim 1, wherein the Klotho polypeptide is an α-Klotho polypeptide.

13. The method of claim 12, wherein the α-Klotho polypeptide comprises a KL1 glycosyl hydrolase-1 domain and a KL2 glycosyl hydrolase-2 domain.

14. The method of claim 12, wherein the α-Klotho polypeptide comprises a KL1 glycosyl hydrolase-1 domain, but not a KL2 glycosyl hydrolase-2 domain.

15. The method of claim 12, wherein the α-Klotho polypeptide is a human α-Klotho polypeptide.

16. The method of claim 15, wherein the human α-Klotho polypeptide comprises an amino acid sequence having at least 95% identity to amino acids 34-981 of SEQ ID NO:1.

17-18. (canceled)

19. The method of claim 15, wherein the human α-Klotho polypeptide comprises an amino acid sequence having at least 95% identity to amino acids 34-549 of SEQ ID NO:1.

20-21. (canceled)

22. The method of claim 15, wherein the human α-Klotho polypeptide comprises an amino acid sequence having at least 95% identity to amino acids 34-506 of SEQ ID NO:1.

23-24. (canceled)

25. The method of claim 1, wherein the Klotho polypeptide is a β Klotho polypeptide.

26-34. (canceled)

35. The method of claim 1, wherein the Klotho polypeptide is a γ Klotho polypeptide.

36-39. (canceled)

40. The method of claim 1, wherein the subject has been diagnosed with COVID-19.

41-84. (canceled)

85. (canceled)

86-147. (canceled)


SEQUENCE LISTING (5 sequences)

SEQ ID NO: 1 - protein, 1012 aa, Artificial Sequence
  synthetic sequence of sp_Q9UEF7_KLOT_HUMAN Klotho OS=Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 2 - protein, 1044 aa, Artificial Sequence
  synthetic sequence of sp_Q86Z14_KLOTB_HUMAN Beta-klotho OS=Homo sapiens
MKPGCAAGSPGNEWIFFSTDEITTRYRNTMSNGGLQRSVILSALILLRAVTGFSGDGRAIWSKNPNFTPVNESQLFLYDTFPKNFFWGIGTGALQVEGSWKKDGKGPSIWDHFIHTHLKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIHNPYLVAWHGYGTGMHAPGEKGNLAAVYTVGHNLIKAHSKVWHNYNTHFRPHQKGWLSITLGSHWIEPNRSENTMDIFKCQQSMVSVLGWFANPIHGDGDYPEGMRKKLFSVLPIFSEAEKHEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGFSLKESTPDVQGQFPCDFSWGVTESVLKPESVASSPQFSDPHLYVWNATGNRLLHRVEGVRLKTRPAQCTDFVNIKKQLEMLARMKVTHYRFALDWASVLPTGNLSAVNRQALRYYRCVVSEGLKLGISAMVTLYYPTHAHLGLPEPLLHADGWLNPSTAEAFQAYAGLCFQELGDLVKLWITINEPNRLSDIYNRSGNDTYGAAHNLLVAHALAWRLYDRQFRPSQRGAVSLSLHADWAEPANPYADSHWRAAERFLQFEIAWFAEPLFKTGDYPAAMREYIASKHRRGLSSSALPRLTEAERRLLKGTVDFCALNHFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGFPFENSSSRCSQTQENTECTVCLFLVQKKPLIFLGCCFFSTLVLLLSIAIFQRQKRRKFWKAKNLQHIPLKKGKRVVS

SEQ ID NO: 3 - protein, 567 aa, Artificial Sequence
  synthetic sequence of sp_Q6UWM7_LCTL_HUMAN Lactase-like protein OS=Homo sapiens
MKPVWVATLLWMLLLVPRLGAARKGSPEEASFYYGTFPLGFSWGVGSSAYQTEGAWDQDGKGPSIWDVFTHSGKGKVLGNETADVACDGYYKVQEDIILLRELHVNHYRFSLSWPRLLPTGIRAEQVNKKGIEFYSDLIDALLSSNITPIVTLHHWDLPQLLQVKYGGWQNVSMANYFRDYANLCFEAFGDRVKHWITFSDPRAMAEKGYETGHHAPGLKLRGTGLYKAAHHIIKAHAKAWHSYNTTWRSKQQGLVGISLNCDWGEPVDISNPKDLEAAERYLQFCLGWFANPIYAGDYPQVMKDYIGRKSAEQGLEMSRLPVFSLQEKSYIKGTSDFLGLGHFTTRYITERNYPSRQGPSYQNDRDLIELVDPNWPDLGSKWLYSVPWGFRRLLNFAQTQYGDPPIYVMENGASQKFHCTQLCDEWRIQYLKGYINEMLKAIKDGANIKGYTSWSLLDKFEWEKGYSDRYGFYYVEFNDRNKPRYPKASVQYYKKIIIANGFPNPREVESWYLKALETCSINNQMLAAEPLLSHMQMVTEIVVPTVCSLCVLITAVLLMLLLRRQS

SEQ ID NO: 4 - protein, 549 aa, Artificial Sequence
  synthetic sequence of sp_Q9UEF7-2|KLOT_HUMAN Isoform 2 of Klotho OS=Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVSQLTKPISSLTKPYH

SEQ ID NO: 5 - protein, 394 aa, Artificial Sequence
  synthetic sequence of sp_Q6UWM7-2_LCTL_HUMAN Isoform 2 of Lactase-like protein OS=Homo sapiens
MANYFRDYANLCFEAFGDRVKHWITFSDPRAMAEKGYETGHHAPGLKLRGTGLYKAAHHIIKAHAKAWHSYNTTWRSKQQGLVGISLNCDWGEPVDISNPKDLEAAERYLQFCLGWFANPIYAGDYPQVMKDYIGRKSAEQGLEMSRLPVFSLQEKSYIKGTSDFLGLGHFTTRYITERNYPSRQGPSYQNDRDLIELVDPNWPDLGSKWLYSVPWGFRRLLNFAQTQYGDPPIYVMENGASQKFHCTQLCDEWRIQYLKGYINEMLKAIKDGANIKGYTSWSLLDKFEWEKGYSDRYGFYYVEFNDRNKPRYPKASVQYYKKIIIANGFPNPREVESWYLKALETCSINNQMLAAEPLLSHMQMVTEIVVPTVCSLCVLITAVLLMLLLRRQS